SEQUENCE LISTING (294 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 214 aa, Homo sapiens
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 3 - protein, 225 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRYSFQVPGPLVWTLSPLFPSLCAERM

SEQ ID NO: 4 - protein, 150 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFRCSTLSFSWLVDS

SEQ ID NO: 5 - protein, 233 aa, Macaca mulatta
MRKTRLWGLLWMLFVSELRATTELTEEKYEYKEGQTLEVKCDYALEKYANSRKAWQKMEGKMPKILAKTERPSENSHPVQVGRITLEDYPDHGLLQVQMTNLQVEDSGLYQCVIYQHPKESHVLFNPICLVVTKGSSGTPGSSENSTQNVYRTPSTTAKALGPRYTSPRTVTQAPPESTVVVSTPGSEINLTNVTDIIRVPVFNIVIIVAGGFLSKSLVFSVLFAVTLRSFGP

SEQ ID NO: 6 - protein, 196 aa, Homo sapiens
MSRRSMLLAWALPSLLRLGAAQETEDPACCSPIVPRNEWKALASECAQHLSLPLRYVVVSHTAGSSCNTPASCQQQARNVQHYHMKTLGWCDVGYNFLIGEDGLVYEGRGWNFTGAHSGHLWNPMSIGISFMGNYMDRVPTPQAIRAAQGLLACGVAQGALRSNYVLKGHRDVQRTLSPGNQLYHLIQNWPHYRSP

SEQ ID NO: 7 - protein, 263 aa, Artificial Sequence
  CID101904 hTREM1_1-200-Avi-Tev-HKH
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRLEGGGSGGSGGLNDIFEAQKIEWHENLYFQGGSHHHHHHKAKGSKGKGSKKAGHHHHHHHHHH

SEQ ID NO: 8 - protein, 264 aa, Artificial Sequence
  CID101953 cTREM1_1-201-Avi-Tev-HKH
MRKTRLWGLLWMLFVSELRATTELTEEKYEYKEGQTLEVKCDYALEKYANSRKAWQKMEGKMPKILAKTERPSENSHPVQVGRITLEDYPDHGLLQVRMTNLQVEDSGLYQCVIYQHPKESHVLFNPICLVVTKGSSGTPGSSENSTQNVYRTPSTTAKALGPRYTSPRTVTQAPPESTVVVSTPGSEINLTNVTDIIRVPLEGGGSGGSGGLNDIFEAQKIEWHENLYFQGGSHHHHHHKAKGSKGKGSKKAGHHHHHHHHHH

SEQ ID NO: 9 - protein, 215 aa, Artificial Sequence
  CID101907 hTREM1 IgV (His-Smt-TREM1_21-139)
MGHHHHHHSGEVKPEVKPETHINLKVSDGSSEIFFKIKKTTPLRRLMEAFAKRQGKEMDSLRFLYDGIRIQADQTPEDLDMEDNDIIEAHREQIGGATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSG

SEQ ID NO: 10 - protein, 202 aa, Artificial Sequence
  CID101951 hPGLYRP1_1-196-His
MSRRSMLLAWALPSLLRLGAAQETEDPACCSPIVPRNEWKALASECAQHLSLPLRYVVVSHTAGSSCNTPASCQQQARNVQHYHMKTLGWCDVGYNFLIGEDGLVYEGRGWNFTGAHSGHLWNPMSIGISFMGNYMDRVPTPQAIRAAQGLLACGVAQGALRSNYVLKGHRDVQRTLSPGNQLYHLIQNWPHYRSPHHHHHH

SEQ ID NO: 11 - protein, 11 aa, Artificial Sequence
  CDRL1
QASQNIGSDLA

SEQ ID NO: 12 - protein, 7 aa, Artificial Sequence
  CDRL2
KAATLAS

SEQ ID NO: 13 - protein, 14 aa, Artificial Sequence
  CDRL3
QQYYYGSAGADTDT

SEQ ID NO: 14 - protein, 10 aa, Artificial Sequence
  CDRH1
GFSLSSYAMT

SEQ ID NO: 15 - protein, 16 aa, Artificial Sequence
  CDRH2
IIYAGGSPSYASWAKG

SEQ ID NO: 16 - protein, 12 aa, Artificial Sequence
  CDRH3
GTGDTVYTYFNI

SEQ ID NO: 17 - protein, 112 aa, Artificial Sequence
  Rabbit Ab 12172 VL region
AVVLTQTASPVSAPVGGTVTIKCQASQNIGSDLAWYQQEPGQPPKLLIYKAATLASGVPSRFKGSGSGTEFTLTISGVQCEDGATYYCQQYYYGSAGADTDTFGGGTEVVVK

SEQ ID NO: 18 - DNA, 336 bp, Artificial Sequence
  Rabbit Ab 12172 VL region
gccgtcgtgctgacccagactgcatcccccgtgtctgcacctgtgggaggcacagtcaccatcaagtgccaggccagtcagaacattggtagcgacttagcctggtatcagcaggaaccagggcagccacccaagctcctgatctacaaggcagccactctggcatctggggtcccatcgcggttcaaaggcagtggatctgggacagagttcactctcaccatcagtggcgtgcagtgtgaagatggtgccacttactactgtcaacagtattattatggtagtgctggtgctgatacggatactttcggcggagggaccgaggtggtggtcaaa

SEQ ID NO: 19 - protein, 216 aa, Artificial Sequence
  CID102770 Rabbit Ab 12172 light chain (Fab)
AVVLTQTASPVSAPVGGTVTIKCQASQNIGSDLAWYQQEPGQPPKLLIYKAATLASGVPSRFKGSGSGTEFTLTISGVQCEDGATYYCQQYYYGSAGADTDTFGGGTEVVVKRTPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 20 - DNA, 648 bp, Artificial Sequence
  Rabbit Ab 12172 light chain
gccgtcgtgctgacccagactgcatcccccgtgtctgcacctgtgggaggcacagtcaccatcaagtgccaggccagtcagaacattggtagcgacttagcctggtatcagcaggaaccagggcagccacccaagctcctgatctacaaggcagccactctggcatctggggtcccatcgcggttcaaaggcagtggatctgggacagagttcactctcaccatcagtggcgtgcagtgtgaagatggtgccacttactactgtcaacagtattattatggtagtgctggtgctgatacggatactttcggcggagggaccgaggtggtggtcaaacgtacgccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 21 - protein, 117 aa, Artificial Sequence
  Rabbit Ab 12172 VH region
QSLEESGGRLVTPGTPLTLTCTASGFSLSSYAMTWVRQAPGKGLEWIGIIYAGGSPSYASWAKGRFTISKTSTTVDLKMTSPTTEDTATYFCARGTGDTVYTYFNIWGPGTLVTVSS

SEQ ID NO: 22 - DNA, 351 bp, Artificial Sequence
  Rabbit Ab 12172 VH region
cagtcgctggaggagtccgggggtcgcctggtcacgcctgggacacccctgacactcacctgcacagcctctggattctccctcagtagctatgcaatgacctgggtccgccaggctccagggaaggggctggaatggatcggaatcatttatgctggtggtagcccatcctacgcgagctgggcgaaaggccgattcaccatctccaaaacctcgaccacggtggatctgaaaatgaccagtccgacaaccgaggacacggccacctatttctgtgccagagggactggtgatactgtttatacctactttaatatttggggcccaggcaccctggtcaccgtctcgagt

SEQ ID NO: 23 - protein, 220 aa, Artificial Sequence
  CID120769 Rabbit Ab 12172 heavy chain (Fab)
QSLEESGGRLVTPGTPLTLTCTASGFSLSSYAMTWVRQAPGKGLEWIGIIYAGGSPSYASWAKGRFTISKTSTTVDLKMTSPTTEDTATYFCARGTGDTVYTYFNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKP

SEQ ID NO: 24 - DNA, 660 bp, Artificial Sequence
  Rabbit Ab 12172 heavy chain (Fab)
cagtcgctggaggagtccgggggtcgcctggtcacgcctgggacacccctgacactcacctgcacagcctctggattctccctcagtagctatgcaatgacctgggtccgccaggctccagggaaggggctggaatggatcggaatcatttatgctggtggtagcccatcctacgcgagctgggcgaaaggccgattcaccatctccaaaacctcgaccacggtggatctgaaaatgaccagtccgacaaccgaggacacggccacctatttctgtgccagagggactggtgatactgtttatacctactttaatatttggggcccaggcaccctggtcaccgtctcgagtgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagccc

SEQ ID NO: 25 - protein, 112 aa, Artificial Sequence
  12172gL1 V-region
AVVLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIK

SEQ ID NO: 26 - DNA, 336 bp, Artificial Sequence
  12172gL1 V-region
gccgtggtgctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaag

SEQ ID NO: 27 - protein, 219 aa, Artificial Sequence
  12172gL1 light chain
AVVLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 657 bp, Artificial Sequence
  12172gL1 light chain
gccgtggtgctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - protein, 112 aa, Artificial Sequence
  12172gL2 V-region
DIQLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIK

SEQ ID NO: 30 - DNA, 336 bp, Artificial Sequence
  12172gL2 V-region
gacatccagctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaag

SEQ ID NO: 31 - protein, 219 aa, Artificial Sequence
  12172gL2 light chain
DIQLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 657 bp, Artificial Sequence
  12172gL2 light chain
gacatccagctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 33 - protein, 112 aa, Artificial Sequence
  12172gL6 V-region
DIVLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIK

SEQ ID NO: 34 - DNA, 336 bp, Artificial Sequence
  12172gL6 V-region
gacatcgtgctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaag

SEQ ID NO: 35 - protein, 219 aa, Artificial Sequence
  12172gL6 light chain
DIVLTQSPSFLSASVGDRVTITCQASQNIGSDLAWYQQKPGKAPKLLIYKAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 657 bp, Artificial Sequence
  12172gL6 light chain
gacatcgtgctgactcagtccccctccttcctgagcgcctcagtgggagatcgcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 37 - protein, 112 aa, Artificial Sequence
  12172gL9 V-region
DIQLTQSPSFLSASVGDSVTITCQASQNIGSDLAWYQQKPGKAPKLLIYKAATLASGVPSRFSGSGSGTEFTLTISSLQPEDFATYYCQQYYYGSAGADTDTFGGGTKVEIK

SEQ ID NO: 38 - DNA, 336 bp, Artificial Sequence
  12172gL9 V-region
gacatccagctgactcagtccccctccttcctgagcgcctcagtgggagatagcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaag

SEQ ID NO: 39 - protein, 219 aa, Artificial Sequence
  12172gL9 light chain
DIQLTQSPSFLSASVGDSVTITCQASQNIGSDLAWYQQKPGKAPKLLIYKAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 657 bp, Artificial Sequence
  12172gL9 light chain
gacatccagctgactcagtccccctccttcctgagcgcctcagtgggagatagcgtgaccatcacctgtcaagccagccagaacattgggtccgacttggcctggtatcagcagaagcctggcaaagcgccgaagctgctgatctacaaggccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 41 - protein, 112 aa, Artificial Sequence
  12172gL11 V-region
DIQLTQSPSFLSASVGDRVTITCQASQNIGSDLAWYQQKPGKAPKLLIYSAATLASGVPSRFSGSGSGTEFTLTISSLQPEDFATYYCQQYYYGSAGADTDTFGGGTKVEIK

SEQ ID NO: 42 - DNA, 336 bp, Artificial Sequence
  12172gL11 V-region
gacatccagctgactcagtccccctccttcctgagcgcctcagtgggagatcgcgtgaccatcacctgtcaagccagccagaacattgggtccgacttggcctggtatcagcagaagcctggcaaagcgccgaagctgctgatctacagcgccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaag

SEQ ID NO: 43 - protein, 219 aa, Artificial Sequence
  12172gL11 light chain
DIQLTQSPSFLSASVGDRVTITCQASQNIGSDLAWYQQKPGKAPKLLIYSAATLASGVPSRFSGSGSGTEFTLTISSLQPEDFATYYCQQYYYGSAGADTDTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 657 bp, Artificial Sequence
  12172gL11 light chain
gacatccagctgactcagtccccctccttcctgagcgcctcagtgggagatcgcgtgaccatcacctgtcaagccagccagaacattgggtccgacttggcctggtatcagcagaagcctggcaaagcgccgaagctgctgatctacagcgccgctactctcgcgtcgggagtgccatcccggttttccggttcgggaagcgggaccgagttcacccttaccatttcctcgctccaaccggaggacttcgccacgtactactgccagcagtactactacggttcagccggagcagacactgataccttcggcggaggcaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 45 - protein, 120 aa, Artificial Sequence
  12172gH1 V-region
EVQLVESGGGLVQPGGSLRLSCTASGFSLSSYAMTWVRQAPGKGLEWIGIIYAGGSPSYASWAKGRFTISKDSSKNTVYLQMNSLRAEDTAVYYCARGTGDTVYTYFNIWGQGTLVTVSS

SEQ ID NO: 46 - DNA, 360 bp, Artificial Sequence
  12172gH1 V-region
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagc

SEQ ID NO: 47 - protein, 447 aa, Artificial Sequence
  12172gH1 heavy chain (IgG4P)
EVQLVESGGGLVQPGGSLRLSCTASGFSLSSYAMTWVRQAPGKGLEWIGIIYAGGSPSYASWAKGRFTISKDSSKNTVYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - DNA, 1341 bp, Artificial Sequence
  12172gH1 heavy chain (IgG4P)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 49 - protein, 120 aa, Artificial Sequence
  12172gH2 V-region
EVQLVESGGGLVQPGGSLRLSCAASGFSLSSYAMTWVRQAPGKGLEWVGIIYAGGSPSYASWAKGRFTISKDSSKNTVYLQMNSLRAEDTAVYYCARGTGDTVYTYFNIWGQGTLVTVSS

SEQ ID NO: 50 - DNA, 360 bp, Artificial Sequence
  12172gH2 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatgggtcggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagc

SEQ ID NO: 51 - protein, 447 aa, Artificial Sequence
  12172gH2 heavy chain (IgG4P)
EVQLVESGGGLVQPGGSLRLSCAASGFSLSSYAMTWVRQAPGKGLEWVGIIYAGGSPSYASWAKGRFTISKDSSKNTVYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - DNA, 1341 bp, Artificial Sequence
  12172gH2 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatgggtcggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 53 - protein, 120 aa, Artificial Sequence
  12172gH4 V-region
EVQLVESGGGLVQPGGSLRLSCTASGFSLSSYAMTWVRQAPGKGLEWIGIIYAGGSPSYASWAKGRFTISRDSSKNTVYLQMNSLRAEDTAVYYCARGTGDTVYTYFNIWGQGTLVTVSS

SEQ ID NO: 54 - DNA, 360 bp, Artificial Sequence
  12172gH4 V-region
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgactcctccaagaacacggtgtacttgcaaatgaactcactgagggccgaggacactgcggtgtactactgtgcccggggaactggagataccgtgtacacctactttaacatctggggccagggaaccctggtcactgtctcgagc

SEQ ID NO: 55 - protein, 447 aa, Artificial Sequence
  12172gH4 heavy chain (IgG4P)
EVQLVESGGGLVQPGGSLRLSCTASGFSLSSYAMTWVRQAPGKGLEWIGIIYAGGSPSYASWAKGRFTISRDSSKNTVYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - DNA, 1341 bp, Artificial Sequence
  12172gH4 heavy chain (IgG4P)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgactcctccaagaacacggtgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 57 - protein, 120 aa, Artificial Sequence
  12172gH6 V-region
EVQLVESGGGLVQPGGSLRLSCTASGFSLSSYAMTWVRQAPGKGLEWIGIIYAGGSPSYASWAKGRFTISKDSSKNTLYLQMNSLRAEDTAVYYCARGTGDTVYTYFNIWGQGTLVTVSS

SEQ ID NO: 58 - DNA, 360 bp, Artificial Sequence
  12172gH6 V-region
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 59 - protein, 447 aa, Artificial Sequence
  12172gH6 heavy chain (IgG4P)
EVQLVESGGGLVQPGGSLRLSCTASGFSLSSYAMTWVRQAPGKGLEWIGIIYAGGSPSYASWAKGRFTISKDSSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - DNA, 1341 bp, Artificial Sequence
  12172gH6 heavy chain (IgG4P)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 61 - protein, 447 aa, Artificial Sequence
  12172gH6 heavy chain (IgG4P F234A L235A)
EVQLVESGGGLVQPGGSLRLSCTASGFSLSSYAMTWVRQAPGKGLEWIGIIYAGGSPSYASWAKGRFTISKDSSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - DNA, 1341 bp, Artificial Sequence
  12172gH6 heavy chain (IgG4P F234A L235A)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaagccgcggggggaccgtcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 63 - protein, 450 aa, Artificial Sequence
  12172gH6 heavy chain (IgG1)
EVQLVESGGGLVQPGGSLRLSCTASGFSLSSYAMTWVRQAPGKGLEWIGIIYAGGSPSYASWAKGRFTISKDSSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1350 bp, Artificial Sequence
  12172gH6 heavy chain (IgG1)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 65 - protein, 450 aa, Artificial Sequence
  12172gH6 heavy chain (IgG1 L234A L235A)
EVQLVESGGGLVQPGGSLRLSCTASGFSLSSYAMTWVRQAPGKGLEWIGIIYAGGSPSYASWAKGRFTISKDSSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1350 bp, Artificial Sequence
  12172gH6 heavy chain (IgG1 L234A L235A)
gaagtgcagctcgtggagtcgggcggaggtctggtgcagcccggaggaagcctgcgcctgtcatgcaccgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaagccgcaggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggatgaactgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 67 - protein, 120 aa, Artificial Sequence
  12172gH8 V-region
EVQLVESGGGLVQPGGSLRLSCAASGFSLSSYAMTWVRQAPGKGLEWIGIIYAGGSPSYASWAKGRFTISKDSSKNTVYLQMNSLRAEDTAVYYCARGTGDTVYTYFNIWGQGTLVTVSS

SEQ ID NO: 68 - DNA, 360 bp, Artificial Sequence
  12172gH8 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagc

SEQ ID NO: 69 - protein, 447 aa, Artificial Sequence
  12172gH8 heavy chain (IgG4P)
EVQLVESGGGLVQPGGSLRLSCAASGFSLSSYAMTWVRQAPGKGLEWIGIIYAGGSPSYASWAKGRFTISKDSSKNTVYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - DNA, 1341 bp, Artificial Sequence
  12172gH8 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggactcctccaagaacacggtgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 71 - protein, 120 aa, Artificial Sequence
  12172gH9 V-region
EVQLVESGGGLVQPGGSLRLSCAASGFSLSSYAMTWVRQAPGKGLEWIGIIYAGGSPSYASWAKGRFTISRDNSKNTVYLQMNSLRAEDTAVYYCARGTGDTVYTYFNIWGQGTLVTVSS

SEQ ID NO: 72 - DNA, 360 bp, Artificial Sequence
  12172gH9 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgacaattccaagaacacggtgtacttgcaaatgaactcactgagggccgaggacactgcggtgtactactgtgcccggggaactggagataccgtgtacacctactttaacatctggggccagggaaccctggtcactgtctcgagc

SEQ ID NO: 73 - protein, 447 aa, Artificial Sequence
  12172gH9 heavy chain (IgG4P)
EVQLVESGGGLVQPGGSLRLSCAASGFSLSSYAMTWVRQAPGKGLEWIGIIYAGGSPSYASWAKGRFTISRDNSKNTVYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - DNA, 1341 bp, Artificial Sequence
  12172gH9 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgacaattccaagaacacggtgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 75 - protein, 120 aa, Artificial Sequence
  12172gH10 V-region
EVQLVESGGGLVQPGGSLRLSCAASGFSLSSYAMTWVRQAPGKGLEWIGIIYAGGSPSYASWAKGRFTISRDSSKNTLYLQMNSLRAEDTAVYYCARGTGDTVYTYFNIWGQGTLVTVSS

SEQ ID NO: 76 - DNA, 360 bp, Artificial Sequence
  12172gH10 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgactcctccaagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 77 - protein, 447 aa, Artificial Sequence
  12172gH10 heavy chain (IgG4P)
EVQLVESGGGLVQPGGSLRLSCAASGFSLSSYAMTWVRQAPGKGLEWIGIIYAGGSPSYASWAKGRFTISRDSSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - DNA, 1341 bp, Artificial Sequence
  12172gH10 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgactcctccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 79 - protein, 120 aa, Artificial Sequence
  12172gH11 V-region
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSS

SEQ ID NO: 80 - DNA, 360 bp, Artificial Sequence
  12172gH11 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccaagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 81 - protein, 447 aa, Artificial Sequence
  12172gH11 heavy chain (IgG4P)
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - DNA, 1341 bp, Artificial Sequence
  12172gH11 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccctccgtgttccctctggccccttgctcccggtccacctccgagtctaccgccgctctgggctgcctggtcaaggactacttccccgagcccgtgacagtgtcctggaactctggcgccctgacctccggcgtgcacaccttccctgccgtgctgcagtcctccggcctgtactccctgtcctccgtcgtgaccgtgccctcctccagcctgggcaccaagacctacacctgtaacgtggaccacaagccctccaacaccaaggtggacaagcgggtggaatctaagtacggccctccctgccccccctgccctgcccctgaatttctgggcggaccttccgtgttcctgttccccccaaagcccaaggacaccctgatgatctcccggacccccgaagtgacctgcgtggtggtggacgtgtcccaggaagatcccgaggtccagttcaattggtacgtggacggcgtggaagtgcacaatgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtcaagggcttctacccctccgacattgccgtggaatgggagtccaacggccagcccgagaacaactacaagaccaccccccctgtgctggacagcgacggctccttcttcctgtactctcggctgaccgtggacaagtcccggtggcaggaaggcaacgtcttctcctgctccgtgatgcacgaggccctgcacaaccactacacccagaagtccctgtccctgagcctgggcaag

SEQ ID NO: 83 - protein, 447 aa, Artificial Sequence
  12172gH11 heavy chain (IgG4P F234A L235A)
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - DNA, 1341 bp, Artificial Sequence
  12172gH11 heavy chain (IgG4P F234A L235A)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaagccgcggggggaccgtcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 85 - protein, 450 aa, Artificial Sequence
  12172gH11 heavy chain (IgG1)
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1350 bp, Artificial Sequence
  12172gH11 heavy chain (IgG1)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - protein, 450 aa, Artificial Sequence
  12172gH11 heavy chain (IgG1 L234A L235A)
EVQLVESGGGLVQPGGSLRLSCAASGFSLSSYAMTWVRQAPGKGLEWIGIIYAGGSPSYASWAKGRFTISKDNSKNTLYLQMNSLRAEDTAVYYCARGTGDTVYTYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1350 bp, Artificial Sequence
  12172gH11 heavy chain (IgG1 L234A L235A)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaagccgcaggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggatgaactgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 89 - protein, 120 aa, Artificial Sequence
  12172gH12 V-region
EVQLVESGGGLVQPGGSLRLSCAASGFSLSSYAMTWVRQAPGKGLEWIGIIYAGGSPSYASWAKGRFTISRDNSKNTLYLQMNSLRAEDTAVYYCARGTGDTVYTYFNIWGQGTLVTVSS

SEQ ID NO: 90 - DNA, 360 bp, Artificial Sequence
  12172gH12 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgacaattccaagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 91 - protein, 447 aa, Artificial Sequence
  12172gH12 heavy chain (IgG4P)
EVQLVESGGGLVQPGGSLRLSCAASGFSLSSYAMTWVRQAPGKGLEWIGIIYAGGSPSYASWAKGRFTISRDNSKNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - DNA, 1341 bp, Artificial Sequence
  12172gH12 heavy chain (IgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgcgcgacaattccaagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 93 - protein, 120 aa, Artificial Sequence
  12172gH26 V-region
EVQLVESGGGLVQPGGSLRLSCAASGFSLSSYAMTWVRQAPGKGLEWIGIIYAGGSPSYASWAKGRFTISKDNSSNTLYLQMNSLRAEDTAVYYCARGTGDTVYTYFNIWGQGTLVTVSS

SEQ ID NO: 94 - DNA, 360 bp, Artificial Sequence
  12172gH26 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccagcaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 95 - protein, 447 aa, Artificial Sequence
  12172gH26 heavy chain (hIgG4P)
EVQLVESGGGLVQPGGSLRLSCAASGFSLSSYAMTWVRQAPGKGLEWIGIIYAGGSPSYASWAKGRFTISKDNSSNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - DNA, 1341 bp, Artificial Sequence
  12172gH26 heavy chain (hIgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccagcaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 97 - protein, 120 aa, Artificial Sequence
  12172gH48 V-region
EVQLVESGGGLVQPGGSLRLSCAASGFSLSSYAMTWVRQAPGKGLEWIGIIYAGGSPSYASWAKGRFTISKDNSQNTLYLQMNSLRAEDTAVYYCARGTGDTVYTYFNIWGQGTLVTVSS

SEQ ID NO: 98 - DNA, 360 bp, Artificial Sequence
  12172gH48 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattcccagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 99 - protein, 447 aa, Artificial Sequence
  12172gH48 heavy chain (hIgG4P)
EVQLVESGGGLVQPGGSLRLSCAASGFSLSSYAMTWVRQAPGKGLEWIGIIYAGGSPSYASWAKGRFTISKDNSQNTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - DNA, 1341 bp, Artificial Sequence
  12172gH48 heavy chain (hIgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattcccagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 101 - protein, 120 aa, Artificial Sequence
  12172gH49 V-region
EVQLVESGGGLVQPGGSLRLSCAASGFSLSSYAMTWVRQAPGKGLEWIGIIYAGGSPSYASWAKGRFTISKDNSENTLYLQMNSLRAEDTAVYYCARGTGDTVYTYFNIWGQGTLVTVSS

SEQ ID NO: 102 - DNA, 360 bp, Artificial Sequence
  12172gH49 V-region
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccgagaacacgctgtacttgcaaatgaactcactgagggccgaggacactgcggtgtactactgtgcccggggaactggagataccgtgtacacctactttaacatctggggccagggaaccctggtcactgtctcgagc

SEQ ID NO: 103 - protein, 447 aa, Artificial Sequence
  12172gH49 heavy chain (hIgG4P)
EVQLVESGGGLVQPGGSLRLSCAASGFSLSSYAMTWVRQAPGKGLEWIGIIYAGGSPSYASWAKGRFTISKDNSENTLYLQMNSLRAEDTAVYYCARGTGDTVYTYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - DNA, 1341 bp, Artificial Sequence
  12172gH49 heavy chain (hIgG4P)
gaagtgcagctcgtggagtcgggcggaggtctggtgcagcccggaggaagcctgcgcctgtcatgcgctgccagcgggttctcgctttcctcctacgccatgacctgggtcagacaggcaccgggaaagggcctcgaatggattggcattatctatgcgggtggcagcccttcctacgcctcctgggctaaagggcggttcaccatctcgaaggacaattccgagaacacgctgtacttgcaaatgaactcactgagggccgaggacactgcggtgtactactgtgcccggggaactggagataccgtgtacacctactttaacatctggggccagggaaccctggtcactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 105 - protein, 107 aa, Artificial Sequence
  Human IGKV1-9 IGKJ4 acceptor framework
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 106 - DNA, 321 bp, Artificial Sequence
  Human IGKV1-9 IGKJ4 acceptor framework
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggagatcaaa

SEQ ID NO: 107 - protein, 112 aa, Artificial Sequence
  Human IGHV3-66 IGHJ4 acceptor framework
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARYFDYWGQGTLVTVSS

SEQ ID NO: 108 - DNA, 336 bp, Artificial Sequence
  Human IGHV3-66 IGHJ4 acceptor framework
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagatactttgactactggggccaaggaaccctggtcaccgtctcctca

SEQ ID NO: 109 - protein, 121 aa, Artificial Sequence
  Human TREM1  WT 01_WT_3A
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 110 - protein, 121 aa, Artificial Sequence
  Human TREM1 mutant  02_6
MELRAAAKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 111 - protein, 121 aa, Artificial Sequence
  Human TREM1 mutant  03_6_7
MELRAAAALTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 112 - protein, 121 aa, Artificial Sequence
  Human TREM1 mutant  04_6_7_9
MELRAAAALAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 113 - protein, 121 aa, Artificial Sequence
  Human TREM1 mutant  05_7_8_9
MELRAATAAAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 114 - protein, 121 aa, Artificial Sequence
  Human TREM1 mutant  06_9_10_111
MELRAATKLAAEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVTLE

SEQ ID NO: 115 - protein, 121 aa, Artificial Sequence
  Human TREM1 mutant  07_9_10_11
MELRAATKLAAAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 116 - protein, 121 aa, Artificial Sequence
  Human TREM1 mutant  08_10_11_12
MELRAATKLTAAAYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 117 - protein, 121 aa, Artificial Sequence
  Human TREM1 mutant  09_11_12_13
MELRAATKLTEAAAELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 118 - protein, 121 aa, Artificial Sequence
  Human TREM1 mutant  10_16_118_119
MELRAATKLTEEKYELAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVAAE

SEQ ID NO: 119 - protein, 121 aa, Artificial Sequence
  Human TREM1 mutant  11_19_20_85
MELRAATKLTEEKYELKEGAALDVKCDYTLEKFASSQKAWQIIRDGEMPKTLACTERPSKNSHPVQVGRIILEDYHDHGLLRVRMANLQVEDSGLYQCVIYQPPKEPHMLFDRIRLVVTLE

SEQ ID NO: 120 - protein, 121 aa, Artificial Sequence
  Human TREM1 mutant  12_22_24_26
MELRAATKLTEEKYELKEGQTLAVACAYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 121 - protein, 121 aa, Artificial Sequence
  Human TREM1 mutant  13_24_26_28
MELRAATKLTEEKYELKEGQTLDVACAYALEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 122 - protein, 121 aa, Artificial Sequence
  Human TREM1 mutant  14_26_28_29
MELRAATKLTEEKYELKEGQTLDVKCAYAAEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 123 - protein, 121 aa, Artificial Sequence
  Human TREM1 mutant  15_28_29_30
MELRAATKLTEEKYELKEGQTLDVKCDYAAAKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 124 - protein, 121 aa, Artificial Sequence
  Human TREM1 mutant  16_29_30_31
MELRAATKLTEEKYELKEGQTLDVKCDYTAAAFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 125 - protein, 121 aa, Artificial Sequence
  Human TREM1 mutant  17_31_32_35
MELRAATKLTEEKYELKEGQTLDVKCDYTLEAAASAQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 126 - protein, 121 aa, Artificial Sequence
  Human TREM1 mutant  18_30_34
MELRAATKLTEEKYELKEGQTLDVKCDYTLAKFAASQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 127 - protein, 121 aa, Artificial Sequence
  Human TREM1 mutant  19_34_35_57
MELRAATKLTEEKYELKEGQTLDVKCDYTLEKFAAAQKAWQIIRDGEMPKTLACTERASKNSHPVQVGRIILEDYHDHGLLRVRMVNLQVEDSGLYQCVIYQPPKEPHMLFDRIRLVVTLE

SEQ ID NO: 128 - protein, 121 aa, Artificial Sequence
  Human TREM1 mutant  20_35_36_56
MELRAATKLTEEKYELKEGQTLDVKCDYTLEKFASAAKAWQIIRDGEMPKTLACTEAPSKNSHPVQVGRIILEDYHDHGLLRVRMVNLQVEDSGLYQCVIYQPPKEPHMLFDRIRLVVTLE

SEQ ID NO: 129 - protein, 121 aa, Artificial Sequence
  Human TREM1 mutant  21_40_49_50
MELRAATKLTEEKYELKEGQTLDVKCDYTLEKFASSQKAWAIIRDGEMPAALACTERPSKNSHPVQVGRIILEDYHDHGLLRVRMVNLQVEDSGLYQCVIYQPPKEPHMLFDRIRLVVTLE

SEQ ID NO: 130 - protein, 121 aa, Artificial Sequence
  Human TREM1 mutant  22_43_44_47
MELRAATKLTEEKYELKEGQTLDVKCDYTLEKFASSQKAWQIIAAGEAPKTLACTERPSKNSHPVQVGRIILEDYHDHGLLRVRMVNLQVEDSGLYQCVIYQPPKEPHMLFDRIRLVVTLE

SEQ ID NO: 131 - protein, 121 aa, Artificial Sequence
  Human TREM1 mutant  23_43_44_45
MELRAATKLTEEKYELKEGQTLDVKCDYTLEKFASSQKAWQIIAAAEMPKTLACTERPSKNSHPVQVGRIILEDYHDHGLLRVRMVNLQVEDSGLYQCVIYQPPKEPHMLFDRIRLVVTLE

SEQ ID NO: 132 - protein, 121 aa, Artificial Sequence
  Human TREM1 mutant  24_44_45_46
MELRAATKLTEEKYELKEGQTLDVKCDYTLEKFASSQKAWQIIRAAAMPKTLACTERPSKNSHPVQVGRIILEDYHDHGLLRVRMVNLQVEDSGLYQCVIYQPPKEPHMLFDRIRLVVTLE

SEQ ID NO: 133 - protein, 121 aa, Artificial Sequence
  Human TREM1 mutant  25_45_46_47
MELRAATKLTEEKYELKEGQTLDVKCDYTLEKFASSQKAWQIIRDAAAPKTLACTERPSKNSHPVQVGRIILEDYHDHGLLRVRMVNLQVEDSGLYQCVIYQPPKEPHMLFDRIRLVVTLE

SEQ ID NO: 134 - protein, 121 aa, Artificial Sequence
  Human TREM1 mutant  26_43_46_47
MELRAATKLTEEKYELKEGQTLDVKCDYTLEKFASSQKAWQIIADGAAPKTLACTERPSKNSHPVQVGRIILEDYHDHGLLRVRMVNLQVEDSGLYQCVIYQPPKEPHMLFDRIRLVVTLE

SEQ ID NO: 135 - protein, 121 aa, Artificial Sequence
  Human TREM1 mutant  27_47_48_49
MELRAATKLTEEKYELKEGQTLDVKCDYTLEKFASSQKAWQIIRDGEAAATLACTERPSKNSHPVQVGRIILEDYHDHGLLRVRMVNLQVEDSGLYQCVIYQPPKEPHMLFDRIRLVVTLE

SEQ ID NO: 136 - protein, 121 aa, Artificial Sequence
  Human TREM1 mutant  28_47_49_50
MELRAATKLTEEKYELKEGQTLDVKCDYTLEKFASSQKAWQIIRDGEAPAALACTERPSKNSHPVQVGRIILEDYHDHGLLRVRMVNLQVEDSGLYQCVIYQPPKEPHMLFDRIRLVVTLE

SEQ ID NO: 137 - protein, 121 aa, Artificial Sequence
  Human TREM1 mutant  29_49_50_53
MELRAATKLTEEKYELKEGQTLDVKCDYTLEKFASSQKAWQIIRDGEMPAALAATERPSKNSHPVQVGRIILEDYHDHGLLRVRMVNLQVEDSGLYQCVIYQPPKEPHMLFDRIRLVVTLE

SEQ ID NO: 138 - protein, 121 aa, Artificial Sequence
  Human TREM1 mutant  30_50_53_55
MELRAATKLTEEKYELKEGQTLDVKCDYTLEKFASSQKAWQIIRDGEMPKALAATARPSKNSHPVQVGRIILEDYHDHGLLRVRMVNLQVEDSGLYQCVIYQPPKEPHMLFDRIRLVVTLE

SEQ ID NO: 139 - protein, 121 aa, Artificial Sequence
  Human TREM1 mutant  31_55_56_57
MELRAATKLTEEKYELKEGQTLDVKCDYTLEKFASSQKAWQIIRDGEMPKTLACTAAASKNSHPVQVGRIILEDYHDHGLLRVRMVNLQVEDSGLYQCVIYQPPKEPHMLFDRIRLVVTLE

SEQ ID NO: 140 - protein, 121 aa, Artificial Sequence
  Human TREM1 mutant  32_34_56_57
MELRAATKLTEEKYELKEGQTLDVKCDYTLEKFAASQKAWQIIRDGEMPKTLACTEAASKNSHPVQVGRIILEDYHDHGLLRVRMVNLQVEDSGLYQCVIYQPPKEPHMLFDRIRLVVTLE

SEQ ID NO: 141 - protein, 121 aa, Artificial Sequence
  Human TREM1 mutant  33_57_59_60
MELRAATKLTEEKYELKEGQTLDVKCDYTLEKFASSQKAWQIIRDGEMPKTLACTERASAASHPVQVGRIILEDYHDHGLLRVRMVNLQVEDSGLYQCVIYQPPKEPHMLFDRIRLVVTLE

SEQ ID NO: 142 - protein, 121 aa, Artificial Sequence
  Human TREM1 mutant  34_59_60_61
MELRAATKLTEEKYELKEGQTLDVKCDYTLEKFASSQKAWQIIRDGEMPKTLACTERPSAAAHPVQVGRIILEDYHDHGLLRVRMVNLQVEDSGLYQCVIYQPPKEPHMLFDRIRLVVTLE

SEQ ID NO: 143 - protein, 121 aa, Artificial Sequence
  Human TREM1 mutant  35_60_61_62
MELRAATKLTEEKYELKEGQTLDVKCDYTLEKFASSQKAWQIIRDGEMPKTLACTERPSKAAAPVQVGRIILEDYHDHGLLRVRMVNLQVEDSGLYQCVIYQPPKEPHMLFDRIRLVVTLE

SEQ ID NO: 144 - protein, 121 aa, Artificial Sequence
  Human TREM1 mutant  36_61_62_63
MELRAATKLTEEKYELKEGQTLDVKCDYTLEKFASSQKAWQIIRDGEMPKTLACTERPSKNAAAVQVGRIILEDYHDHGLLRVRMVNLQVEDSGLYQCVIYQPPKEPHMLFDRIRLVVTLE

SEQ ID NO: 145 - protein, 121 aa, Artificial Sequence
  Human TREM1 mutant  37_62_63_64
MELRAATKLTEEKYELKEGQTLDVKCDYTLEKFASSQKAWQIIRDGEMPKTLACTERPSKNSAAAQVGRIILEDYHDHGLLRVRMVNLQVEDSGLYQCVIYQPPKEPHMLFDRIRLVVTLE

SEQ ID NO: 146 - protein, 121 aa, Artificial Sequence
  Human TREM1 mutant  38_63_64_65
MELRAATKLTEEKYELKEGQTLDVKCDYTLEKFASSQKAWQIIRDGEMPKTLACTERPSKNSHAAAVGRIILEDYHDHGLLRVRMVNLQVEDSGLYQCVIYQPPKEPHMLFDRIRLVVTLE

SEQ ID NO: 147 - protein, 121 aa, Artificial Sequence
  Human TREM1 mutant  39_64_65_66
MELRAATKLTEEKYELKEGQTLDVKCDYTLEKFASSQKAWQIIRDGEMPKTLACTERPSKNSHPAAAGRIILEDYHDHGLLRVRMVNLQVEDSGLYQCVIYQPPKEPHMLFDRIRLVVTLE

SEQ ID NO: 148 - protein, 121 aa, Artificial Sequence
  Human TREM1 mutant  40_60_74_76
MELRAATKLTEEKYELKEGQTLDVKCDYTLEKFASSQKAWQIIRDGEMPKTLACTERPSKASHPVQVGRIILEDAHAHGLLRVRMVNLQVEDSGLYQCVIYQPPKEPHMLFDRIRLVVTLE

SEQ ID NO: 149 - protein, 121 aa, Artificial Sequence
  Human TREM1 mutant  41_60_75_76
MELRAATKLTEEKYELKEGQTLDVKCDYTLEKFASSQKAWQIIRDGEMPKTLACTERPSKASHPVQVGRIILEDYAAHGLLRVRMVNLQVEDSGLYQCVIYQPPKEPHMLFDRIRLVVTLE

SEQ ID NO: 150 - protein, 121 aa, Artificial Sequence
  Human TREM1 mutant  42_60_76_77
MELRAATKLTEEKYELKEGQTLDVKCDYTLEKFASSQKAWQIIRDGEMPKTLACTERPSKASHPVQVGRIILEDYHAAGLLRVRMVNLQVEDSGLYQCVIYQPPKEPHMLFDRIRLVVTLE

SEQ ID NO: 151 - protein, 121 aa, Artificial Sequence
  Human TREM1 mutant  43_28_76_77
MELRAATKLTEEKYELKEGQTLDVKCDYALEKFASSQKAWQIIRDGEMPKTLACTERPSKNSHPVQVGRIILEDYHAAGLLRVRMVNLQVEDSGLYQCVIYQPPKEPHMLFDRIRLVVTLE

SEQ ID NO: 152 - protein, 121 aa, Artificial Sequence
  Human TREM1 mutant  44_28_77_78
MELRAATKLTEEKYELKEGQTLDVKCDYALEKFASSQKAWQIIRDGEMPKTLACTERPSKNSHPVQVGRIILEDYHDAALLRVRMVNLQVEDSGLYQCVIYQPPKEPHMLFDRIRLVVTLE

SEQ ID NO: 153 - protein, 121 aa, Artificial Sequence
  Human TREM1 mutant  45_96_109_111
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYACVIYQPPKEPHMAFARIRLVVTLE

SEQ ID NO: 154 - protein, 121 aa, Artificial Sequence
  Human TREM1 mutant  46_98_100_109
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCAIAQPPKEPHMAFDRIRLVVTLE

SEQ ID NO: 155 - protein, 121 aa, Artificial Sequence
  Human TREM1 mutant  47_35_100_102
MELRAATKLTEEKYELKEGQTLDVKCDYTLEKFASAQKAWQIIRDGEMPKTLACTERPSKNSHPVQVGRIILEDYHDHGLLRVRMVNLQVEDSGLYQCVIAQAPKEPHMLFDRIRLVVTLE

SEQ ID NO: 156 - protein, 121 aa, Artificial Sequence
  Human TREM1 mutant  48_102_103_104
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQAAAEPHMLFDRIRLVVTLE

SEQ ID NO: 157 - protein, 121 aa, Artificial Sequence
  Human TREM1 mutant  49_103_104_105
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPAAAPHMLFDRIRLVVTLE

SEQ ID NO: 158 - protein, 121 aa, Artificial Sequence
  Human TREM1 mutant  50_104_105_106
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPAAAHMLFDRIRLVVTLE

SEQ ID NO: 159 - protein, 121 aa, Artificial Sequence
  Human TREM1 mutant  51_105_106_107
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKAAAMLFDRIRLVVTLE

SEQ ID NO: 160 - protein, 121 aa, Artificial Sequence
  Human TREM1 mutant  52_106_107_108
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEAAALFDRIRLVVTLE

SEQ ID NO: 161 - protein, 121 aa, Artificial Sequence
  Human TREM1 mutant  53_107_108_109
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPAAAFDRIRLVVTLE

SEQ ID NO: 162 - protein, 121 aa, Artificial Sequence
  Human TREM1 mutant  54_109_110_111
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMAAARIRLVVTLE

SEQ ID NO: 163 - protein, 121 aa, Artificial Sequence
  Human TREM1 mutant  55_11_113_114
MELRAATKLTEAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRAALVVTLE

SEQ ID NO: 164 - protein, 121 aa, Artificial Sequence
  Human TREM1 mutant  56_10_12_114
MELRAATKLTAEAYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIALVVTLE

SEQ ID NO: 165 - protein, 121 aa, Artificial Sequence
  Human TREM1 mutant  57_118_119_120
MELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVAAA

SEQ ID NO: 166 - protein, 121 aa, Artificial Sequence
  Human TREM1 mutant  58_16_119_120
MELRAATKLTEEKYELAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTAA

SEQ ID NO: 167 - protein, 117 aa, Artificial Sequence
  Human TREM1 WT 01_WT_2A
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 168 - protein, 117 aa, Artificial Sequence
  Human TREM1 mutant  02_2_3
AAAALTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 169 - protein, 117 aa, Artificial Sequence
  Human TREM1 mutant  03_3_4
AATAATEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 170 - protein, 117 aa, Artificial Sequence
  Human TREM1 mutant  04_4_5
AATKAAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 171 - protein, 117 aa, Artificial Sequence
  Human TREM1 mutant  05_5_104
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALFDRIRLVVTKG

SEQ ID NO: 172 - protein, 117 aa, Artificial Sequence
  Human TREM1 mutant  06_5_106
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLADRIRLVVTKG

SEQ ID NO: 173 - protein, 117 aa, Artificial Sequence
  Human TREM1 mutant  07_5_107
AATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVTKG

SEQ ID NO: 174 - protein, 117 aa, Artificial Sequence
  Human TREM1 mutant  08_7_9
AATKLTEAKAELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 175 - protein, 117 aa, Artificial Sequence
  Human TREM1 mutant  09_7_107
AATKLTEAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVTKG

SEQ ID NO: 176 - protein, 117 aa, Artificial Sequence
  Human TREM1 mutant  10_8_10
AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 177 - protein, 117 aa, Artificial Sequence
  Human TREM1 mutant  11_11_12
AATKLTEEKYEAAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 178 - protein, 117 aa, Artificial Sequence
  Human TREM1 mutant  12_12_13
AATKLTEEKYELAAGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 179 - protein, 117 aa, Artificial Sequence
  Human TREM1 mutant  13_12_15
AATKLTEEKYELAEGATLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 180 - protein, 117 aa, Artificial Sequence
  Human TREM1 mutant  14_13_85
AATKLTEEKYELKAGQTLDVKCDYTLEKFASSQKAWQIIRDGEMPKTLACTERPSKNSHPVQVGRIILEDYHDHGLLRVRMVNLQAEDSGLYQCVIYQPPKEPHMLFDRIRLVVTKG

SEQ ID NO: 181 - protein, 117 aa, Artificial Sequence
  Human TREM1 mutant  15_16_81
AATKLTEEKYELKEGQALDVKCDYTLEKFASSQKAWQIIRDGEMPKTLACTERPSKNSHPVQVGRIILEDYHDHGLLRVRMANLQVEDSGLYQCVIYQPPKEPHMLFDRIRLVVTKG

SEQ ID NO: 182 - protein, 117 aa, Artificial Sequence
  Human TREM1 mutant  16_20_75
AATKLTEEKYELKEGQTLDVACDYTLEKFASSQKAWQIIRDGEMPKTLACTERPSKNSHPVQVGRIILEDYHDHGALRVRMVNLQVEDSGLYQCVIYQPPKEPHMLFDRIRLVVTKG

SEQ ID NO: 183 - protein, 117 aa, Artificial Sequence
  Human TREM1 mutant  17_24_25
AATKLTEEKYELKEGQTLDVKCDYAAEKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 184 - protein, 117 aa, Artificial Sequence
  Human TREM1 mutant  18_24_26
AATKLTEEKYELKEGQTLDVKCDYALAKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 185 - protein, 117 aa, Artificial Sequence
  Human TREM1 mutant  19_24_27
AATKLTEEKYELKEGQTLDVKCDYALEAFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 186 - protein, 117 aa, Artificial Sequence
  Human TREM1 mutant  20_25_26
AATKLTEEKYELKEGQTLDVKCDYTAAKFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 187 - protein, 117 aa, Artificial Sequence
  Human TREM1 mutant  21_26_27
AATKLTEEKYELKEGQTLDVKCDYTLAAFASSQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 188 - protein, 117 aa, Artificial Sequence
  Human TREM1 mutant  22_30_31
AATKLTEEKYELKEGQTLDVKCDYTLEKFAAAQKAWQIIRDGEMPKTLACTERPSKNSHPVQVGRIILEDYHDHGLLRVRMVNLQVEDSGLYQCVIYQPPKEPHMLFDRIRLVVTKG

SEQ ID NO: 189 - protein, 117 aa, Artificial Sequence
  Human TREM1 mutant  23_30_53
AATKLTEEKYELKEGQTLDVKCDYTLEKFAASQKAWQIIRDGEMPKTLACTERASKNSHPVQVGRIILEDYHDHGLLRVRMVNLQVEDSGLYQCVIYQPPKEPHMLFDRIRLVVTKG

SEQ ID NO: 190 - protein, 117 aa, Artificial Sequence
  Human TREM1 mutant  24_37_38
AATKLTEEKYELKEGQTLDVKCDYTLEKFASSQKAWQAARDGEMPKTLACTERPSKNSHPVQVGRIILEDYHDHGLLRVRMVNLQVEDSGLYQCVIYQPPKEPHMLFDRIRLVVTKG

SEQ ID NO: 191 - protein, 117 aa, Artificial Sequence
  Human TREM1 mutant  25_37_39
AATKLTEEKYELKEGQTLDVKCDYTLEKFASSQKAWQAIADGEMPKTLACTERPSKNSHPVQVGRIILEDYHDHGLLRVRMVNLQVEDSGLYQCVIYQPPKEPHMLFDRIRLVVTKG

SEQ ID NO: 192 - protein, 117 aa, Artificial Sequence
  Human TREM1 mutant  26_37_45
AATKLTEEKYELKEGQTLDVKCDYTLEKFASSQKAWQAIRDGEMPATLACTERPSKNSHPVQVGRIILEDYHDHGLLRVRMVNLQVEDSGLYQCVIYQPPKEPHMLFDRIRLVVTKG

SEQ ID NO: 193 - protein, 117 aa, Artificial Sequence
  Human TREM1 mutant  27_37_47
AATKLTEEKYELKEGQTLDVKCDYTLEKFASSQKAWQAIRDGEMPKTAACTERPSKNSHPVQVGRIILEDYHDHGLLRVRMVNLQVEDSGLYQCVIYQPPKEPHMLFDRIRLVVTKG

SEQ ID NO: 194 - protein, 117 aa, Artificial Sequence
  Human TREM1 mutant  28_38_44
AATKLTEEKYELKEGQTLDVKCDYTLEKFASSQKAWQIARDGEMAKTLACTERPSKNSHPVQVGRIILEDYHDHGLLRVRMVNLQVEDSGLYQCVIYQPPKEPHMLFDRIRLVVTKG

SEQ ID NO: 195 - protein, 117 aa, Artificial Sequence
  Human TREM1 mutant  29_38_90
AATKLTEEKYELKEGQTLDVKCDYTLEKFASSQKAWQIARDGEMPKTLACTERPSKNSHPVQVGRIILEDYHDHGLLRVRMVNLQVEDSGAYQCVIYQPPKEPHMLFDRIRLVVTKG

SEQ ID NO: 196 - protein, 117 aa, Artificial Sequence
  Human TREM1 mutant  30_42_43
AATKLTEEKYELKEGQTLDVKCDYTLEKFASSQKAWQIIRDGAAPKTLACTERPSKNSHPVQVGRIILEDYHDHGLLRVRMVNLQVEDSGLYQCVIYQPPKEPHMLFDRIRLVVTKG

SEQ ID NO: 197 - protein, 117 aa, Artificial Sequence
  Human TREM1 mutant  31_42_45
AATKLTEEKYELKEGQTLDVKCDYTLEKFASSQKAWQIIRDGAMPATLACTERPSKNSHPVQVGRIILEDYHDHGLLRVRMVNLQVEDSGLYQCVIYQPPKEPHMLFDRIRLVVTKG

SEQ ID NO: 198 - protein, 117 aa, Artificial Sequence
  Human TREM1 mutant  32_43_44
AATKLTEEKYELKEGQTLDVKCDYTLEKFASSQKAWQIIRDGEAAKTLACTERPSKNSHPVQVGRIILEDYHDHGLLRVRMVNLQVEDSGLYQCVIYQPPKEPHMLFDRIRLVVTKG

SEQ ID NO: 199 - protein, 117 aa, Artificial Sequence
  Human TREM1 mutant  33_43_101
AATKLTEEKYELKEGQTLDVKCDYTLEKFASSQKAWQIIRDGEAPKTLACTERPSKNSHPVQVGRIILEDYHDHGLLRVRMVNLQVEDSGLYQCVIYQPPKAPHMLFDRIRLVVTKG

SEQ ID NO: 200 - protein, 117 aa, Artificial Sequence
  Human TREM1 mutant  34_44_45
AATKLTEEKYELKEGQTLDVKCDYTLEKFASSQKAWQIIRDGEMAATLACTERPSKNSHPVQVGRIILEDYHDHGLLRVRMVNLQVEDSGLYQCVIYQPPKEPHMLFDRIRLVVTKG

SEQ ID NO: 201 - protein, 117 aa, Artificial Sequence
  Human TREM1 mutant  35_45_47
AATKLTEEKYELKEGQTLDVKCDYTLEKFASSQKAWQIIRDGEMPATAACTERPSKNSHPVQVGRIILEDYHDHGLLRVRMVNLQVEDSGLYQCVIYQPPKEPHMLFDRIRLVVTKG

SEQ ID NO: 202 - protein, 117 aa, Artificial Sequence
  Human TREM1 mutant  36_47_65
AATKLTEEKYELKEGQTLDVKCDYTLEKFASSQKAWQIIRDGEMPKTAACTERPSKNSHPVQVGRAILEDYHDHGLLRVRMVNLQVEDSGLYQCVIYQPPKEPHMLFDRIRLVVTKG

SEQ ID NO: 203 - protein, 117 aa, Artificial Sequence
  Human TREM1 mutant  37_51_55
AATKLTEEKYELKEGQTLDVKCDYTLEKFASSQKAWQIIRDGEMPKTLACTARPSANSHPVQVGRIILEDYHDHGLLRVRMVNLQVEDSGLYQCVIYQPPKEPHMLFDRIRLVVTKG

SEQ ID NO: 204 - protein, 117 aa, Artificial Sequence
  Human TREM1 mutant  38_56_57
AATKLTEEKYELKEGQTLDVKCDYTLEKFASSQKAWQIIRDGEMPKTLACTERPSKAAHPVQVGRIILEDYHDHGLLRVRMVNLQVEDSGLYQCVIYQPPKEPHMLFDRIRLVVTKG

SEQ ID NO: 205 - protein, 117 aa, Artificial Sequence
  Human TREM1 mutant  39_58_59
AATKLTEEKYELKEGQTLDVKCDYTLEKFASSQKAWQIIRDGEMPKTLACTERPSKNSAAVQVGRIILEDYHDHGLLRVRMVNLQVEDSGLYQCVIYQPPKEPHMLFDRIRLVVTKG

SEQ ID NO: 206 - protein, 117 aa, Artificial Sequence
  Human TREM1 mutant  40_59_60
AATKLTEEKYELKEGQTLDVKCDYTLEKFASSQKAWQIIRDGEMPKTLACTERPSKNSHAAQVGRIILEDYHDHGLLRVRMVNLQVEDSGLYQCVIYQPPKEPHMLFDRIRLVVTKG

SEQ ID NO: 207 - protein, 117 aa, Artificial Sequence
  Human TREM1 mutant  41_59_61
AATKLTEEKYELKEGQTLDVKCDYTLEKFASSQKAWQIIRDGEMPKTLACTERPSKNSHAVAVGRIILEDYHDHGLLRVRMVNLQVEDSGLYQCVIYQPPKEPHMLFDRIRLVVTKG

SEQ ID NO: 208 - protein, 117 aa, Artificial Sequence
  Human TREM1 mutant  42_59_66
AATKLTEEKYELKEGQTLDVKCDYTLEKFASSQKAWQIIRDGEMPKTLACTERPSKNSHAVQVGRIALEDYHDHGLLRVRMVNLQVEDSGLYQCVIYQPPKEPHMLFDRIRLVVTKG

SEQ ID NO: 209 - protein, 117 aa, Artificial Sequence
  Human TREM1 mutant  43_60_61
AATKLTEEKYELKEGQTLDVKCDYTLEKFASSQKAWQIIRDGEMPKTLACTERPSKNSHPAAVGRIILEDYHDHGLLRVRMVNLQVEDSGLYQCVIYQPPKEPHMLFDRIRLVVTKG

SEQ ID NO: 210 - protein, 117 aa, Artificial Sequence
  Human TREM1 mutant  44_60_62
AATKLTEEKYELKEGQTLDVKCDYTLEKFASSQKAWQIIRDGEMPKTLACTERPSKNSHPAQAGRIILEDYHDHGLLRVRMVNLQVEDSGLYQCVIYQPPKEPHMLFDRIRLVVTKG

SEQ ID NO: 211 - protein, 117 aa, Artificial Sequence
  Human TREM1 mutant  45_61_62
AATKLTEEKYELKEGQTLDVKCDYTLEKFASSQKAWQIIRDGEMPKTLACTERPSKNSHPVAAGRIILEDYHDHGLLRVRMVNLQVEDSGLYQCVIYQPPKEPHMLFDRIRLVVTKG

SEQ ID NO: 212 - protein, 117 aa, Artificial Sequence
  Human TREM1 mutant  46_61_66
AATKLTEEKYELKEGQTLDVKCDYTLEKFASSQKAWQIIRDGEMPKTLACTERPSKNSHPVAVGRIALEDYHDHGLLRVRMVNLQVEDSGLYQCVIYQPPKEPHMLFDRIRLVVTKG

SEQ ID NO: 213 - protein, 117 aa, Artificial Sequence
  Human TREM1 mutant  47_62_65
AATKLTEEKYELKEGQTLDVKCDYTLEKFASSQKAWQIIRDGEMPKTLACTERPSKNSHPVQAGRAILEDYHDHGLLRVRMVNLQVEDSGLYQCVIYQPPKEPHMLFDRIRLVVTKG

SEQ ID NO: 214 - protein, 117 aa, Artificial Sequence
  Human TREM1 mutant  48_65_66
AATKLTEEKYELKEGQTLDVKCDYTLEKFASSQKAWQIIRDGEMPKTLACTERPSKNSHPVQVGRAALEDYHDHGLLRVRMVNLQVEDSGLYQCVIYQPPKEPHMLFDRIRLVVTKG

SEQ ID NO: 215 - protein, 117 aa, Artificial Sequence
  Human TREM1 mutant  49_66_81
AATKLTEEKYELKEGQTLDVKCDYTLEKFASSQKAWQIIRDGEMPKTLACTERPSKNSHPVQVGRIALEDYHDHGLLRVRMANLQVEDSGLYQCVIYQPPKEPHMLFDRIRLVVTKG

SEQ ID NO: 216 - protein, 117 aa, Artificial Sequence
  Human TREM1 mutant  50_72_73
AATKLTEEKYELKEGQTLDVKCDYTLEKFASSQKAWQIIRDGEMPKTLACTERPSKNSHPVQVGRIILEDYHAAGLLRVRMVNLQVEDSGLYQCVIYQPPKEPHMLFDRIRLVVTKG

SEQ ID NO: 217 - protein, 117 aa, Artificial Sequence
  Human TREM1 mutant  51_73_75
AATKLTEEKYELKEGQTLDVKCDYTLEKFASSQKAWQIIRDGEMPKTLACTERPSKNSHPVQVGRIILEDYHDAGALRVRMVNLQVEDSGLYQCVIYQPPKEPHMLFDRIRLVVTKG

SEQ ID NO: 218 - protein, 117 aa, Artificial Sequence
  Human TREM1 mutant  52_81_82
AATKLTEEKYELKEGQTLDVKCDYTLEKFASSQKAWQIIRDGEMPKTLACTERPSKNSHPVQVGRIILEDYHDHGLLRVRMAALQVEDSGLYQCVIYQPPKEPHMLFDRIRLVVTKG

SEQ ID NO: 219 - protein, 117 aa, Artificial Sequence
  Human TREM1 mutant  53_85_86
AATKLTEEKYELKEGQTLDVKCDYTLEKFASSQKAWQIIRDGEMPKTLACTERPSKNSHPVQVGRIILEDYHDHGLLRVRMVNLQAADSGLYQCVIYQPPKEPHMLFDRIRLVVTKG

SEQ ID NO: 220 - protein, 117 aa, Artificial Sequence
  Human TREM1 mutant  54_98_99
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQAAKEPHMLFDRIRLVVTKG

SEQ ID NO: 221 - protein, 117 aa, Artificial Sequence
  Human TREM1 mutant  55_99_100
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPAAEPHMLFDRIRLVVTKG

SEQ ID NO: 222 - protein, 117 aa, Artificial Sequence
  Human TREM1 mutant  56_100_101
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPAAPHMLFDRIRLVVTKG

SEQ ID NO: 223 - protein, 117 aa, Artificial Sequence
  Human TREM1 mutant  57_101_102
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKAAHMLFDRIRLVVTKG

SEQ ID NO: 224 - protein, 117 aa, Artificial Sequence
  Human TREM1 mutant  58_102_103
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEAAMLFDRIRLVVTKG

SEQ ID NO: 225 - protein, 117 aa, Artificial Sequence
  Human TREM1 mutant  59_103_104
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPAALFDRIRLVVTKG

SEQ ID NO: 226 - protein, 117 aa, Artificial Sequence
  Human TREM1 mutant  60_104_106
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALADRIRLVVTKG

SEQ ID NO: 227 - protein, 117 aa, Artificial Sequence
  Human TREM1 mutant  61_106_107
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLAARIRLVVTKG

SEQ ID NO: 228 - protein, 117 aa, Artificial Sequence
  Human TREM1 mutant  62_8_10_112
AATKLTEEAYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG

SEQ ID NO: 229 - protein, 117 aa, Artificial Sequence
  Human TREM1 mutant  63_37_39_40
AATKLTEEKYELKEGQTLDVKCDYTLEKFASSQKAWQAIAAGEMPKTLACTERPSKNSHPVQVGRIILEDYHDHGLLRVRMVNLQVEDSGLYQCVIYQPPKEPHMLFDRIRLVVTKG

SEQ ID NO: 230 - protein, 117 aa, Artificial Sequence
  Human TREM1 mutant  64_40
AATKLTEEKYELKEGQTLDVKCDYTLEKFASSQKAWQIIRAGEMPKTLACTERPSKNSHPVQVGRIILEDYHDHGLLRVRMVNLQVEDSGLYQCVIYQPPKEPHMLFDRIRLVVTKG

SEQ ID NO: 231 - protein, 117 aa, Artificial Sequence
  Human TREM1 mutant  65_112
AATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTKG

SEQ ID NO: 232 - protein, 116 aa, Artificial Sequence
  Human TREM1 WT 01_WT_1A
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 233 - protein, 116 aa, Artificial Sequence
  Human TREM1 mutant  02_1
AAKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 234 - protein, 116 aa, Artificial Sequence
  Human TREM1 mutant  03_2
ATALTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 235 - protein, 116 aa, Artificial Sequence
  Human TREM1 mutant  04_4
ATKLAEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 236 - protein, 116 aa, Artificial Sequence
  Human TREM1 mutant  05_5
ATKLTAEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 237 - protein, 116 aa, Artificial Sequence
  Human TREM1 mutant  06_6
ATKLTEAKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 238 - protein, 116 aa, Artificial Sequence
  Human TREM1 mutant  07_7
ATKLTEEAYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 239 - protein, 116 aa, Artificial Sequence
  Human TREM1 mutant  08_8
ATKLTEEKAELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 240 - protein, 116 aa, Artificial Sequence
  Human TREM1 mutant  09_9
ATKLTEEKYALKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 241 - protein, 116 aa, Artificial Sequence
  Human TREM1 mutant  10_11
ATKLTEEKYELAEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 242 - protein, 116 aa, Artificial Sequence
  Human TREM1 mutant  11_14
ATKLTEEKYELKEGATLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 243 - protein, 116 aa, Artificial Sequence
  Human TREM1 mutant  12_17
ATKLTEEKYELKEGQTLAVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 244 - protein, 116 aa, Artificial Sequence
  Human TREM1 mutant  13_19
ATKLTEEKYELKEGQTLDVACDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 245 - protein, 116 aa, Artificial Sequence
  Human TREM1 mutant  14_21
ATKLTEEKYELKEGQTLDVKCAYTLEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 246 - protein, 116 aa, Artificial Sequence
  Human TREM1 mutant  15_23
ATKLTEEKYELKEGQTLDVKCDYALEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 247 - protein, 116 aa, Artificial Sequence
  Human TREM1 mutant  16_24
ATKLTEEKYELKEGQTLDVKCDYTAEKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 248 - protein, 116 aa, Artificial Sequence
  Human TREM1 mutant  17_25
ATKLTEEKYELKEGQTLDVKCDYTLAKFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 249 - protein, 116 aa, Artificial Sequence
  Human TREM1 mutant  18_26
ATKLTEEKYELKEGQTLDVKCDYTLEAFASSQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 250 - protein, 116 aa, Artificial Sequence
  Human TREM1 mutant  19_29
ATKLTEEKYELKEGQTLDVKCDYTLEKFAASQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 251 - protein, 116 aa, Artificial Sequence
  Human TREM1 mutant  20_30
ATKLTEEKYELKEGQTLDVKCDYTLEKFASAQKAWQIIRDGEMPKTLACTERPSKNSHPVQVGRIILEDYHDHGLLRVRMVNLQVEDSGLYQCVIYQPPKEPHMLFDRIRLVVTLE

SEQ ID NO: 252 - protein, 116 aa, Artificial Sequence
  Human TREM1 mutant  21_38
ATKLTEEKYELKEGQTLDVKCDYTLEKFASSQKAWQIIADGEMPKTLACTERPSKNSHPVQVGRIILEDYHDHGLLRVRMVNLQVEDSGLYQCVIYQPPKEPHMLFDRIRLVVTLE

SEQ ID NO: 253 - protein, 116 aa, Artificial Sequence
  Human TREM1 mutant  22_39
ATKLTEEKYELKEGQTLDVKCDYTLEKFASSQKAWQIIRAGEMPKTLACTERPSKNSHPVQVGRIILEDYHDHGLLRVRMVNLQVEDSGLYQCVIYQPPKEPHMLFDRIRLVVTLE

SEQ ID NO: 254 - protein, 116 aa, Artificial Sequence
  Human TREM1 mutant  23_41
ATKLTEEKYELKEGQTLDVKCDYTLEKFASSQKAWQIIRDGAMPKTLACTERPSKNSHPVQVGRIILEDYHDHGLLRVRMVNLQVEDSGLYQCVIYQPPKEPHMLFDRIRLVVTLE

SEQ ID NO: 255 - protein, 116 aa, Artificial Sequence
  Human TREM1 mutant  24_42
ATKLTEEKYELKEGQTLDVKCDYTLEKFASSQKAWQIIRDGEAPKTLACTERPSKNSHPVQVGRIILEDYHDHGLLRVRMVNLQVEDSGLYQCVIYQPPKEPHMLFDRIRLVVTLE

SEQ ID NO: 256 - protein, 116 aa, Artificial Sequence
  Human TREM1 mutant  25_44
ATKLTEEKYELKEGQTLDVKCDYTLEKFASSQKAWQIIRDGEMPATLACTERPSKNSHPVQVGRIILEDYHDHGLLRVRMVNLQVEDSGLYQCVIYQPPKEPHMLFDRIRLVVTLE

SEQ ID NO: 257 - protein, 116 aa, Artificial Sequence
  Human TREM1 mutant  26_45
ATKLTEEKYELKEGQTLDVKCDYTLEKFASSQKAWQIIRDGEMPKALACTERPSKNSHPVQVGRIILEDYHDHGLLRVRMVNLQVEDSGLYQCVIYQPPKEPHMLFDRIRLVVTLE

SEQ ID NO: 258 - protein, 116 aa, Artificial Sequence
  Human TREM1 mutant  27_50
ATKLTEEKYELKEGQTLDVKCDYTLEKFASSQKAWQIIRDGEMPKTLACTARPSKNSHPVQVGRIILEDYHDHGLLRVRMVNLQVEDSGLYQCVIYQPPKEPHMLFDRIRLVVTLE

SEQ ID NO: 259 - protein, 116 aa, Artificial Sequence
  Human TREM1 mutant  28_51
ATKLTEEKYELKEGQTLDVKCDYTLEKFASSQKAWQIIRDGEMPKTLACTEAPSKNSHPVQVGRIILEDYHDHGLLRVRMVNLQVEDSGLYQCVIYQPPKEPHMLFDRIRLVVTLE

SEQ ID NO: 260 - protein, 116 aa, Artificial Sequence
  Human TREM1 mutant  29_52
ATKLTEEKYELKEGQTLDVKCDYTLEKFASSQKAWQIIRDGEMPKTLACTERASKNSHPVQVGRIILEDYHDHGLLRVRMVNLQVEDSGLYQCVIYQPPKEPHMLFDRIRLVVTLE

SEQ ID NO: 261 - protein, 116 aa, Artificial Sequence
  Human TREM1 mutant  30_54
ATKLTEEKYELKEGQTLDVKCDYTLEKFASSQKAWQIIRDGEMPKTLACTERPSANSHPVQVGRIILEDYHDHGLLRVRMVNLQVEDSGLYQCVIYQPPKEPHMLFDRIRLVVTLE

SEQ ID NO: 262 - protein, 116 aa, Artificial Sequence
  Human TREM1 mutant  31_55
ATKLTEEKYELKEGQTLDVKCDYTLEKFASSQKAWQIIRDGEMPKTLACTERPSKASHPVQVGRIILEDYHDHGLLRVRMVNLQVEDSGLYQCVIYQPPKEPHMLFDRIRLVVTLE

SEQ ID NO: 263 - protein, 116 aa, Artificial Sequence
  Human TREM1 mutant  32_56
ATKLTEEKYELKEGQTLDVKCDYTLEKFASSQKAWQIIRDGEMPKTLACTERPSKNAHPVQVGRIILEDYHDHGLLRVRMVNLQVEDSGLYQCVIYQPPKEPHMLFDRIRLVVTLE

SEQ ID NO: 264 - protein, 116 aa, Artificial Sequence
  Human TREM1 mutant  33_57
ATKLTEEKYELKEGQTLDVKCDYTLEKFASSQKAWQIIRDGEMPKTLACTERPSKNSAPVQVGRIILEDYHDHGLLRVRMVNLQVEDSGLYQCVIYQPPKEPHMLFDRIRLVVTLE

SEQ ID NO: 265 - protein, 116 aa, Artificial Sequence
  Human TREM1 mutant  34_58
ATKLTEEKYELKEGQTLDVKCDYTLEKFASSQKAWQIIRDGEMPKTLACTERPSKNSHAVQVGRIILEDYHDHGLLRVRMVNLQVEDSGLYQCVIYQPPKEPHMLFDRIRLVVTLE

SEQ ID NO: 266 - protein, 116 aa, Artificial Sequence
  Human TREM1 mutant  35_59
ATKLTEEKYELKEGQTLDVKCDYTLEKFASSQKAWQIIRDGEMPKTLACTERPSKNSHPAQVGRIILEDYHDHGLLRVRMVNLQVEDSGLYQCVIYQPPKEPHMLFDRIRLVVTLE

SEQ ID NO: 267 - protein, 116 aa, Artificial Sequence
  Human TREM1 mutant  36_60
ATKLTEEKYELKEGQTLDVKCDYTLEKFASSQKAWQIIRDGEMPKTLACTERPSKNSHPVAVGRIILEDYHDHGLLRVRMVNLQVEDSGLYQCVIYQPPKEPHMLFDRIRLVVTLE

SEQ ID NO: 268 - protein, 116 aa, Artificial Sequence
  Human TREM1 mutant  37_61
ATKLTEEKYELKEGQTLDVKCDYTLEKFASSQKAWQIIRDGEMPKTLACTERPSKNSHPVQAGRIILEDYHDHGLLRVRMVNLQVEDSGLYQCVIYQPPKEPHMLFDRIRLVVTLE

SEQ ID NO: 269 - protein, 116 aa, Artificial Sequence
  Human TREM1 mutant  38_63
ATKLTEEKYELKEGQTLDVKCDYTLEKFASSQKAWQIIRDGEMPKTLACTERPSKNSHPVQVGAIILEDYHDHGLLRVRMVNLQVEDSGLYQCVIYQPPKEPHMLFDRIRLVVTLE

SEQ ID NO: 270 - protein, 116 aa, Artificial Sequence
  Human TREM1 mutant  39_65
ATKLTEEKYELKEGQTLDVKCDYTLEKFASSQKAWQIIRDGEMPKTLACTERPSKNSHPVQVGRIALEDYHDHGLLRVRMVNLQVEDSGLYQCVIYQPPKEPHMLFDRIRLVVTLE

SEQ ID NO: 271 - protein, 116 aa, Artificial Sequence
  Human TREM1 mutant  40_71
ATKLTEEKYELKEGQTLDVKCDYTLEKFASSQKAWQIIRDGEMPKTLACTERPSKNSHPVQVGRIILEDYHAHGLLRVRMVNLQVEDSGLYQCVIYQPPKEPHMLFDRIRLVVTLE

SEQ ID NO: 272 - protein, 116 aa, Artificial Sequence
  Human TREM1 mutant  41_72
ATKLTEEKYELKEGQTLDVKCDYTLEKFASSQKAWQIIRDGEMPKTLACTERPSKNSHPVQVGRIILEDYHDAGLLRVRMVNLQVEDSGLYQCVIYQPPKEPHMLFDRIRLVVTLE

SEQ ID NO: 273 - protein, 116 aa, Artificial Sequence
  Human TREM1 mutant  42_78
ATKLTEEKYELKEGQTLDVKCDYTLEKFASSQKAWQIIRDGEMPKTLACTERPSKNSHPVQVGRIILEDYHDHGLLRVAMVNLQVEDSGLYQCVIYQPPKEPHMLFDRIRLVVTLE

SEQ ID NO: 274 - protein, 116 aa, Artificial Sequence
  Human TREM1 mutant  43_80
ATKLTEEKYELKEGQTLDVKCDYTLEKFASSQKAWQIIRDGEMPKTLACTERPSKNSHPVQVGRIILEDYHDHGLLRVRMANLQVEDSGLYQCVIYQPPKEPHMLFDRIRLVVTLE

SEQ ID NO: 275 - protein, 116 aa, Artificial Sequence
  Human TREM1 mutant  44_81
ATKLTEEKYELKEGQTLDVKCDYTLEKFASSQKAWQIIRDGEMPKTLACTERPSKNSHPVQVGRIILEDYHDHGLLRVRMVALQVEDSGLYQCVIYQPPKEPHMLFDRIRLVVTLE

SEQ ID NO: 276 - protein, 116 aa, Artificial Sequence
  Human TREM1 mutant  45_83
ATKLTEEKYELKEGQTLDVKCDYTLEKFASSQKAWQIIRDGEMPKTLACTERPSKNSHPVQVGRIILEDYHDHGLLRVRMVNLAVEDSGLYQCVIYQPPKEPHMLFDRIRLVVTLE

SEQ ID NO: 277 - protein, 116 aa, Artificial Sequence
  Human TREM1 mutant  46_84
ATKLTEEKYELKEGQTLDVKCDYTLEKFASSQKAWQIIRDGEMPKTLACTERPSKNSHPVQVGRIILEDYHDHGLLRVRMVNLQAEDSGLYQCVIYQPPKEPHMLFDRIRLVVTLE

SEQ ID NO: 278 - protein, 116 aa, Artificial Sequence
  Human TREM1 mutant  47_85
ATKLTEEKYELKEGQTLDVKCDYTLEKFASSQKAWQIIRDGEMPKTLACTERPSKNSHPVQVGRIILEDYHDHGLLRVRMVNLQVADSGLYQCVIYQPPKEPHMLFDRIRLVVTLE

SEQ ID NO: 279 - protein, 116 aa, Artificial Sequence
  Human TREM1 mutant  48_87
ATKLTEEKYELKEGQTLDVKCDYTLEKFASSQKAWQIIRDGEMPKTLACTERPSKNSHPVQVGRIILEDYHDHGLLRVRMVNLQVEDAGLYQCVIYQPPKEPHMLFDRIRLVVTLE

SEQ ID NO: 280 - protein, 116 aa, Artificial Sequence
  Human TREM1 mutant  49_95
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIAQPPKEPHMLFDRIRLVVTLE

SEQ ID NO: 281 - protein, 116 aa, Artificial Sequence
  Human TREM1 mutant  50_97
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQAPKEPHMLFDRIRLVVTLE

SEQ ID NO: 282 - protein, 116 aa, Artificial Sequence
  Human TREM1 mutant  51_98
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPAKEPHMLFDRIRLVVTLE

SEQ ID NO: 283 - protein, 116 aa, Artificial Sequence
  Human TREM1 mutant  52_99
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPAEPHMLFDRIRLVVTLE

SEQ ID NO: 284 - protein, 116 aa, Artificial Sequence
  Human TREM1 mutant  53_100
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKAPHMLFDRIRLVVTLE

SEQ ID NO: 285 - protein, 116 aa, Artificial Sequence
  Human TREM1 mutant  54_101
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEAHMLFDRIRLVVTLE

SEQ ID NO: 286 - protein, 116 aa, Artificial Sequence
  Human TREM1 mutant  55_102
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPAMLFDRIRLVVTLE

SEQ ID NO: 287 - protein, 116 aa, Artificial Sequence
  Human TREM1 mutant  56_103
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHALFDRIRLVVTLE

SEQ ID NO: 288 - protein, 116 aa, Artificial Sequence
  Human TREM1 mutant  57_104
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMAFDRIRLVVTLE

SEQ ID NO: 289 - protein, 116 aa, Artificial Sequence
  Human TREM1 mutant  58_106
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFARIRLVVTLE

SEQ ID NO: 290 - protein, 116 aa, Artificial Sequence
  Human TREM1 mutant  59_109
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIALVVTLE

SEQ ID NO: 291 - protein, 116 aa, Artificial Sequence
  Human TREM1 mutant  60_111
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLAVTLE

SEQ ID NO: 292 - protein, 116 aa, Artificial Sequence
  Human TREM1 mutant  61_113
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVALE

SEQ ID NO: 293 - protein, 116 aa, Artificial Sequence
  Human TREM1 mutant  62_114
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTAE

SEQ ID NO: 294 - protein, 116 aa, Artificial Sequence
  Human TREM1 mutant  63_115
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTLA

(19) United States
(12) Patent Application Publication
(10) Pub. No.: US 2024/0254224 A1
(43) Pub. Date: Aug. 1, 2024

(54) ANTIBODIES

(71) Applicant: UCB BIOPHARMA SRL, Brussels (BE)

(72) Inventors: Daniel John LIGHTWOOD, Slough, Berkshire (GB); Irena KADIU, Brussels (BE); Pallavi BHATTA, Slough, Berkshire (GB); Anastasios SPILIOTOPOULOS, Slough, Berkshire (GB); Peter Charles ELLIOTT, Slough, Berkshire (GB); James Martin KEANEY, Brussels (BE); Silvia L. DELKER, Brussels (BE); Jan ABENDROTH, Brussels (BE)

(21) Appl. No.: 18/558,686

(22) PCT Filed: May 2, 2022
(86) PCT No.: PCT/EP2022/061661

§ 371 Date: Nov. 2, 2023

Related U.S. Application Data
(60) Provisional application No. 63/183,280, filed on May 3, 2021.

(51) Int. Cl. C07K 16/28; A61K 39/00; A61P 25/28

(52) U.S. Cl. CPC C07K 16/2803; A61P 25/28; A61K 2039/505; C07K 2317/24; C07K 2317/33; C07K 2317/34; C07K 2317/55; C07K 2317/565; C07K 2317/76; C07K 2317/92

(57) ABSTRACT

The present invention relates to antibodies binding to TREM1 and inhibiting its interaction with one or more of its natural ligands. Specific examples of such antibodies are provided. The therapeutic uses of the antibodies and methods of generating such are also provided.

### FIELD OF THE INVENTION

The present invention relates to anti-TREM1 antibodies and their use in the treatment of neurological disorders, and more particularly, for the treatment of amyotrophic lateral sclerosis (ALS) and Alzheimer's disease BACKGROUND

Triggering receptors expressed on myeloid cells (TREM) are receptors including immune-activating and -inhibitory isoforms encoded by an MHC gene cluster mapping to human chromosome 6p21 and mouse chromosome 17. TREMs are members of the immunoglobulin (Ig) superfamily, primarily expressed in cells of the myeloid lineage including monocytes, neutrophils, and dendritic cells in the periphery and microglia in the central nervous system (CNS). Triggering receptor expressed on myeloid cells-1 (TREM1), otherwise known as cluster of differentiation 354 is the first member of the TREM family to be identified and it has limited homology with other receptors of the Ig superfamily. TREM1 is a transmembrane glycoprotein with a single Ig-like domain, a transmembrane region with a (+) charged lysine residue interacting with a negatively charged aspartic acid on its signaling partner DAP12 and a short cytoplasmic tail that lacks any signaling domains.

TREM1 activation through interactions with its proposed ligand peptidoglycan recognition protein 1 (PGLYRP1), high mobility group B1 (HMGB1), soluble CD177, heat shock protein 70 (HSP70), extracellular cold-inducible RNA-binding protein (eCIRP) has been proposed to induce formation of an “head-to-tail” homodimer. Dimer crosslinking triggers the phosphorylation of the immune receptor tyrosine-based activating motif (ITAM) on the recruited DAP12, which enables signaling and function by providing a docking site for spleen tyrosine kinase (SYK) and its downstream signaling partners including zeta-chain-associated protein kinase 70 (ZAP70), casitas b-lineage lymphoma (Cbl), son of sevenless (SOS) and growth factor receptor binding protein 2 (GRB2). These interactions trigger downstream signal transduction through phosphatidylinositol 3-kinase (P13K), phospholipase-C-γ 2 (PLC-γ2) and the ERK pathways. These events are followed by calcium mobilization, activation of transcription factors including ETS-containing protein (ELK1), nuclear factor of activated T-cells (NFAT), AP1, c-fos, c-Jun and NF-κB. This pathway is shared with another member of the TREM family TREM2.

Unlike TREM1 which is clearly an immune activator TREM2 can act as both pro- and anti-inflammatory when binding to high and low affinity ligands respectively. Under homeostatic conditions TREM2 interaction with low affinity ligands keeps the pathway in check maintaining homeostasis (Konishi H., et al. Frontiers Cellular Neuroscience 2018).

In neuroinflammatory neurodegenerative conditions intracellular factors (among others TREM1 ligands) otherwise known as Damage Associated Molecular Patterns (DAMPs) are spilled from dying neurons and activate surveilling microglia through TREM1 and other pattern recognition receptors. TREM1-DAMPs interaction overrides TREM2 activity resulting in microglia/innate immune activation, direct neurotoxicity and destruction of synaptic architecture through aberrant phagocytosis. Beyond its “Yin and Yang” dynamic with TREM2 in pathway regulation TREM1 carries unique and distinct functions as a potentiator of other key regulators of innate immune response including Toll-like (TLRs) and NOD-like receptor families. Amplification of these receptors occurs either through TREM1-induced overexpression of TLRs, their downstream nodes such as MYD88 and IKk or through direct cross-linking through TREM1 ligand complex formed between a TLR agonist and a TREM1 ligand as is the case with PGN (a TLR2/TLR4 stimulator) and PGLYRP1 (TREM1 ligand).

The consequence of TREM1 multi-pathway activation results in amplified innate immune/microglial pro-inflammatory responses including cytokine and chemokine release, upregulation of costimulatory molecules/antigen presentation and aberrant phagocytic activity. downstream (Buchon et al, 2000). These processes are a common denominator to the pathobiology in various neurodegenerative, neurodevelopmental and autoimmune central nervous system disorders. Human genetics including Genome Wide Association studies (GWAS) have implicated TREM2, several nodes downstream of TREM1/TREM2 pathway such as DAP12, Syk, PLCγ2 and TLRs as risk genes in various neurodegenerative disease.

U.S. Pat. No. 9,000,127 provides anti-TREM1 antibodies that disrupt the interaction of TREM1 with its ligand. The disclosed antibodies are provided for the treatment of individuals with an inflammatory disease, such as rheumatoid arthritis and inflammatory bowel disease.

WO 2017/152102 discloses antibodies that bind to a TREM1 protein and modulate or enhance one or more TREM1 activities.

### SUMMARY OF THE INVENTION

The present invention addresses the need for new treatments of neurological disorders by providing anti-TREM1 antibodies with the functional and structural properties as described herein.

In particular, the present invention provides an antibody that binds to human TREM1, comprising:
a light chain variable region comprising:
   a CDR-L1 comprising SEQ ID NO:11,
   a CDR-L2 comprising SEQ ID NO: 12, and
   a CDR-L3 comprising SEQ ID NO: 13;
and a heavy chain variable region comprising:
   a CDR-H1 comprising SEQ ID NO: 14,
   a CDR-H2 comprising SEQ ID NO: 15, and
   a CDR-H3 comprising SEQ ID NO: 16.

### BRIEF DESCRIPTION OF THE DRAWINGS

The present invention is described below by reference to the following drawings, in which:

FIG. 1 shows humanization of antibody 12172 light chain. Different variants generated for that chain are also shown. The CDR sequences are underlined.

FIG. 2 shows humanization of antibody 12172 heavy chain. Different variants generated for that chain are also shown. The CDR sequences are underlined.

FIGS. 3A and 3B show crystal structure of human TREM1 bound to PGLYRP1 and 12172 rabbit Fab. (3A) Crystal structure of human TREM1 bound to PGLYRP1. TREM1 residues with atoms within 4 Angstroms of any atom belonging to PGLYRP1 are highlighted in black. (3B) Crystal structure of human TREM1 bound to 12172 Rabbit Fab. TREM1 residues with atoms within 4 Angstroms of any atom belonging to 12172 are highlighted in black.

FIG. 4 shows thermal stability of different variants of 12172 antibody. Thermograms for 12172 gL2gH11 and 12172 gL6gH6 (hIgG4P and hIgG1 LALA) measured in a common pre-formulation storage buffer pH7.4.

FIG. 5 shows inhibition of TREM1-mediated release of TNF-α, IL-6 and IL-10 by 12172 gL2gH 11 hIgG4P from primary human monocytes.

FIG. 6 shows increase of the release of IL-1R antagonist from primary human monocytes by various 12172 variants and a reference antibody.

FIG. 7 shows the effects of 12172 gL2gH11 hIgG4P and 0318-IgG1.3f in increasing IL-1RA release from unstimulated primary human monocytes.

FIG. 8 shows efficacy of 12172 gL2gH11 hIgG4P on TNF-α and IL-6 release from healthy control and Alzheimer Disease (AD) PBMCs.

FIG. 9 shows efficacy of 12172 gL2gH11 hIgG4P on TNF-α and IL-6 release from healthy control and ALS PBMCs.

FIG. 10 shows efficacy of 12172 gL2gH11 hIgG4P on pro-inflammatory cytokine and chemokine release from ALS and AD PBMCs.

FIGS. 11A-11C show volcano plots showing Differentially Expressed Genes (DEGs) (considering an FDR of 0.05) following treatment of human monocytes with 12172 antibody variants (11A and 11B) or 0318-IgG1.3f (11C) and stimulation with TREM1 ligand complex (compared to isotype control).

FIGS. 12A-12C show volcano plots showing DEGs (considering an FDR of 0.05) following treatment of human monocytes with 12172 antibody variants (12A and 12) or 0318-IgG1.3f (12C) and stimulation with apoptotic human WPSC-derived motor neurons (compared to isotype control).

FIG. 13 shows that 12172 gL2gH11 hIgG4P does not impact E. coli clearance by human neutrophils and monocytes in vitro. The data is representative of 3 individual donors for 12172 gL2gH11 hIgG4P v Isotype and one donor including 0318-IgG1.3f(Ab 318) molecule for comparison. Statistical analysis, One-way ANOVA Dunnet post test was performed to compare anti-TREM antibodies against isotype control **** (p<0.0001)

FIG. 14 shows efficacy and potency of 12172 antibody variants in blocking SYK activation in hTREM1/hDAP-12 Flp-In 293 cells.

### DETAILED DESCRIPTION OF THE INVENTION

### Abbreviations

TABLE 1
Abbreviations used throughout the specification

| | |
|---|---|
| ADCC | antibody-dependent cellular cytotoxicity |
| CDC | complement dependent cytotoxicity |
| CDR | complementarity-determining region |
| CH1, CH2, CH3 | constant heavy domains 1, 2, 3 |
| CL | constant light domain |
| dsscFv | disulphide stabilised scFv |
| Fab | fragment antigen-binding |
| Fc | fragment crystallizable |
| FR1, FR2, FR3, FR4 | framework regions 1, 2, 3, 4 |
| Fv | variable domain fragment |
| HVR | hyper-variable region |
| KD | constant of dissociation |
| mAb | monoclonal antibody |
| scFv | single chain variable-fragment |
| VH | Heavy chain variable region |
| VHH | single domain antibody (or a camelid) |
| VL | variable light region |
| VNAR | variable domain of IgNAR |

TABLE 2
Amino acids abbreviations

| Abbreviation | 1 letter abbreviation | Amino acid name |
|---|---|---|
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |

### Definitions

The following terms are used throughout the specification.

The term “acceptor human framework” is used herein is a framework comprising the amino acid sequence of a light chain variable domain (VL) framework or a heavy chain variable domain (VH) framework derived from a human immunoglobulin framework or a human consensus framework. An acceptor human framework derived from a human immunoglobulin framework or a human consensus framework may comprise the same amino acid sequence thereof, or it may contain amino acid sequence changes.

The term “affinity” refers to the strength of all noncovalent interactions between an antibody thereof and the target protein. Unless indicated otherwise, as used herein, the term “binding affinity” refers to intrinsic binding affinity which reflects a 1:1 interaction between members of a binding pair (e.g., antibody and antigen). The affinity of a molecule for its binding partner can be generally represented by the dissociation constant (KD). Affinity can be measured by common methods known in the art, including those described herein.

The term “affinity matured” in the context of antibody refers to an antibody with one or more alterations in the hypervariable regions, compared to a parent antibody which does not possess such alterations, where such alterations resulting in an improvement in the affinity of the antibody for antigen.

The term “antibody” herein is used in the broadest sense and encompasses various antibody structures, including but not limited to monoclonal antibodies, polyclonal antibodies, and multi-specific antibodies as long as they exhibit the desired antigen-binding activity. The term antibody as used herein relates to whole (full-length) antibodies (i.e. comprising the elements of two heavy chains and two light chains) and functionally active fragments thereof (i.e., molecules that contain an antigen binding domain that specifically binds an antigen, also termed antibody fragments or antigen-binding fragments). Features described herein with respect to antibodies also apply to antibody fragments unless context dictates otherwise. The term “antibody” encompasses monovalent, i.e., antibodies comprising only one antigen binding domain (e.g. one-armed antibodies comprising a full-length heavy chain and a full-length light chain interconnected, also termed “half-antibody”), and multivalent antibodies, i. e. antibodies comprising more than one antigen binding domain, e.g bivalent.

The term “antibody binding to the same epitope as a reference antibody” refers to an antibody that blocks binding of the reference antibody to its antigen in a competition assay by 50% or more, and conversely, the reference antibody blocks binding of the antibody to its antigen in a competition assay by 50% or more.

The term “Antibody-dependent cellular cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells.

The term “antigen-binding fragment” as employed herein refers to functionally active antibody binding fragments including but not limited to Fab, modified Fab, Fab′, modified Fab′, F(ab′)2, Fv, single domain antibodies, scFv, Fv, bi, tri or tetra-valent antibodies, Bis-scFv, diabodies, triabodies, tetrabodies and epitope-binding fragments of any of the above (see for example Holliger and Hudson, 2005, Nature Biotech. 23(9): 1126-1136; Adair and Lawson, 2005, Drug Design Reviews—Online 2(3), 209-217). A “binding fragment” as employed herein refers to a fragment capable of binding a target peptide or antigen with sufficient affinity to characterize the fragment as specific for the peptide or antigen.

The term “antibody variant” refers to a polypeptide, for example, an antibody possessing the desired characteristics described herein and comprising a VH and/or a VL that has at least about 80% amino acid sequence identity with a VH and/or a VL of the reference antibody. Such antibody variants include, for instance, antibodies wherein one or more amino acid residues are added to or deleted from the VH and/or a VL domain. Ordinarily, an antibody variant will have at least about 80% amino acid sequence identity, alternatively at least about 85%, 90%, 95%, 96%, 97%, 98%, or 99% amino acid sequence identity, to an antibody described herein. Optionally, variant antibodies will have no more than one conservative amino acid substitution as compared to an antibody sequence provided herein, alternatively no more than about any of 2, 3, 4, 5, 6, 7, 8, 9, or 10 conservative amino acid substitutions as compared to an antibody sequence provided herein. In embodiments, an “antibody variant” refers to an antibody or antigen-binding fragment thereof comprising a VH and/or a VL wherein the non-CDR regions of the antibody or antigen-binding fragment thereof has at least about 85%, 90%, 95%, 96%, 97%, 98%, or 99% amino acid sequence identity, to an antibody described herein.

The term “antigen-binding domain” as employed herein refers to a portion of the antibody, which comprises a part or the whole of one or more variable domains, for example a part or the whole of a pair of variable domains VH and VL, that interact specifically with the target antigen. A binding domain may comprise a single domain antibody. Each binding domain may be monovalent. Each binding domain may comprise no more than one VH and one VL.

The term “bispecific” or “bispecific antibody” as employed herein refers to an antibody with two antigen specificities.

The term “complementarity determining regions” or “CDRs” refers to generally, antibodies comprise six CDRs: three in the VH (H1, H2, H3), and three in the VL (L1, L2, L3). The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system. Unless indicated otherwise, CDR residues and other residues in the variable domain (e.g., FR residues) are numbered herein according to Kabat.

The term “chimeric” antibody refers to an antibody in which the variable domain (or at least a portion thereof) of the heavy and/or light chain is derived from a particular source or species, while the remainder of the heavy and/or light chain (i.e. the constant domains) is derived from a different source or species. (Morrison; PNAS 81, 6851 (1984)). Chimeric antibodies can for instance comprise non-human variable domains and human constant domains. Chimeric antibodies are typically produced using recombinant DNA methods. A subcategory of “chimeric antibodies” is “humanized antibodies”.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2.

The heavy chain constant domains that correspond to the different classes of immunoglobulins are called α, δ, ε, γ, and, respectively.

The term “complement-dependent cytotoxicity”, or “CDC” refers to a mechanism for inducing cell death in which an Fc effector domain of a target-bound antibody binds and activates complement component C1q which in turn activates the complement cascade leading to target cell death.

The terms “constant domain(s)” or “constant region”, as used herein are used interchangeably to refer to the domain(s) of an antibody which is outside the variable regions. The constant domains are identical in all antibodies of the same isotype but are different from one isotype to another. Typically, the constant region of a heavy chain is formed, from N to C terminal, by CH1-hinge —CH2-CH3-optionally CH4, comprising three or four constant domains.

The term “competing antibody” or “cross-competing antibody” shall be interpreted as meaning that the claimed antibody binds to either (i) the same position on the antigen to which the reference antibody binds, or (ii) a position on the antigen where the antibody sterically hinders the binding of the reference antibody to the antigen.

The term “Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The term “derived from” in the context of generating variable sequences refers to the fact that the sequence employed or a sequence highly similar to the sequence employed was obtained from the original genetic material, such as the light or heavy chain of an antibody.

The term “diabody” as employed herein refers to two Fv pairs, a first VH/VL pair and a further VH/VL pair which have two inter-Fv linkers, such that the VH of a first Fv is linked to the VL of the second Fv and the VL of the first Fv is linked to the VH of the second Fv.

The term “DiFab” as employed herein refers to two Fab molecules linked via their C-terminus of the heavy chains.

The term “DiFab′” as employed herein refers to two Fab′ molecules linked via one or more disulfide bonds in the hinge region thereof.

The term “dsscFv” or “disulphide-stabilised single chain variable fragment” as employed herein refer to a single chain variable fragment which is stabilised by a peptide linker between the VH and VL variable domain and also includes an inter-domain disulphide bond between VH and VL. (see for example, Weatherill et al., Protein Engineering, Design & Selection, 25 (321-329), 2012, WO2007109254.

The term “DVD-Ig” (also known as dual V domain IgG) refers to a full-length antibody with 4 additional variable domains, one on the N-terminus of each heavy and each light chain.

The term “effector functions” refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), phagocytosis, down regulation of cell surface receptors (e.g. B cell receptor), and B cell activation.

The term “effector molecule” as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

The term “epitope” or “binding site” in the context of antibodies refer to a site (or a part) on an antigen to which the paratope of an antibody binds or recognizes. Epitopes can be formed both from contiguous amino acids (also often called “linear epitopes”) or noncontiguous amino acids formed by tertiary folding of a protein (often called “conformational epitopes”). Epitopes formed from contiguous amino acids are typically retained on exposure to denaturing solvents whereas epitopes formed by folding are typically lost on treatment with denaturing solvents. An epitope typically includes at least 3, and more usually, at least 5-10 amino acids in a unique spatial conformation. Epitopes usually consist of chemically active surface groups of molecules such as amino acids, sugar side chains and usually have specific 3D structural and charge characteristics.

The “EU index” or “EU index as in Kabat” or “EU numbering scheme” refers to the numbering of the EU antibody (Edelman et al., 1969, Proc Natl Acad Sci USA 63:78-85). Such is generally used when referring to a residue in an antibody heavy chain constant region (e.g., as reported in Kabat et al.). Unless stated otherwise, the EU numbering scheme is used to refer to residues in antibody heavy chain constant regions described herein.

The term “Fab” refers to as used herein refers to an antibody fragment comprising a light chain fragment comprising a VL (variable light) domain and a constant domain of a light chain (CL), and a VH (variable heavy) domain and a first constant domain (CH1) of a heavy chain. Dimers of a Fab′ according to the present disclosure create a F(ab′)2 where, for example, dimerization may be through the hinge.

The term “Fab′-Fv” as employed herein is similar to FabFv, wherein the Fab portion is replaced by a Fab′. The format may be provided as a PEGylated version thereof.

The term “Fab′-scFv” as employed herein is a Fab′ molecule with a scFv appended on the C-terminal of the light or heavy chain.

The term “Fab-dsFv” as employed herein refers to a FabFv wherein an intra-Fv disulfide bond stabilises the appended C-terminal variable regions. The format may be provided as a PEGylated version thereof.

The term “Fab-Fv” as employed herein refers to a Fab fragment with a variable region appended to the C-terminal of each of the following, the CH1 of the heavy chain and CL of the light chain. The format may be provided as a PEGylated version thereof.

The term “Fab-scFv” as employed herein is a Fab molecule with a scFv appended on the C-terminal of the light or heavy chain.

The term “Fc”, “Fc fragment”, and “Fe region” are used interchangeably to refer to the C-terminal region of an antibody comprising the constant region of an antibody excluding the first constant region immunoglobulin domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index. In the context of human IgG1, the lower hinge refers to positions 226-236, the CH2 domain refers to positions 237-340 and the CH3 domain refers to positions 341-447 according to the EU index. The corresponding Fc region of other immunoglobulins can be identified by sequence alignments.

The term “Framework” or “FR” refers to variable domain residues other than hypervariable region residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “full length antibody” used herein to refer to an antibody having a structure substantially similar to a native antibody structure or having heavy chains that contain an Fc region as defined herein. Each light chain is comprised of a light chain variable region (abbreviated herein as VL) and a light chain constant region (CL). Each heavy chain is comprised of a heavy variable region (abbreviated herein as VH) and a heavy chain constant region (CH) constituted of three constant domains CH1, CH2 and CH3, or four constant domains CH1, CH2, CH3 and CH4, depending on the Ig class. The constant regions of the antibodies may mediate the binding of the immunoglobulin to host tissues or factors, including various cells of the immune system (e.g., effector cells) and the first component (C1q) of the classical complement system.

The term “Fv” refers to two variable domains of full length antibodies, for example co-operative variable domains, such as a cognate pair or affinity matured variable domains, i.e. a VH and VL pair.

The term “highly similar” as employed in the context of amino-acid sequences is intended to refer to an amino acid sequence which over its full length is 95% similar or more, such as 96, 97, 98 or 99% similar.

The term “human antibody” refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “human consensus framework” refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al.

In some embodiments, for the VH, the subgroup is subgroup IV as in Kabat et al.

The term “humanized” antibody refers to an antibody comprising amino acid residues from non-human HVRs and amino acid residues from human FRs. Typically the heavy and/or light chain contains one or more CDRs (including, if desired, one or more modified CDRs) from a donor antibody (e.g. a non-human antibody such as a murine or rabbit monoclonal antibody) and is grafted into a heavy and/or light chain variable region framework of an acceptor antibody (a human antibody) (see e.g. Vaughan et al, Nature Biotechnology, 16, 535-539, 1998). The advantage of such humanized antibodies is to reduce immunogenicity to humans, while retaining the specificity and affinity of the parental non-human antibody. Rather than the entire CDR being transferred, only one or more of the specificity determining residues from any one of the CDRs described herein above can be transferred to the human antibody framework (see e.g., Kashmiri et al., 2005, Methods, 36, 25-34). A “humanized” antibody refers to a chimeric antibody comprising amino acid residues from non-human HVRs and amino acid residues from human FRs. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”).

The term “IC50” as used herein refers to the half maximal inhibitory concentration which is a measure of the effectiveness of a substance, such as an antibody, in inhibiting a specific biological or biochemical function. The IC50 is a quantitative measure which indicates how much of a particular substance is needed to inhibit a given biological process by 50%.

The “identity” between amino acids in the sequence indicates that at any particular position in the aligned sequences, the amino acid residue is identical between the sequences.

The term “IgG-scFv” as employed herein is a full-length antibody with a scFv on the C-terminal of each of the heavy chains or each of the light chains.

The term “IgG-V” as employed herein is a full-length antibody with a variable domain on the C-terminal of each of the heavy chains or each of the light chains.

The term “IgG1 LALA” or “hIgG1 LALA” refers mutant of the wild-type human IgG1 isoform in which amino acid substitutions L234A/L235A in the constant region of an IgG1 have been introduced.

The term “IgG4P” or “hIgG4P” refers to a mutant of the wild-type human IgG4 isoform in which amino acid 228 (according to EU numbering) is replaced by proline, as described for example in Angal et al., Molecular Immunology, 1993, 30 (1), 105-108.

The term “isolated” means, throughout this specification, that the antibody, or polynucleotide, as the case may be, exists in a physical milieu distinct from that in which it may occur in nature. The term “isolated” nucleic acid refers to a nucleic acid molecule that has been isolated from its natural environment or that has been synthetically created. An isolated nucleic acid may comprise synthetic DNA, for instance produced by chemical processing, cDNA, genomic DNA or any combination thereof.

The term “Kabat residue designations” or “Kabat” refer to the residue numbering scheme commonly used for antibodies. Such do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. For details see Kabat et al., Sequences of Proteins of Immunological Interest, 5th Ed. Public Health Service, National Institutes of Health, Bethesda, MD (1991). Unless indicated otherwise, Kabat numbering is used throughout the specification

The term “KD” as used herein refers to the constant of dissociation which is obtained from the ratio of Kd to K_{d} (i.e. Kd/Ka) and is expressed as a molar concentration (M). Kd and K_{d} refers to the dissociation rate and association rate, respectively, of a particular antigen-antibody interaction. KD values for antibodies can be determined using methods well established in the art.

The term “monoclonal antibody” (or “mAb”) refers to an antibody obtained from a population of substantially homogeneous antibodies, i.e. each individual of a monoclonal antibody preparation are identical except for possible mutations (e.g., naturally occurring mutations), that may be present in minor amounts. Certain differences in the protein sequences linked to post-translational modifications (for example, cleavage of the heavy chain C-terminal lysine, deamidation of asparagine residues and/or isomerisation of aspartate residues) may nevertheless exist between the various different antibody molecules present in the composition. Contrary to polyclonal antibody preparations, each monoclonal antibody of a monoclonal antibody preparation is directed against a single determinant on an antigen.

The term “multi-paratopic antibody” as employed herein refers to an antibody as described herein which comprises two or more distinct paratopes, which interact with different epitopes either from the same antigen or from two different antigens. Multi-paratopic antibodies described herein may be biparatopic, triparatopic, tetraparatopic.

The term “multispecific” or “multi-specific antibody” as employed herein refers to an antibody as described herein which has at least two binding domains, i.e. two or more binding domains, for example two or three binding domains, wherein the at least two binding domains independently bind two different antigens or two different epitopes on the same antigen. Multi-specific antibodies are generally monovalent for each specificity (antigen). Multi-specific antibodies described herein encompass monovalent and multivalent, e.g. bivalent, trivalent, tetravalent multi-specific antibodies.

For example, an antibody may comprise a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibodies are described in WO2015/197772.

The term “neutralizing” (or “neutralize”) in the context of antibodies describes an antibody that is capable of inhibiting or attenuating the biological signaling activity of its target (target protein).

The term “paratope” refers to a region of an antibody which recognizes and binds to an antigen.

The term “percent (%) sequence identity (or similarity)” with respect to the polypeptide and antibody sequences is defined as the percentage of amino acid residues in a candidate sequence that are identical (or similar) to the amino acid residues in the polypeptide being compared, after aligning the sequences and introducing gaps, if necessary, to achieve the maximum percent sequence identity, and not considering any conservative substitutions as part of the sequence identity

A “pharmaceutically acceptable carrier” refers to an ingredient in a pharmaceutical formulation, other than an active ingredient, which is nontoxic to a subject. Pharmaceutically acceptable carriers include, but are not limited to, a buffer, excipient, stabilizer, or preservative.

The term “polyclonal antibody” refers to a mixture of different antibody molecules which bind to (or otherwise interact with) more than one epitope of an antigen

The term “prevent” in the context of antibodies is used herein interchangeably with the term “inhibit” and indicates the effect the antibodies according to the present invention have with respect to a particular biological process or molecular interaction.

The term “scDiabody” refers to a diabody comprising an intra-Fv linker, such that the molecule comprises three linkers and forms a normal scFv whose VH and VL terminals are each linked to a one of the variable regions of a further Fv pair.

The term “Scdiabody-CH3” as employed herein refers to two scdiabody molecules each linked, for example via a hinge to a CH3 domain.

The term “ScDiabody-Fc” as employed herein is two scdiabodies, wherein each one is appended to the N-terminus of a CH2 domain, for example via a hinge, of constant region fragment —CH2CH3.

The term “single chain variable fragment” or “scFv” as employed herein refers to a single chain variable fragment which is stabilised by a peptide linker between the VH and VL variable domains.

The term “ScFv-Fc-scFv” as employed herein refers to four scFvs, wherein one of each is appended to the N-terminus and the C-terminus of both the heavy chains of a CH2CH3 fragment.

The term “scFv-IgG” as employed herein is a full-length antibody with a scFv on the N-terminal of each of the heavy chains or each of the light chains.

The term “similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

The term “single domain antibody” as used herein refers to an antibody fragment consisting of a single monomeric variable domain. Examples of single domain antibodies include VH or VL or VHH or V-NAR.

The term “specific” as employed herein in the context of antibodies is intended to refer to an antibody that only recognizes the antigen to which it is specific or an antibody that has significantly higher binding affinity to the antigen to which it is specific compared to binding to antigens to which it is non-specific, for example at least 5, 6, 7, 8, 9, 10 times higher binding affinity.

The term “sterically blocking” or “sterically preventing” as employed herein is intended to refer to the means of blocking an interaction between first and second proteins by a third protein's binding to the first protein. The binding between the first and the third proteins prevents the second protein from binding to the first protein due to unfavorable van der Waals or electrostatic interactions between the second and third proteins.

The terms “subject” or “individual” in the context of the treatments and diagnosis generally refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). More specifically, the individual or subject is a human

The term “Tandem scFv” as employed herein refers to at least two scFvs linked via a single linker such that there is a single inter-Fv linker.

The term “Tandem scFv-Fc” as employed herein refers to at least two tandem scFvs, wherein each one is appended to the N-terminus of a CH2 domain, for example via a hinge, of constant region fragment —CH2CH3.

The term “target” or “antibody target” as used herein refers to target antigen to which the antibody binds.

The term “Tetrabody” as employed herein refers to a format similar to the diabody comprising fours Fvs and four inter-Fv linkers.

The term “therapeutically effective amount” refers to the amount of an antibody thereof that, when administered to a subject for treating a disease, is sufficient to produce such treatment for the disease. The therapeutically effective amount will vary depending on the antibody, the disease and its severity and the age, weight, etc., of the subject to be treated.

The term “tribody” (also referred to a Fab(scFv)₂) as employed herein refers to a Fab fragment with a first scFv appended to the C-terminal of the light chain and a second scFv appended to the C-terminal of the heavy the chain.

The term “trispecific or trispecific antibody” as employed herein refers to an antibody with three antigen binding specificities. For example, the antibody is an antibody with three antigen binding domains (trivalent), which independently bind three different antigens or three different epitopes on the same antigen, i.e. each binding domain is monovalent for each antigen. One of the examples of a trispecific antibody format is TrYbe.

The terms “prevent”, or “preventing” and the like, refer to obtaining a prophylactic effect in terms of completely or partially preventing a disease or symptom thereof Preventing thus encompasses stopping the disease from occurring in a subject who may be predisposed to the disease but has not yet been diagnosed as having the disease.

The terms “treatment”, “treating” and the like, refer to obtaining a desired pharmacologic and/or physiologic effect. The effect may be therapeutic in terms of a partial or complete cure for a disease and/or adverse effect attributable to the disease. Treatment thus encompasses (a) inhibiting the disease, i.e., arresting its development; and (b) relieving the disease, i.e., causing regression of the disease.

The term “TrYbe” as employed herein refers to a tribody comprising two dsscFvs. dsFab as employed herein refers to a Fab with an intra-variable region disulfide bond.

The term “variable region” or “variable domain” refers to the domain of an antibody heavy or light chain that is involved in binding the antibody to antigen. The variable domains of the heavy chain (VH) and light chain (VL) of a full length antibody generally have similar structures, with each domain comprising four conserved framework regions (FRs) and three CDRs. (See, e.g., Kindt et al. Kuby Immunology, 6th ed., W. H. Freeman and Co., page 91 (2007).) A single VH or VL domain may be sufficient to confer antigen-binding specificity. Each VH and VL is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody are referred as CDR-H1, CDR-H2 and CDR-H3 and in the light chain variable regions as CDR-L1, CDR-L2 and CDR-L3. They are numbered sequentially in the direction from the N-terminus to the C-terminus of each chain. CDRs are conventionally numbered according to a system devised by Kabat.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” The term “vector” includes “expression vectors”.

The term “VH” refers to the variable domain (or the sequence) of the heavy chain.

The term “V-IgG” as employed herein is a full-length antibody with a variable domain on the N-terminal of each of the heavy chains or each of the light chains.

The term “VL” refers to the variable domain (or the sequence) of the light chain.

### TREM1

The term “TREM1” refers to “triggering receptor expressed on myeloid cells 1” (also known as TREM-1, and CD354) refers to a receptor that is expressed on monocytes, macrophages, neutrophils and other types of cells. Primary ligand for TREM1 include peptidoglycan-recognition-protein 1 (PGLYRP1), which belongs to a family of peptidoglycan (PGN) binding proteins (PGRPs). The term “TREM1” includes any variants or isoforms of TREM1 which are naturally expressed by cells.

Three isoforms of human TREM1 have been identified. Isoform 1 (Accession No. NP 061113.1; SEQ ID NO: 1) consists of 234 amino acids and represents the canonical sequence. Isoform 2 (Accession No. NP 001229518.1; SEQ ID NO: 2) consists of 225 amino acids and differ from the canonical sequence at amino acid residues 201-234. The amino acid residues encode part of the transmembrane domain and the cytoplasmic domain. Isoform 3 (Accession No. NP 001229519; SEQ ID NO: 3) consists of 150 amino acids, and is soluble. It lacks amino acid residues 151-234, which encode the transmembrane domain, the cytoplasmic domain, and part of the extracellular domain. The amino acid residues 138-150 also differ from the canonical sequence described above.

### Method of Identifying Anti-TREM1 Antibodies

In order to identify antibodies that would interact with different amino-acid residues on TREM1 than PGLYRP1 ligand and neutralize one or more of TREM1 activities, a special screening and testing strategy had to be developed, that involves measurement of binding to TREM1 and functional properties of the test antibodies, as well high-throughput measurement of the structural aspects of the binding (the target epitope residues). By establishing TREM1 residues involved in the interaction with PGLYRP1, this method allows to perform rapid testing and select antibodies for further development that would bind to a different site on TREM1 than PGLYRP1. Such antibodies could provide additional benefits of preventing binding of other potential ligands interacting with a different site of TREM1.

Hence, a method of identifying an antibody that interacts with different amino-acid residues on TREM1 than PGLYRP1 and neutralizes activity of human TREM1 is provided herein, said method comprising:
a) immunizing an animal with cells transiently expressing human TREM1;
b) recovering B cells from said animal;
c) selecting the antibodies produced by said B cells based on their ability to:
   i. bind to human TREM1 with affinity of at least 1 nM; and
   ii. block PGLYRP1-mediated signaling in the THP1 monocyte TREM1/DAP12 NF-κB Luciferase reporter cell assay; and
   iii. bind to a different site on human TREM1 than PGLYRP1.

In order to identify antibodies that bind to a different site than PGLYRP1, a method using arrays of mutant TREM1 proteins has been developed that allows rapid testing of the binding sites (residues of TREM1 involved in interaction with a test antibody) on TREM1 protein. The same method is used to determine the binding site of PGLYRP1 ligand. Such method of identifying amino-acid residues on TREM1 that form a binding site of a test antibody (or PGLYRP1), comprises:
a) obtaining 3D structure information for TREM1;
b) identifying, using obtained 3D structural data, the amino-acid residues which are within the accessible surface area;
c) for each of the identified amino-acids selecting 1 or 2 amino-acids which are within a predetermined distance from the identified amino-acid and are within the accessible surface area, whereby such combination of amino-acid residues forms a patch of 2 or 3 amino acids (patch);
d) selecting, from the large number of generated possible patches, a set of representative patches that cover the majority of TREM1's accessible surface area, while minimizing the number of patches likely to cause TREM1 protein to misfold;
e) producing a set of mutant proteins, wherein each of the mutant proteins comprises a mutated sequence of the target protein, wherein each of the mutated sequences comprises a single mutated patch of amino acids identified in step (c), and wherein each of the amino acids of the patch is substituted by another amino-acid;
f) measuring binding properties of each of the mutant proteins; and
g) identifying the patches that demonstrate decreased binding properties of the antibody to corresponding mutant TREM1 protein comprising such patch, wherein the residues in such patches are identified as being a part of a binding site of the antibody.

In a preferred embodiment, an antibody is selected if it interacts with the residues E26, E27, K28, Y29, E30, L31, K32 and Q35 of human TREM1 (where the numbering is according to SEQ ID NO: 1).

In order to identify the amino-acid residues for producing mutant versions of TREM1, 3D structure data needs to be obtained for TREM1. Such data is available in the form of a PDB structure (PDB code: 1SMO, chain A). Alternatively, such structural data can be obtained using the techniques known to the skilled person. Such techniques include X-ray analysis or NMR data. Preferably, such 3D data is a of sufficient spatial resolution to allow identification of the target residues.

In particular, the pre-determined distance between the residues of each patch is 4, 5, 6, or 7 Å. Preferably, such distance is 6 Å. Preferably, alanines and glycines are not selected for substitution. Depending on the relevance of Cys residues in the 3D structure such can be either substituted or not selected for substitution. Cys is often involved into formation of S—S bonds in proteins and is important for tertiary structure. Gly is a very flexible amino acid and substituting such with a larger amino acid such as Ala may also have a structural effect. Optionally, Pro residues can also be left out of the analysis as such are often involved in secondary structure formation.

More specifically the amino-acids within the accessible surface area are selected based on the calculated solvent-accessible surface area of side chains. Standard methods to calculate solvent accessibility can be applied. In a typical example a probe of 1.4 Å is used for calculations (a simplified version of H₂O molecule wherein such probe has a size similar to an H₂O molecule). In such calculations atoms of the amino-acid residues that touch the probe are classified as surface accessible atoms. Surface accessibility of each amino-acid is calculated in Å². Subsequently a ratio between the actual surface exposed area (in Å²) and theoretical probable surface exposure (in Å²) is calculated. Different cut-offs can be selected depending on the desired accuracy and the size of the protein. Such cut off can be selected from 0.5, 0.2, preferably such cut-off is between 0.05-0.1, more preferably such cut-off is 0.07. Such filtering step is useful to eliminate potentially misfolding proteins.

Further steps to reduce the amount of misfolded TREM1 proteins in the final array can be performed. For example, residues that cause breakage of more than one hydrogen bond between any of the original residues of each mutated patch (2 or 3 residues) and the rest of the protein are preferably avoided. Similarly any breakage in the salt-bridges should also be preferably avoided. Additionally, mutating residues that expose large hydrophobic areas of the protein is also avoided. In another embodiment, residues that cause breakage of more than two hydrogen bonds within the protein are also avoided. Similarly, any breakage in the salt-bridges should also be preferably avoided.

Hence, in a preferred embodiment of the method, the method excludes or filters out 1) patches that result in the breakage of hydrogen bonds (preferably maximum of 2 broken bonds allowed) and 2) salt bridges (preferably maximum 1 broken bond allowed), as well as 3) the exposure of large hydrophobic patches (preferably maximum 15 Å 2 of exposed hydrophobic surface allowed). The distance threshold to define a patch could be set between 6 and 6.5 Å and the minimal sidechain surface exposure could be set to 7%.

Optionally, further granularity can be achieved by performing a molecular dynamics simulation with any widely used simulations package (e.g. AMBER, GROMACS, DESMOND, etc.) with a subsequent analysis of interaction persistence. Hydrogen bonds and salt bridges that are present in a large fraction of the simulation trajectory can be considered “essential” and should not be broken by an Ala mutation, whereas bonds that are only observed in a small fraction of the simulation are likely to have little impact on the protein's stability.

Additionally, after all the patches of residues have been identified any redundancy in such is eliminated by eliminating the patches that generate redundancy. This step is optional as it could be beneficial to have some redundancy in the coverage of the accessible surface area, however having such redundancy might provide technical difficulty in generating mutant clones subsequently. Hence, such redundancy should be considered in the context of the protein size, complexity and technical limitations in designing the corresponding mutant proteins.

Ideally, the steps above are performed for the whole protein surface to make sure that maximum surface-accessible area is covered by the identified patches. It would be preferable to avoid having some parts of the surface-accessible area not covered by such patches. The purpose is to cover the solvent accessible surface while minimizing the number of generated misfolded proteins.

If, for example, using patches of 2 substitutions would not cover the whole surface-accessible area, additional patches consisting of 3 substitutions can be designed. Larger patches of more than 3 substitutions can also be used, however going beyond 3 substitutions may lead to misfolding of a mutant TREM1 protein. Hence, preferably patches containing 2 or 3 Ala substitutions are used. If desired additional single Ala substitution could also be selected. However, such may not provide the desired sensitivity compared to 2 or 3 substitutions.

The arrays of mutant TREM1 proteins having 2 or 3 Ala patches following this strategy are provided in the Examples.

The generated sequences of mutated TREM1 protein are subsequently produced for experimental testing. A typical way to produce such is by cloning the sequences into a suitable expression vector. As a control, the wild type sequence of the target protein of interest is also cloned.

An array of mutant TREM1 proteins can be produced using techniques known to the skilled person. Any suitable expression system for expressing proteins in target cells can be used. Preferably a mammalian cell system is used for expression of the cloned mutant peptides. Mammalian cells would allow for the mutant polypeptides to be secreted out of such cells and make testing such peptides easier. Any mammalian cell or cell line could be used as long as such allows for sufficient expression of each of the mutant peptides. In such a mammalian system a suitable expression vector can be used. Many mammalian expression vectors are commercially available. Typically such a vector will comprise a constitutive promoter, such as cytomegalovirus (CMV) promoter.

Each of the mutant TREM1 proteins could be fused to an Fc region, preferably human Fc domain. Use of Fc domain in such fusion proteins offers practical advantages, such as higher robustness in detection and ease of capturing such fusion proteins on a surface. Optionally one or more linker sequences can be introduced into the fusion protein sequence between the Fc domain and the target mutant protein if necessary, such as triple Ala linker.

Preferably, such fusion proteins comprising human Fc domain are expressed in mammalian Expi293 cells, or any other cells that can generate sufficient concentration of the protein.

Optionally, TREM1 proteins that might potentially misfold could be removed from the array by pre-screening the array using polyclonal antibodies (targeting multiple epitopes) against TREM1 or any commercial monoclonal antibodies of known epitopes which are suitable for ELISA assays (as such antibodies would recognize a structural epitope).

Finally, binding properties of an antibody to each of the mutant target proteins on the array are measured. Such measurements can be performed using any suitable method available. Preferably, such measurements are performed using a high-throughput method.

The affinity of a molecule of interest, as well as the extent to which such molecule inhibits binding to the target protein, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, mutant proteins are immobilized on a solid phase and exposed to ligands and/or the molecule of interest in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

Alternative platforms using techniques similar to SPR are provides by Cartera (carterra-bio.com) such as Carterra LSA Platform. It is a high throughput antibody characterization platform that combines flow printing microfluidics with high throughput surface plasmon resonance (SPR) detection technology.

Other types of platforms include techniques utilizing cell surface-expression arrays. An example of such platform is LigandTracer (ligandtracer.com) which is particularly suited to follow protein binding to cell-surface receptors and allows to measure on- and off-rates as well as affinities.

In order to simplify the measurements, each of the mutant proteins of the array could be fused to a molecule or a protein to allow to capture such on a surface for easier detection of binding properties.

Preferably the binding to each of the mutant proteins is determined using Bio-Layer Interferometry (BLI) is a label-free technology. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time (REF).

Typically arrays of 30, 60 cloned mutant proteins are used. However the size of such arrays depends on the size of the target protein and the desired coverage of the solvent-accessible area. Preferably the mutant proteins are provided on a 96 well plate or 384-well plate. Generally a BLI instrument can handle 96- or 384well plates for measurements.

When using BLI technology typically each sensor is exposed to a solution containing the molecule of interest (such as an antibody or a ligand) for which the binding site is being determined. The advantage of BLI technology is that is almost as sensitive as a normal BIACore, it is high throughput (96 clones can be tested at the same time) and uses disposable sensor tips so there is no need to regenerate the surface and reuse a chip as you would typically do with BIACore.

Different measurements of binding of a test antibody to the mutant TREM1 proteins can be used to determine which of the mutant proteins demonstrate reduced binding. Typically, dissociation constants or binding constants are measured. Typically, complete loss of binding or how quickly the molecule of interest is coming off the mutant protein can be measured. Appropriate controls are generally used when measuring the binding properties of the antibody. Commonly the binding properties are compared to parental sequence of the target protein (wild type, WT). Typically the majority of mutant proteins will show the same K_{d} as the WT. The mutant proteins showing a difference in binding should be considered. Typically, any dissociation constant difference of at least 2, 3, 4, 5, 6, 7, 8, 9, or 10 or more fold compared to wild-type TREM1 is considered. Preferably any difference of at least 3-fold is considered significant. The mutant TREM1 proteins that produce the results with low noise to signal resolutions are ignored or re-measured.

If desired mutant proteins comprising patches of different size, such as patches of 2 or 3 substitutions can be used on an array. Mutant proteins comprising single substitutions can also additionally be tested for binding properties if a higher precision is required, provided such offer sufficient sensitivity to obtain a measurable effect.

### Antibodies Binding to TREM1

The present invention provides anti-TREM1 antibodies that bind to human TREM1 (target polypeptide) and have functional and structural properties as described further herein.

The antibodies in the context of the present invention include whole antibodies and functionally active antibody fragments (i.e., molecules that contain an antigen binding domain that specifically binds an antigen, also termed antigen-binding fragments). Features described herein also apply to antibody fragments unless context dictates otherwise. The antibody may be (or derived from) polyclonal, monoclonal, multi-valent, multi-specific, bispecific, fully human, humanized or chimeric.

The antibodies described further in are specific antibody types and do not limit the scope of invention.

An antibody used according to the invention may be a monoclonal antibody or a polyclonal antibody, and is preferably a monoclonal antibody. An antibody used according to the invention may be a chimeric antibody, a CDR-grafted antibody (e.g., any appropriate acceptor variable region framework sequence may be used having regard to the class/type of the donor antibody from which the CDRs are derived, including mouse, primate and human framework regions), a nanobody, a human or humanized antibody. For the production of both monoclonal and polyclonal antibodies, the animal used to raise such antibodies is typically a non-human mammal such as a goat, rabbit, rat or mouse but the antibody may also be raised in other species.

Polyclonal antibodies may be produced by routine methods such as immunization of a suitable animal with an antigen of interest. Blood may be subsequently removed from such animal and the produced antibodies purified.

Monoclonal antibodies may be made by a variety of techniques, including but not limited to, the hybridoma method, recombinant DNA methods, phage-display methods, and methods utilizing transgenic animals containing all or a part of the human immunoglobulin loci. Some exemplary methods for making monoclonal antibodies are described herein.

For example, monoclonal antibodies may be prepared using the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp77-96, Alan R Liss, Inc., 1985).

Antibodies may also be generated using single lymphocyte antibody methods by cloning and expressing immunoglobulin variable region cDNAs generated from single lymphocytes selected for the production of specific antibodies by for example the methods described in WO9202551, WO2004051268 and WO2004106377.

Antibodies generated against the target polypeptide may be obtained, where immunization of an animal is necessary, by administering the polypeptide to an animal, preferably a non-human animal, using well-known and routine protocols, see for example Handbook of Experimental Immunology, D. M. Weir (ed.), Vol 4, Blackwell Scientific Publishers, Oxford, England, 1986). Many animals, such as rabbits, mice, rats, sheep, cows, camels or pigs may be immunized. However, mice, rabbits, pigs and rats are generally used.

Monoclonal antibodies can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280). In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US 2005/0079574, US2005/0119455, US2005/0266000, US2007/0117126, US2007/0160598, US2007/0237764, US2007/0292936, and US2009/0002360.

Screening for antibodies can be performed using assays to measure binding to the target polypeptide and/or assays to measure the ability of the antibody to block a particular interaction. An example of a binding assay is an ELISA, for example, using a fusion protein of the target polypeptide, which is immobilized on plates, and employing a conjugated secondary antibody to detect the antibody bound to the target. An example of a blocking assay is a flow cytometry based assay measuring the blocking of a ligand protein binding to the target polypeptide. A fluorescently labelled secondary antibody is used to detect the amount of such ligand protein binding to the target polypeptide.

Antibodies may be isolated by screening combinatorial libraries for antibodies with the desired activity or activities. For example, a variety of methods are known in the art for generating phage display libraries and screening such libraries for antibodies possessing the desired binding characteristics.

Antibodies or antibody fragments isolated from human antibody libraries are considered human antibodies or human antibody fragments.

The antibody may be a full-length antibody. More particularly the antibody may be of the IgG isotype. More particularly the antibody may be an IgG1 or IgG4.

The constant region domains of the antibody, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. It will also be known to the person skilled in the art that antibodies may undergo a variety of posttranslational modifications. The type and extent of these modifications often depends on the host cell line used to express the antibody as well as the cell culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

Alternatively, the antibody is an antigen-binding fragment.

For a review of certain antigen-binding fragments, see Hudson et al. Nat. Med. 9: 129-134 (2003). For a review of scFv fragments, see, e.g., Plückthun, in The Pharmacology of Monoclonal Antibodies, vol. 113, Rosenburg and Moore eds., (Springer Verlag, New York), pp. 269-315 (1994); see also WO 93/16185; and U.S. Pat. Nos. 5,571,894 and 5,587,458. Fab and F(ab′)₂ fragments comprising salvage receptor binding epitope residues and having increased in vivo half-life are disclosed in U.S. Pat. No. 5,869,046.

Antigen-binding fragments and methods of producing them are well known in the art, see for example Verma et al., 1998, Journal of Immunological Methods, 216, 165-181; Adair and Lawson, 2005. Therapeutic antibodies. Drug Design Reviews-Online 2(3):209-217. The Fab-Fv format was first disclosed in WO2009/040562 and the disulphide stabilized version thereof, the Fab-dsFv, was first disclosed in WO2010/035012, and TrYbe format is disclosed in WO2015/197772.

Various techniques have been developed for the production of antibody fragments. Such fragments might be derived via proteolytic digestion of intact antibodies (see, e.g., Morimoto et al., Journal of Biochemical and Biophysical Methods 24: 107-117 (1992) and Brennan et al, Science 229:81 (1985)). However, antibody fragments can also be produced directly by recombinant host cells. For example, antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)).

F(ab′)2 fragments can be isolated directly from recombinant host cell culture. The antibody may be a single chain Fv fragment (scFv). Such are described in WO 93/16185; U.S. Pat. Nos. 5,571,894; and 5,587,458. The antibody fragment may also be a “linear antibody,” e.g., as described in U.S. Pat. No. 5,641,870. Such linear antibody fragments may be monospecific or bispecific.

The antibody may be a Fab, Fab′, F(ab′)₂, Fv, dsFv, scFv, or dsscFv. The antibody may be a single domain antibody or a nanobody, for example VH or VL or VHH or VNAR. The antibody may be Fab or Fab′ fragment described in WO2011/117648, WO2005/003169, WO2005/003170 and WO2005/003171.

The antibody may be a disulfide-stabilized single chain variable fragment (dsscFv).

The disulfide bond between the variable domains VH and VL may be between two of the residues listed below:
V_{H}37+V_{L}95 see for example Protein Science 6, 781-788 Zhu et al(1997);
V_{H}44+V_{L}100 see for example Weatherill et al., Protein Engineering, Design & Selection, 25 (321-329), 2012;
V_{H}44+V_{L}105 see for example J Biochem. 118, 825-831 Luo et al(1995);
V_{H}45+V_{L}87 see for example Protein Science 6, 781-788 Zhu et al(1997);
V_{H}55+V_{L}101 see for example FEBS Letters 377 135-139 Young et al(1995);
V_{H}100+V_{L}50 see for example Biochemistry 29 1362-1367 Glockshuber et al(1990);
V_{H}100b+V_{L}49; see for example Biochemistry 29 1362-1367 Glockshuber et al(1990);
V_{H}98+V_{L} 46 see for example Protein Science 6, 781-788 Zhu et al(1997);
V_{H}101+V_{L}46; see for example Protein Science 6, 781-788 Zhu et al(1997);
V_{H}105+V_{L}43 see for example; Proc. Natl. Acad. Sci. USA Vol. 90 pp.7538-7542 Brinkmann et al(1993); or Proteins 19, 35-47 Jung et al(1994),
V_{H}106+V_{L}57 see for example FEBS Letters 377 135-139 Young et al(1995) and a position or positions corresponding thereto in a variable region pair located in the molecule.

The disulphide bond may be formed between positions VH44 and VL100.

It will be appreciated by the skilled person that antigen-binding fragments described herein may also be characterized as monoclonal, chimeric, humanized, fully human, multispecific, bispecific etc., and that discussion of these terms also relate to such fragments.

### Multi-Specific Antibodies

The antibodies of the present invention may be multi-specific antibodies.

Examples of multi-specific antibodies or antigen-binding fragments thereof, which also are contemplated for use in the context of the disclosure, include bi, tri or tetra-valent antibodies, Bis-scFv, diabodies, triabodies, tetrabodies, bibodies and tribodies (see for example Holliger and Hudson, 2005, Nature Biotech 23(9): 1126-1136; Schoonjans et al. 2001, Biomolecular Engineering, 17(6), 193-202).

A variety of multi-specific antibody formats have been generated. Different classifications have been proposed, but multispecific IgG antibody formats generally include bispecific IgG, appended IgG, multispecific (e.g. bispecific) antibody fragments, multispecific (e.g. bispecific) fusion proteins, and multispecific (e.g. bispecific) antibody conjugates, as described for example in Spiess et al., Alternative molecular formats and therapeutic applications for bispecific antibodies. Mol Immunol. 67(2015):95-106.

The antibody may be a bi-specific antibody. In one embodiment, the antibody comprises two antigen binding domains wherein one binding domain binds TREM1 and the other binding domain binds another antigen, i.e. each binding domain is monovalent for each antigen. In one embodiment, the antibody is a tetravalent bispecific antibody, i.e. the antibody comprises four antigen binding domains, wherein for example two binding domains bind TREM1 and the other two binding domains bind to another antigen. In one embodiment, the antibody is a trivalent bispecific antibody.

Techniques for making bispecific antibodies include, but are not limited to, CrossMab technology (Klein et al. Engineering therapeutic bispecific antibodies using CrossMab technology, Methods 154 (2019) 21-31), Knobs-in-holes engineering (e.g. WO1996027011, WO1998050431), DuoBody technology (e.g. WO2011131746), Azymetric technology (e.g. WO2012058768). Further technologies for making bispecific antibodies have been described for example in Godar et al., 2018, Therapeutic bispecific antibody formats: a patent applications review (1994-2017), Expert Opinion on Therapeutic Patents, 28:3, 251-276. Bispecific antibodies include in particular CrossMab antibodies, DAF (two-in-one), DAF (four-in-one), DutaMab, DT-lgG, Knobs-in-holes common LC, Knobs-in-holes assembly, Charge pair, Fab-arm exchange, SEEDbody, Triomab, LUZ-Y, Fcab, d,-body and orthogonal Fab.

The antibody construct may be a tri-specific antibody.

The antibody may be a multi-paratopic antibody.

In one embodiment, each binding domain is monovalent. Preferably each binding domain comprises no more than one VH and one VL.

Appended IgG classically comprise full-length IgG engineered by appending additional antigen-binding domain or antigen-binding fragment to the N- and/or C-terminus of the heavy and/or light chain of the IgG. Examples of such additional antigen-binding fragments include sdAb antibodies (e.g. VH or VL), Fv, scFv, dsscFv, Fab, scFab Appended IgG antibody formats include in particular DVD-IgG, IgG(H)-scFv, scFv-(H)lgG, IgG(L)-scFv, scFv-(L)IgG, lgG(L,H)-Fv, IgG(H)-V, V(H)—IgG, IgC(L)-V, V(L)-IgG, KIH IgG-scFab, 2scFv-IgG, lgG-2scFv, scFv4-Tg, Zybody and DVI-IgG (four-in-one), for example as described in Spiess et al., Alternative molecular formats and therapeutic applications for bispecific antibodies. Mol Immunol. 67(2015):95-106.

Multispecific antibody fragments include nanobody, nanobody-HSA, BiTEs, diabody, DART, TandAb, scDiabody, sc-Diabody-CH3, Diabody-CH3, Triple Body, Miniantibody; Minibody, Tri Bi minibody, scFv-CH3 KIH, Fab-scFv, scFv-CH-CL-scFv, F(ab′)₂, F(ab′)₂-scFv₂, scFv-KIH, Fab-scFv-Fc, Tetravalent HCAb, scDiabody-Fc, Diabody-Fc, Tandem scFv-Fc; and intrabody, as described, for example, Spiess et al., Alternative molecular formats and therapeutic applications for bispecific antibodies. Mol Immunol. 67(2015):95-106.

Multispecific fusion proteins include Dock and Lock, ImmTAC, HSAbody, scDiabody-HSA, and Tandem scFv-Toxin.

Multispecific antibody conjugates include IgG-IgG; Cov-X-Body; and scFv1-PEG-scFv₂.

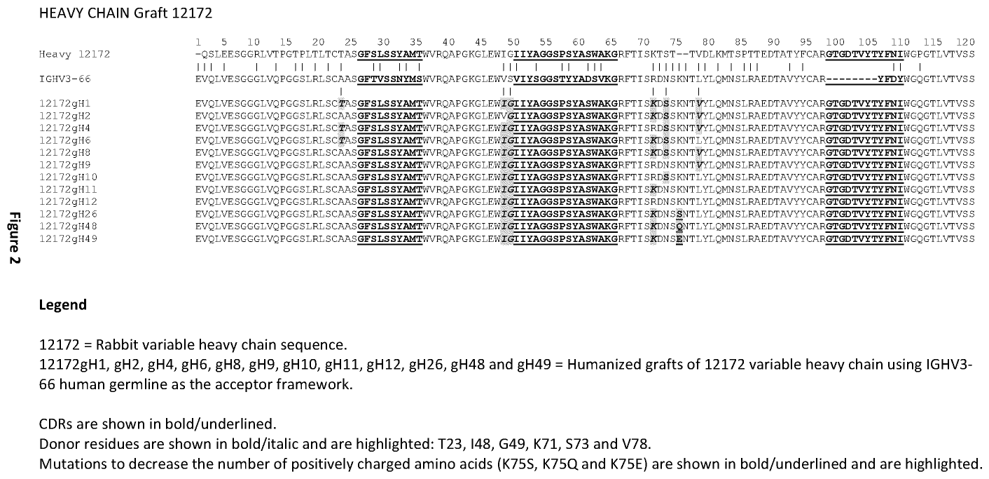

Additional multispecific antibody formats have been described for example in Brinkmann et al, The making of bispecific antibodies, mAbs, 9:2, 182-212 (2017), in particular in FIG. 2, for example tandem scFv, triplebody, Fab-VHH, taFv-Fc, scFv4-Ig, scFv2-Fcab, scFv4-IgG. Bibodies, tribodies and methods for producing such are disclosed, for example, in WO99/37791.

The antibody for use in the present invention may be a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in WO2015/197772. Another preferred antibody for use in the present invention fragment comprises a Fab linked to only one scFv or dsscFv, as described for example in WO2013/068571, and Dave et al., Mabs, 8(7) 1319-1335 (2016).

Another antibody for use in the present invention is a Knobs-into-holes antibody (“KiH”). It is a multi-specific antibody format consisting of heavy chain homodimers for heterodimerization (e.g., for the efficient production of bispecific antibodies, multi-specific antibodies, or one-armed antibodies). Generally, such technology involves introducing a protuberance (“knob”) at the interface of a first polypeptide (such as a first CH3 domain in a first antibody heavy chain) and a corresponding cavity (“hole”) in the interface of a second polypeptide (such as a second CH3 domain in a second antibody heavy chain), such that the protuberance can be positioned in the cavity so as to promote heterodimer formation and hinder homodimer formation. Protuberances are constructed by replacing small amino acid side chains from the interface of the first polypeptide (such as a first CH3 domain in a first antibody heavy chain) with larger side chains (e.g. arginine, phenylalanine, tyrosine or tryptophan).

Compensatory cavities of identical or similar size to the protuberances are created in the interface of the second polypeptide (such as a second CH3 domain in a second antibody heavy chain) by replacing large amino acid side chains with smaller ones (e.g. alanine, serine, valine, or threonine). The protuberance and cavity can be made by altering the nucleic acid encoding the polypeptides, e.g. by site-specific mutagenesis, or by peptide synthesis. Further details regarding “knobs-into-holes” technology is described in, e.g., U.S. Pat. Nos. 5,731,168; 7,695,936; WO 2009/089004; US 2009/0182127; Marvin md Z u, Acta Pharmacologica Sincia (2005) 26(6):649-658; Kontermann Acta Pharmacologica Sincia (2005) 26: 1-9; Ridgway et al, Prot Eng 9, 617-621 (1996); and Carter, J Immunol Meth 248, 7-15 (2001).

### Humanized, Human, and Chimeric Antibodies and Methods of Producing Such

The antibodies of the present invention may be, but are not limited to, humanized, fully human or chimeric antibodies.

In one embodiment the antibody is humanized. More particularly the antibody is a chimeric, human, or humanized antibody.

In certain embodiments, an antibody provided herein is a chimeric antibody. Examples of chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In one embodiment, the antibody is a humanized antibody.

Humanized antibodies may optionally further comprise one or more framework residues derived from the non-human species from which the CDRs were derived. It will be appreciated that it may only be necessary to transfer the specificity determining residues of the CDRs rather than the entire CDR (see for example, Kashmiri et al., 2005, Methods, 36, 25-34).

Suitably, the humanized antibody according to the present invention has a variable domain comprising human acceptor framework regions as well as one or more of the CDRs and optionally further including one or more donor framework residues.

Thus, provided in one embodiment is a humanized antibody wherein the variable domain comprises human acceptor framework regions and non-human donor CDRs.

When the CDRs or specificity determining residues are grafted, any appropriate acceptor variable region framework sequence may be used having regard to the class/type of the donor antibody from which the CDRs are derived, including mouse, primate and human framework regions.

Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: www.imgt.org. In embodiments, the acceptor framework is IGKV1-9 human germline, and/or IGHV3-66 human germline. In embodiments, the human framework contains 1-5, 1-4, 1-3 or 1-2 donor antibody amino acid residues.

In a humanized antibody of the present invention, the acceptor heavy and light chains do not necessarily need to be derived from the same antibody and may, if desired, comprise composite chains having framework regions derived from different chains.

In certain embodiments, an antibody provided herein is a human antibody. Human antibodies can be produced using various techniques known in the art.

Human antibodies comprise heavy or light chain variable regions or full length heavy or light chains that are “the product of” or “derived from” a particular germline sequence if the variable regions or full-length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody or fragment thereof that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acid sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Structural Features of the Antibodies

The antibody of the invention comprises a binding domain. A binding domain will generally comprise 6 CDRs, three from a heavy chain and three from a light chain. In one embodiment the CDRs are in a framework and together form a variable region. Thus, the antibody has a binding domain specific for antigen, said binding domain comprising a light chain variable region and a heavy chain variable region.

In one embodiment, the antibody comprises a heavy chain and a light chain wherein the heavy chain comprises a CH1 domain and the light chain comprises a CL domain, either kappa or lambda.

As demonstrated by the Examples of the present invention, different variants of variable regions of heavy and light chains had been produced and tested for their binding affinity. Those variants comprise same set of CDR sequences and demonstrate similar range of binding affinity. An overview of different structural elements of selected antibody variants is presented in Table 3.

TABLE 3
Amino-acid sequences of the anti-TREM1 antibodies

| | 12172 gL2gH11 | 12172 gL6gH6 |
|---|---|---|
| Feature | SEQ ID NO | SEQ ID NO |
| CDR-L1 | 11 | 11 |
| CDR-L2 | 12 | 12 |
| CDR-L3 | 13 | 13 |
| CDR-H1 | 14 | 14 |
| CDR-H2 | 15 | 15 |
| CDR-H3 | 16 | 16 |
| Light chain V region | 29 | 33 |
| Heavy chain V region | 79 | 57 |
| Light chain | 31 | 35 |
| Heavy chain IgG1 | 85 | 63 |
| Heavy chain IgG1 LALA | 87 | 65 |
| Heavy chain IgG4P | 81 | 59 |
| Heavy chain IgG4P FALA | 83 | 61 |

In one embodiment the present invention provides an antibody that binds to human TREM1, comprising a light chain variable domain which comprises at least one of:
a CDR-L1 comprising SEQ ID NO:11,
a CDR-L2 comprising SEQ ID NO: 12, and
a CDR-L3 comprising SEQ ID NO: 13.

In one embodiment the present invention provides an antibody that binds to human TREM1, comprising a light chain variable domain which comprises
a CDR-L1 comprising SEQ ID NO:11,
a CDR-L2 comprising SEQ ID NO: 12, and
a CDR-L3 comprising SEQ ID NO:13.

In one embodiment the present invention provides an antibody that binds to human TREM1, comprising a heavy chain variable domain which comprises at least one of:
a CDR-H1 comprising SEQ ID NO: 14,
a CDR-H2 comprising SEQ ID NO: 15, and
a CDR-H3 comprising SEQ ID NO: 16.

In one embodiment the present invention provides an antibody that binds to human TREM1, comprising a heavy chain variable domain which comprises
a CDR-H1 comprising SEQ ID NO:14,
a CDR-H2 comprising SEQ ID NO:15, and
a CDR-H3 comprising SEQ ID NO: 16.

The antibody molecules of the present invention may comprise a complementary light chain or a complementary heavy chain, respectively.

Hence, in one embodiment the present invention provides an antibody that binds to human TREM1, comprising:
a light chain variable region comprising:
   a CDR-L1 comprising SEQ ID NO:11,
   a CDR-L2 comprising SEQ ID NO: 12, and
   a CDR-L3 comprising SEQ ID NO: 13;
and a heavy chain variable region comprising:
   a CDR-H1 comprising SEQ ID NO: 14,
   a CDR-H2 comprising SEQ ID NO:15, and
   a CDR-H3 comprising SEQ ID NO: 16.

In one embodiment, an antibody of the present invention comprises a light chain variable region comprising the sequence given in SEQ ID NO:29 or SEQ ID NO:33.

In one embodiment, an antibody of the present invention comprises a heavy chain variable region comprising the sequence given in SEQ ID NO:57 or SEQ ID NO:79.

In one embodiment, an antibody of the present invention comprises alight chain variable region comprising the sequence given in SEQ ID NO:33 and a heavy chain variable region comprising the sequence given in SEQ ID NO:57.

In an alternative embodiment, an antibody of the present invention comprises a light chain variable region comprising the sequence given in SEQ ID NO:29 and a heavy chain variable region comprising the sequence given in SEQ ID NO:79.

In one embodiment, an antibody of the present invention is a full-length antibody comprising
a light chain variable region comprising:
   a CDR-L1 comprising SEQ ID NO:11,
   a CDR-L2 comprising SEQ ID NO: 12, and
   a CDR-L3 comprising SEQ ID NO: 13;
and a heavy chain variable region comprising:
   a CDR-H1 comprising SEQ ID NO: 14,
   a CDR-H2 comprising SEQ ID NO: 15, and
   a CDR-H3 comprising SEQ ID NO: 16.

In one embodiment, an antibody of the present invention is a IgG1 LALA comprising
a light chain variable region comprising:
   a CDR-L1 comprising SEQ ID NO:11,
   a CDR-L2 comprising SEQ ID NO: 12, and
   a CDR-L3 comprising SEQ ID NO: 13;
and a heavy chain variable region comprising:
   a CDR-H1 comprising SEQ ID NO: 14,
   a CDR-H2 comprising SEQ ID NO: 15, and
   a CDR-H3 comprising SEQ ID NO: 16.

In another embodiment, the antibody of the present invention is an IgG1 LALA comprising a light chain comprising the sequence given in SEQ ID NO: 35 and a heavy chain comprising the sequence given in SEQ ID NO: 65.

In another embodiment, the antibody of the present invention is an IgG1 LALA comprising a light chain comprising the sequence given in SEQ ID NO: 31 and a heavy chain comprising the sequence given in SEQ ID NO: 87.

In another embodiment an IgG4P is preferred. Several variants of the 12172 antibody described herein were tested in multiple assays to determine their physical-chemical properties, they all demonstrated very similar developability profiles with IgG4P variant having less preferable properties than the other variants. However, the IgG4P variant demonstrated surprising biological properties not observed with other variants and, hence, is a preferred variant for applications where such properties are beneficial. For example, in the treatment of a condition where such properties provide a therapeutic effect.

IgG4P contains the Ser-228-Pro mutation in the hinge region where numbering is according to EU numbering (Ser-241-Pro according to Kabat numbering) to improve hinge stability (Angal S et al, (1993), Mol Immunol, 30(1), 105-108).

Hence, in one embodiment, an antibody of the present invention is an IgG4P comprising
a light chain variable region comprising:
   a CDR-L1 comprising SEQ ID NO:11,
   a CDR-L2 comprising SEQ ID NO: 12, and
   a CDR-L3 comprising SEQ ID NO: 13;
and a heavy chain variable region comprising:
   a CDR-H1 comprising SEQ ID NO: 14,
   a CDR-H2 comprising SEQ ID NO: 15, and
   a CDR-H3 comprising SEQ ID NO: 16.

In yet another embodiment, the antibody of the present invention is an IgG4P comprising a light chain comprising the sequence given in SEQ ID NO: 35 and a heavy chain comprising the sequence given in SEQ ID NO: 59.

In a more specific embodiment, the antibody of the present invention is an IgG4P comprising a light chain comprising the sequence given in SEQ ID NO: 31 and a heavy chain comprising the sequence given in SEQ ID NO: 81.

### Functional Properties of the Anti-TREM1 Antibodies

In one embodiment, the antibody of the present invention is a neutralizing antibody. Preferably the antibody according to the present invention is neutralizing one or more TREM1 activities.

The antibodies of the present invention specifically bind human TREM1, and more specifically, a particular region within the extracellular domain of human TREM1. In some embodiments, the antibodies specifically bind to a different or minimally overlapping site on TREM1 to which a TREM1 ligand (e.g., PGLYRP1) binds. In some embodiments, the antibodies are antagonist antibodies, i.e., they inhibit or suppress the activity of TREM1 on cells. Such cells might be monocytes, macrophages, and/or neutrophils. In some embodiments, the antibodies may specifically bind to TREM1 allosterically, rather than orthosterically to a single ligand, and, hence, provide more effective inhibition of binding of other ligands which bind at a different site on TREM1 than PGLYRP1.

As demonstrated by the Examples, PGLYRP1 binds to an epitope on TREM1, said epitope comprising residues selected from the list consisting of E27, D42-E46, A49, Y90-L95, and F126 of human TREM1 (SEQ ID NO: 1) as determined at less than 4 Å contact distance.

In one particular embodiment, the present invention provides an antibody that binds to a region on TREM1 that is different from the binding site of PGLYRP1 such that the binding still prevents the interaction between TREM1 and PGLYRP1.

In some embodiments, the anti-TREM1 antibodies show very weak binding to cynomolgus TREM1. In some embodiments, the anti-TREM1 antibodies show no detectable binding to mouse, rat, pig or dog TREM1.

In some embodiments, the anti-TREM1 antibodies decrease the release of multiple cytokines and chemokines, such as, CCL-3, CCL-20, CXCL-9, GM-CSF, IFN-γ, IL-1α, IL-1β, IL-6, IL-10, IL-12p40, IL-15, IL-18, IL-27, TNF-α, and TNF-β from activated human monocytes.

In some embodiments, the anti-TREM1 antibody is an IgG4P and significantly increases the release of IL-1R antagonist (IL-1RA), an anti-inflammatory negative regulator of the IL-1 pathway, from primary human monocytes.

An antibody according to the present invention is specific for human TREM1.

In some embodiments, the antibody binds to human TREM1 with sufficient affinity and specificity. In certain embodiments, the antibody binds human TREM1 with a KD of about any one of 1 μM, 100 nM, 50 nM, 40 nM, 30 nM, 20 nM, 10 nM, 5 nM, 1 nM, 0.5 nM, including any range in between these values. In one embodiment, the antibody according to the present invention binds human TREM1 with a KD of less than 600 μM. In more specific embodiment, the antibody according to the present invention binds human TREM1 with a KD of 300-1200 μM, more preferably between 300-600 μM.

The affinity of an antibody, as well as the extent to which an antibody inhibits binding, can be determined by the skilled person using conventional techniques, for example those described by Scatchard et al. (Ann. KY. Acad. Sci. 51:660-672 (1949) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

Preferably the antibody according to the present invention is specific for human TREM1.

Disclosure herein relating to antibodies, particularly with respect to binding affinity and specificity, and activity, also is applicable to antigen-binding fragments and antibody-like molecules.

### Antibodies Binding to the Same Epitope

Antibodies may compete for binding to TREM1 with, or bind to the same epitope as, those defined above in terms of light-chain, heavy-chain, light chain variable region (LCVR), heavy chain variable region (HCVR) or CDR sequences.

In particular, the present invention provides an antibody that competes for binding to TREM1 with, or bind to the same epitope as, an antibody which comprises a CDR-L1/CDR-L2/CDR-L3/CDR-H1/CDR-H2/CDR-H3 sequence combination of SEQ ID NOs: 11/12/13/14/15/16. An antibody may compete for binding to TREM1 with, or bind to the same epitope as, an antibody which comprises a LCVR and HCVR sequence pair of SEQ ID NOs: 29/79. An antibody may compete for binding to TREM1 with or bind to the same epitope as an IgG4P comprising a CDR-L1/CDR-L2/CDR-L3/CDR-H1/CDR-H2/CDR-H3 sequence combination of SEQ ID NOs: 11/12/13/14/15/16.

In some embodiments, the anti-TREM1 antibody binds to an epitope on human TREM1, said epitope comprising residues E26, E27, K28, Y29, E30, L31, K32 and Q35 (where the numbering is according to SEQ ID NO: 1). Such epitope can be determined using the method disclosed herein, which involved designing an array of mutant TREM1 proteins and measuring the binding of said antibody to the mutant TREM1 proteins comprising 2 or 3 of said residues being mutated into a smaller amino acid, such as Ala.

In one embodiment, the present invention provides an IgG4P antibody that binds to an epitope of human TREM1, the epitope comprising residues E26, E27, K28, Y29, E30, L31, K32 and Q35 of human TREM1 (SEQ ID NO: 1).

In one embodiment, the present invention provides an anti-TREM1 antibody which binds to an epitope on TREM1, said epitope comprising at least 3, at least 4, at least 5, at least 6, at least 7, at least 8, at least 9, at least 10, or all of residues selected from the list consisting of E26, E27, K28, Y29, E30, L31, K32, Q35, T36, D38, K40, D42, R97, D127, T134 and G136 of human TREM1 (SEQ ID NO: 1) as determined at less than 4 Å contact distance.

In one embodiment, the present invention provides an IgG4P antibody that binds to an epitope of human TREM1, said epitope comprising at least 3, at least 4, at least 5, at least 6, at least 7, at least 8, at least 9, at least 10, or all of residues selected from the list consisting of E26, E27, K28, Y29, E30, L31, K32, Q35, T36, D38, K40, D42, R97, D127, T134 and G136 of human TREM1 (SEQ ID NO: 1) as determined at less than 4 Å contact distance.

In some embodiments, the present invention provides an anti-TREM1 antibody which binds to a different epitope than PGLYRP1. In some embodiments, the present invention provides an anti-TREM1 antibody which binds to an epitope on TREM1, said epitope comprising not more that 1 or 2 residues selected from the list consisting of E27, D42-E46, A49, Y90-L95, and F126 of human TREM1 (SEQ ID NO: 1) as determined at less than 4 Å contact distance.

The epitope can be identified by any suitable binding site mapping method known in the art in combination with any one of the antibodies provided by the present invention. A specific method is provided by the present disclosure that is relying on arrays of mutant TREM1 proteins to establish which of the mutant residues are important for binding for a particular antibody. Using such method it is possible to identify antibodies that bind to essentially the same amino acid residues as the antibodies provided by the present invention. Other examples of epitope mapping methods include screening peptides of varying lengths derived from full length target protein for binding to the antibody or fragment thereof of the present invention and identify a fragment that can specifically bind to the antibody containing the sequence of the epitope recognized by the antibody. Target peptides may be produced synthetically. Peptides that bind the antibody can be identified by, for example, mass spectrometric analysis. In another example, NMR spectroscopy or X-ray crystallography can be used to identify the epitope bound by an antibody of the present invention. Typically, when the epitope determination is performed by X-ray crystallography, amino acid residues of the antigen within 4 Å from CDRs are considered to be amino acid residues part of the epitope. Once identified, the epitope may serve for preparing fragments which bind an antibody of the present invention and, if required, used as an immunogen to obtain additional antibodies which bind the same epitope.

In one embodiment the epitope of the antibody is determined by X-ray crystallography.

One can easily determine whether an antibody binds to the same epitope as, or competes for binding with, a reference antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention or the reference antibody causes a conformation change in the antigen and hence preventing the binding of the test antibody.

To determine if an antibody competes for binding with a reference antibody, the above-described binding methodology is performed in two different experimental setups. In a first setup, the reference antibody is allowed to bind to the antigen under saturating conditions followed by assessment of binding of the test antibody to the antigen. In a second setup, the test antibody is allowed to bind to the antigen under saturating conditions followed by assessment of binding of the reference antibody to the protein/peptide. If, in both experimental setups, only the first (saturating) antibody is capable of binding to the protein/peptide, then it is concluded that the test antibody and the reference antibody compete for binding to the antigen. As will be appreciated by the skilled person, an antibody that competes for binding with a reference antibody may not necessarily bind to the identical epitope as the reference antibody, but may sterically block binding of the reference antibody by binding an overlapping or adjacent epitope or cause a conformational change leading to the lack of binding.

Two antibodies bind to the same or overlapping epitope if each competitively inhibits (blocks) binding of the other to the antigen. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same part of the antigen as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Antibody Variants

In certain embodiments, antibody variants having one or more amino acid substitutions, insertions, and/or deletions are provided. Sites of interest for substitutional mutagenesis include the CDRs and FRs. Amino acid substitutions may be introduced into an antibody of interest and the products screened for a desired activity, e.g., retained/improved antigen binding, decreased immunogenicity, or improved ADCC or CDC.

In certain embodiments, amino acid sequence variants of the antibodies described herein are contemplated. For example, it may be desirable to improve the binding affinity and/or other biological properties of the antibody. Amino acid sequence variants of the anti-TREM1 antibody may be prepared by introducing appropriate modifications into the nucleotide sequence encoding the protein, or by peptide synthesis. Such modifications include, for example, deletions from, and/or insertions into and/or substitutions of residues within the amino acid sequences (such as in one or more CDRs and/or framework sequences or in a VH and/or a VL domain) of the anti-TREM1 antibody. Any combination of deletion, insertion, and substitution can be made to arrive at the final construct, provided that the final construct possesses the desired characteristics.

In certain embodiments of the variant VH and VL sequences provided herein, each HVR either is unaltered, or contains no more than one, two or three amino acid substitutions.

It will be appreciated that one or more amino acid substitutions, additions and/or deletions may be made to the CDRs provided by the present invention without significantly altering the ability of the antibody to bind to TREM1 and to neutralize TREM1 activity. The effect of any amino acid substitutions, additions and/or deletions can be readily tested by one skilled in the art, for example by using the methods described herein, particularly those illustrated in the Examples, to determine TREM1 binding and inhibition of the TREM1 interactions with its natural ligands.

Consequently, in certain embodiments of the variant VH and VL sequences, each CDR either contains no more than one, two or three amino acid substitutions, wherein such amino-acid substitutions are conservative, and wherein the antibody retains its binding properties to TREM1.

Accordingly, the present invention provides an anti-TREM1 antibody comprising one or more CDRs selected from CDR-L1 (comprising SEQ ID NO: 11), CDR-L2 (comprising SEQ ID NO: 12), CDR-L3 (comprising SEQ ID NO: 13), CDR-H1 (comprising SEQ ID NO:14), CDR-H2 (comprising SEQ ID NO: 15) and CDR-H3 (comprising SEQ ID NO: 16) in which one or more amino acids in one or more of the CDRs has been substituted with another amino acid, for example a similar amino acid as defined herein below.

In one embodiment, the present invention provides an anti-TREM1 antibody comprising CDR-L1 (comprising SEQ ID NO:11), CDR-L2 (comprising SEQ ID NO:12), CDR-L3 (comprising SEQ ID NO: 13), CDR-H1 (comprising SEQ ID NO: 14), CDR-H2 (comprising SEQ ID NO: 15) and CDR-H3 (comprising SEQ ID NO:16), for example in which one or more amino acids in one or more of the CDRs has been substituted with another amino acid, such as a similar amino acid as defined herein below.

In one embodiment, the present invention provides an anti-TREM1 antibody CDR-L2 (comprising SEQ ID NO:12) wherein the first amino acid of SEQ ID NO: 12 has been substituted by another amino acid. More particularly the K is substituted by S.

In one embodiment, an anti-TREM1 antibody of the present invention comprises a light chain variable domain which comprises three CDRs wherein the sequence of CDR-L1 comprises a sequence that has at least 70%, 80%, 90%, 91%, 92%, 93%, 94%, 95%, 96%, 97%, 98%, or 99% identity or similarity to the sequence given in SEQ ID NO:11, CDR-L2 comprises a sequence that has at least 70%, 80%, 90%, 95% or 98% identity or similarity to the sequence given in SEQ ID NO:12 and/or CDR-L3 comprises a sequence that has at least 70%, 80%, 90%, 91%, 92%, 93%, 94%, 95%, 96%, 97%, 98%, or 99% identity or similarity to the sequence given in SEQ ID NO: 13.

In one embodiment, an anti-TREM1 antibody of the present invention comprises a heavy chain variable domain which comprises three CDRs wherein the sequence of CDR-H1 comprises a sequence that has at least 70%, 80%, 90%, 91%, 92%, 93%, 94%, 95%, 96%, 97%, 98%, or 99% identity or similarity to the sequence given in SEQ ID NO: 14, CDR-H2 comprises a sequence that has at least 70%, 80%, 90%, 91%, 92%, 93%, 94%, 95%, 96%, 97%, 98%, or 99% identity or similarity to the sequence given in SEQ ID NO: 15 and/or CDR-H3 comprises a sequence that has at least 70%, 80%, 90%, 91%, 92%, 93%, 94%, 95%, 96%, 97%, 98%, or 99% identity or similarity to the sequence given in SEQ ID NO: 16.

In one embodiment, an anti-TREM1 antibody of the present invention comprises alight chain variable region comprising a sequence having at least 70%, 80%, 90%, 91%, 92%, 93%, 94%, 95%, 96%, 97%, 98%, or 99% identity or similarity to the sequence given in SEQ ID NO:29.

In one embodiment, an antibody of the present invention comprises a heavy chain variable region comprising a sequence having at least 70%, 80%, 90%, 91%, 92%, 93%, 94%, 95%, 96%, 97%, 98%, or 99% identity or similarity to the sequence given in SEQ ID NO:79.

In one embodiment, an anti-TREM1 antibody of the present invention comprises alight chain variable region and a heavy chain variable region, wherein the light chain variable region comprises a sequence having at least 70%, 80%, 90%, 91%, 92%, 93%, 94%, 95%, 96%, 97%, 98%, or 99% identity or similarity to given in SEQ ID NO:29 and/or the heavy chain variable region comprises a sequence having at least 70%, 80%, 90%, 91%, 92%, 93%, 94%, 95%, 96%, 97%, 98%, or 99% identity or similarity to given in SEQ ID NO:79.

In one embodiment, an anti-TREM1 antibody of the present invention comprises CDR-L1/CDR-L2/CDR-L3/CDR-H1/CDR-H2/CDR-H3 sequences comprising SEQ ID NOs:11/12/13/14/15/16 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO:29 and 79 respectively.

In one embodiment the anti-TREM1 antibody of the present invention is a IgG4P comprising a light chain comprising sequence having at least 70%, 80%, 90%, 91%, 92%, 93%, 94%, 95%, 96%, 97%, 98%, or 99% identity or similarity to the sequence given in SEQ ID NO:31 and a heavy chain comprising sequence having at least 70%, 80%, 90%, 91%, 92%, 93%, 94%, 95%, 96%, 97%, 98%, or 99% identity or similarity to the sequence given in SEQ ID NO:81.

In one embodiment, an anti-TREM1 antibody of the present invention is a IgG4P comprising CDR-L1/CDR-L2/CDR-L3/CDR-H1/CDR-H2/CDR-H3 sequences given in SEQ ID NOs:11/12/13/14/15/16 respectively, and the remainder of the of the light chain and heavy chain has at least 70%, 80%, 90%, 91%, 92%, 93%, 94%, 95%, 96%, 97%, 98%, or 99% identity or similarity to SEQ ID Nos: 31 and 81 respectively.

In one embodiment, an antibody of the present invention comprises a light chain variable region and a heavy chain variable region, wherein the light chain variable region comprises the sequence given in SEQ ID NO:29, wherein one or more residues at the positions 1, 2, 3, 18 and 50 have been substituted by another amino-acid; and the heavy chain variable region comprises the sequence given in SEQ ID NO:79, wherein one or more residues at the positions 23, 48, 49, 71, 73, 75 and 78 have been substituted by another amino-acid.

Sequence Identity and similarity

Degrees of identity and similarity between sequences can be readily calculated. The “% sequence identity” (or “% sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) amino-acids (e.g., identical amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to obtain the % sequence identity or percent sequence similarity.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). Polypeptide sequences also can be compared using FASTA using default or recommended parameters. FASTA (e.g., FASTA2 and FASTA3) provides alignments and percent sequence identity of the regions of the best overlap between the query and search sequences.

In certain embodiments, substitutions, insertions, or deletions may occur within one or more CDR so long as such alterations do not substantially reduce the ability of the antibody to bind the target.

For example, conservative alterations that do not substantially reduce binding affinity may be made in CDRs. Such alterations may be made outside of antigen contacting residues in the CDRs.

Conservative substitutions are shown in Table 4 together with more substantial “exemplary substitutions”.

TABLE 4
Examples of amino-acid substitutions

| Original Residue | Exemplary Substitutions | Conservative Substitution |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys(C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |

Substantial modifications in the biological properties of an antibody variant can be accomplished by selecting substitutions that differ significantly in their effect on maintaining the structure of the polypeptide backbone in the area of the substitution, the charge or hydrophobicity of the molecule at the target site, or the bulk of the side chain. Amino acids may be grouped according to similarities in the properties of their side chains (in A. L. Lehninger, Biochemistry second ed., pp. 73-75, Worth Publishers, New York (1975))

One type of substitutional variant involves substituting one or more CDR region residues of a parent antibody (humanized or human antibody). Generally, the resulting variant(s) selected for further study will have changes in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or residues that contact antigen, with the resulting variant VH or VL being tested for binding affinity. Affinity maturation by constructing and reselecting from secondary libraries has been described, e.g., in Hoogenboom et al. Methods in Molecular Biology 178: 1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity.

One of the methods that can be used for identification of residues or regions of an antibody that may be targeted for mutagenesis is alanine scanning mutagenesis (Cunningham and Wells (1989) Science, 244: 1081-1085). In this method, a residue or a number of target residues are identified and replaced by alanine to determine whether the interaction of the antibody with antigen is affected. Alternatively, or additionally, an X-ray structure of an antigen-antibody complex can be used to identify contact points between the antibody and its antigen. Variants may be screened to determine whether they contain the desired properties.

### Constant Region Variants

In some embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

Antibodies with increased half-lives and improved binding to the neonatal Fc receptor (FcRn) are described in US2005/0014934A1. Those antibodies comprise an Fc region with one or more substitutions therein which improve binding of the Fc region to FcRn.

In certain embodiments, an antibody variant comprises an Fc region with one or more amino acid substitutions which improve ADCC, e.g., substitutions at positions 298, 333, and/or 334 of the Fc region (EU numbering of residues).

Antibodies with reduced effector function include those with substitution of one or more of Fc region residues 234, 235, 237, 238, 265, 269, 270, 297, 327 and 329 (see, e.g., U.S. Pat. No. 6,737,056). Such Fc mutants include Fc mutants with substitutions at two or more of amino acid positions 265, 269, 270, 297 and 327 wherein the amino acid residue is numbered according to the EU numbering system.

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcRI, FcγRII and FcγRIII. FcR expression on hematopoietic cells is summarized in Ravetch and Kinet, Annu. Rev. Immunol. 9:457-492 (1991). Non-limiting examples of in vitro assays to assess ADCC activity of a molecule of interest is described in U.S. Pat. Nos. 5,500,362; 5,821,337. Alternatively, or additionally, ADCC activity of the molecule of interest may be assessed in vivo, e.g., in an animal model such as that disclosed in Clynes et. al. Proc. Nat 1 Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al, J. Immunol. Methods 202: 163 (1996); Cragg, M. S. et al, Blood 101:1045-1052 (2003); and Cragg, M. S. and M. I Glennie, Blood 103:2738-2743 (2004)). FcRn binding and in vivo clearance/half-life determinations can also be performed using methods known in the art (see, e.g., Petkova, S. B. et al, Int l. Immunol. 18(12): 1759-1769 (2006)).

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used.

In some embodiments, the antibody is an IgG1 LALA, a mutant of the wild-type human IgG1 isoform in which amino acid substitutions L234A/L235 Å (according to EU numbering) in the constant region of IgG1 have been introduced.

In some embodiments, the antibody is an IgG4P, a mutant of the wild-type human IgG4 isoform in which amino acid 228 (according to EU numbering) is replaced by proline, as described for example in Angal et al., Molecular Immunology, 1993, 30 (1), 105-108.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

### Humanized, Human, and Chimeric Antibodies

The antibodies of the present invention may be, but are not limited to, humanized, fully human or chimeric antibodies.

In one embodiment, the antibody is humanized. More particular the anti-TREM1 antibody is a chimeric, human, or humanized antibody.

In certain embodiments, an antibody provided herein is a chimeric antibody. Examples of chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In another example, a chimeric antibody is a “class switched” antibody in which the class or subclass has been changed from that of the parent antibody.

Chimeric antibodies are composed of elements derived from two different species such that the element retains the characteristics of the species from which it is derived. Generally a chimeric antibody will comprise a variable region from one species, for example a mouse, rat, rabbit or similar and constant region from another species such as a human.

In certain embodiments, a chimeric antibody is a humanized antibody.

It will be appreciated that it may only be necessary to transfer the specificity determining residues of the CDRs rather than the entire CDR (see for example, Kashmiri et al., 2005, Methods, 36, 25-34). Humanized antibodies may optionally further comprise one or more framework residues derived from the non-human species from which the CDRs were derived.

Suitably, the humanized antibody according to the present invention has a variable domain comprising human acceptor framework regions as well as one or more of the CDRs and optionally further including one or more donor framework residues.

In one embodiment the antibody is a humanized antibody, wherein the variable domain comprises human acceptor framework regions and non-human donor CDRs.

When the CDRs are grafted, any appropriate acceptor variable region framework sequence may be used having regard to the class/type of the donor antibody from which the CDRs are derived, including mouse, primate and human framework regions.

Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: www.imgt.org. In embodiments, the acceptor framework is IGKV1-9 human germline and/or IGHV3-66 human germline. In embodiments, the human framework contains 1-5, 1-4, 1-3 or 1-2 donor antibody amino acid residues.

In a humanized antibody of the present invention, the acceptor heavy and light chains do not necessarily need to be derived from the same antibody and may, if desired, comprise composite chains having framework regions derived from different chains.

In some embodiments, the antibody is a human antibody. Human antibodies can be produced using various techniques known in the art. More particular the anti-TREM1 antibody comprises a human antibody heavy chain constant region and a human light chain constant region.

Human antibodies comprise heavy or light chain variable regions or full length heavy or light chains that are derived from a particular germline sequence if the variable regions or full-length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is derived from a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is derived from a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acid sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Human antibodies may be produced by a number of methods known to those of skill in the art. Human antibodies can be made by the hybridoma method using human myeloma or mouse human heteromyeloma cells lines (Kozbor, J Immunol; (1984) 133:3001; Brodeur, Monoclonal Isolated Antibody Production Techniques and Applications, pp51-63, Marcel Dekker Inc, 1987). Alternative methods include the use of phage libraries or transgenic mice both of which utilize human variable region repertories (Winter G; (1994) Annu Rev Immunol 12:433-455, Green LL, (1999) J Immunol Methods 231:1 1-23). Human antibodies may be produced, for example, by mice in which the murine immunoglobulin variable and optionally the constant region genes have been replaced by their human counterparts as described, for example, in U.S. Pat. Nos. 5,545,806, 5,569,825, 5,625,126, 5,633,425, 5,661,016, and 5,770,429.

### Effector Molecules

If desired an antibody according to the present invention may be conjugated to one or more effector molecule(s). In one embodiment the antibody is not attached an effector molecule.

It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thiotepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as 11I n and 90Y, Lu177, Bismuth213, Californium252, Iridiuml92 and Tungsten188/Rhenium188; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO2005/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment, the polymer is albumin or a fragment thereof, such as human serum albumin or a fragment thereof.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumor, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example, the antibody according to the present invention are attached to poly(ethyleneglycol) (PEG) moieties. In one particular embodiment, the antigen-binding fragment according to the present invention and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond.

Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

In one embodiment, the antibody is a modified Fab′ fragment having at the C-terminal end of its heavy chain a modified hinge region containing at least one cysteine residue to which an effector molecule is attached. Suitably the effector molecule is PEG and is attached using the methods described in (WO 98/25971 and WO 2004072116 or in WO 2007/003898. Effector molecules may be attached to antibody fragments using the methods described in International patent applications WO 2005/003169, WO 2005/003170 and WO 2005/003171.

In one embodiment the antibody is not attached an effector molecule.

### Polynucleotides and Vectors

The present invention also provides an isolated polynucleotide encoding the antibody or a part thereof according to the present invention (such as Amino-acid SEQ IDs listed in Table 5). The isolated polynucleotide according to the present invention may comprise synthetic DNA, for instance produced by chemical processing, cDNA, genomic DNA or any combination thereof.

TABLE 5
Amino-acid sequences of the anti-TREM1 antibodies and their
corresponding nucleic acid sequences.

| | Amino-acid | Nucleic acid |
|---|---|---|
| Antibody sequence | SEQ ID NO | SEQ ID NO |
| 12172 gL6gH6 light chain V region | 33 | 34 |
| 12172 gL6gH6 heavy chain V region | 57 | 58 |
| 12172 gL6gH6 light chain | 35 | 36 |
| 12172 gL6gH6 heavy chain IgG1 | 63 | 64 |
| 12172 gL6gH6 heavy chain IgG1 LALA | 65 | 66 |
| 12172 gL6gH6 heavy chain IgG4P | 59 | 60 |
| 12172 gL6gH6 heavy chain IgG4P FALA | 61 | 62 |
| 12172 gL2gH11 light chain V region | 29 | 30 |
| 12172 gL2gH11 heavy chain V region | 79 | 80 |
| 12172 gL2gH11 light chain | 31 | 32 |
| 12172 gL2gH11 heavy chain IgG1 | 85 | 86 |
| 12172 gL2gH11 heavy chain IgG1 LALA | 87 | 88 |
| 12172 gL2gH11 heavy chain IgG4P | 81 | 82 |
| 12172 gL2gH11 heavy chain IgG4P FALA | 83 | 84 |

Examples of suitable sequences are provided herein. Thus in one embodiment the present invention provides an isolated polynucleotide encoding an antibody, comprising a sequence given in SEQ ID NOs 34, 58, 36, 64, 66, 60, 62, 30, 80, 32, 86, 88, 82, or 84.

In one embodiment, the present invention provides an isolated polynucleotide encoding the heavy chain of an IgG1 LALA or IgG4P antibody of the present invention which comprises the sequence given in SEQ ID NO: 88 or 82 respectively.

Also provided is an isolated polynucleotide encoding the light chain of an IgG1 LALA or IgG4P antibody of the present invention which comprises the sequence given in SEQ ID NO: 32.

In another embodiment, the present invention provides an isolated polynucleotide encoding the heavy chain and the light chain of an IgG4P antibody of the present invention in which the polynucleotide encoding the heavy chain comprises the sequence given in SEQ ID NO: 82 and the polynucleotide encoding the light chain comprises the sequence given in SEQ ID NO: 32.

The present invention also provides for a cloning or expression vector comprising one or more polynucleotides described herein. In one example, the cloning or expression vector according to the present invention comprises one or more isolated polynucleotides comprising a sequence selected from SEQ ID NO: 34, 58, 36, 64, 66, 60, 62, 30, 80, 32, 86, 88, 82, or 84.

Standard techniques of molecular biology may be used to prepare DNA sequences coding for the antibody or antigen-binding fragment thereof of the present invention. Desired DNA sequences may be synthesized completely or in part using oligonucleotide synthesis techniques. Site-directed mutagenesis and polymerase chain reaction (PCR) techniques may be used as appropriate.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

### Host Cells for Production of the Antibodies and Antigen-Binding Fragments Thereof

Also provided is a host cell comprising one or more isolated polynucleotide sequences according to the invention or one or more cloning or expression vectors comprising one or more isolated polynucleotide sequences encoding an antibody of the present invention. Any suitable host cell/vector system may be used for expression of the polynucleotide sequences encoding the antibody or antigen-binding fragment thereof of the present invention. Bacterial, for example E. coli, and other microbial systems may be used or eukaryotic, for example mammalian, host cell expression systems may also be used. Suitable mammalian host cells include CHO, myeloma or hybridoma cells.

In a further embodiment, a host cell comprising such nucleic acid(s) or vector(s) is provided. In one such embodiment, a host cell comprises (e.g., has been transformed with): (1) a vector comprising a nucleic acid that encodes an amino acid sequence comprising the VL of the anti-TREM1 antibody and an amino acid sequence comprising the VH of the anti-TREM1 antibody, or (2) a first vector comprising a nucleic acid that encodes an amino acid sequence comprising the VL of the anti-TREM1 antibody and a second vector comprising a nucleic acid that encodes an amino acid sequence comprising the VH of the anti-TREM1 antibody. In one embodiment, the host cell is eukaryotic, e.g. a Chinese Hamster Ovary (CHO) cell or lymphoid cell (e.g., Y0, NS0, Sp20 cell). In one embodiment, the host cell is prokaryotic, e.g. an E. coli cell. In one embodiment, a method of making an anti-TREM1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable types of Chinese Hamster Ovary (CHO cells) for use in the present invention may include CHO and CHO-K1 cells including dhfr CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NS0 myeloma cells and SP2 cells, COS cells. The host cell may be stably transformed or transfected with the isolated polynucleotide sequences or the expression vectors according to the present invention.

### Process for the Production of the Antibodies

The present invention also provides a process for the production of an antibody according to the present invention comprising culturing a host cell according to the present invention under conditions suitable for producing the antibody according to the invention and isolating the antibody.

The antibody may comprise only a heavy or light chain polypeptide, in which case only a heavy chain or light chain polypeptide coding sequence needs to be used to transfect the host cells. For production of antibodies or antigen-binding fragments thereof comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Thus, there is provided a process for culturing a host cell and expressing an antibody, isolating the antibody and optionally purifying the antibody to provide an isolated antibody. In one embodiment, the process further comprises the step of conjugating an effector molecule to the isolated antibody.

The present invention also provides a process for the production of an antibody according to the present invention comprising culturing a host cell containing a vector of the present invention under conditions suitable for leading to expression of protein from DNA encoding the antibody molecule of the present invention and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

The antibodies according to the present invention are expressed at good levels from host cells. Thus the properties of the antibodies appear to be optimized for commercial processing.

### Purified Antibody

In one embodiment there is provided a purified antibody, for example a humanized antibody, in particular an antibody according to the invention, in substantially purified form, in particular free or substantially free of endotoxin and/or host cell protein or DNA.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

### Therapeutic Use of the Antibodies

The antibodies of the invention, formulations, or pharmaceutical compositions thereof may be administered for prophylactic and/or therapeutic treatments.

The present invention provides an anti-TREM1 antibody of the invention or pharmaceutical composition thereof for use as a medicament.

In prophylactic applications, antibodies, formulations, or compositions are administered to a subject at risk of a disorder or condition as described herein, in an amount sufficient to prevent or reduce the subsequent effects of the condition or one or more of its symptoms.

In therapeutic applications, the antibodies are administered to a subject already suffering from a disorder or condition as described herein, in an amount sufficient to cure, alleviate or partially arrest the condition or one or more of its symptoms. Such therapeutic treatment may result in a decrease in severity of disease symptoms, or an increase in frequency or duration of symptom-free periods.

The subjects to be treated can be animals. Preferably, the pharmaceutical compositions according to the present invention are adapted for administration to human subjects.

The present invention provides a method of treating a disorder or condition as described herein in a subject in need thereof, the method comprising administering to the subject an antibody according to the present invention. Such antibody is administered in a therapeutically effective amount.

The present invention also provides an antibody of the invention for use in the treatment of a disorder or condition as described herein.

### Therapeutic Indications

Antibodies of the present invention may be used in treating, preventing or ameliorating any condition that is associated with TREM1 activity; for example, any condition which results in whole or in part from signaling through TREM1.

TREM1 and its multiple pathways have been implicated in a number of neurological, neurodevelopmental, psychiatric, systemic and autoimmune inflammatory conditions. Some examples of the conditions that can treated using the antibodies and the compositions of the present invention include amyotrophic lateral sclerosis, Alzheimer's disease (AD), Parkinson's disease (PD), tauopathy disease, dementia, frontotemporal dementia, vascular dementia, mixed dementia, multiple system atrophy, epilepsy including Tuberous Sclerosis Complex and Focal Cortical Dysplasia, Huntington's disease, spinal cord injury, traumatic brain injury, chronic traumatic encephalopathy, ischemic stroke, multiple sclerosis, autoimmune neuritis, schizophrenia, autism spectrum disorders, major depressive disorders, bipolar disorder, hereditary conditions, or any combination thereof.

The antibodies and compositions of the present invention can be used to treat neurological disorders. More specifically said neurological disorder is amyotrophic lateral sclerosis (ALS) or Alzheimer's disease.

### Diagnostic Use of the Antibodies and Antigen-Binding Fragments Thereof

The present invention also provides the use of the antibodies of the present invention as diagnostically active agents or in diagnostic assays, for example, for diagnosing a disease or its severity.

The diagnosis may preferably be performed on biological samples. A “biological sample” encompasses a variety of sample types obtained from an individual and can be used in a diagnostic or monitoring assay. The definition encompasses cerebrospinal fluid, blood such as plasma and serum, and other liquid samples of biological origin such as urine and saliva, solid tissue samples such as a biopsy specimen or tissue cultures or cells derived therefrom and the progeny thereof The definition also includes samples that have been manipulated in any way after their procurement, such as by treatment with reagents, solubilization, or enrichment for certain components, such as polynucleotides.

Diagnostic testing may preferably be performed on biological samples which are not in contact with the human or animal body. Such diagnostic testing is also referred to as in vitro testing. In vitro diagnostic testing may rely on an in vitro method of detecting of TREM1 in a biological sample, which has been obtained from a subject.

### Pharmaceutical and Diagnostic Compositions

An antibody of the invention may be provided in a pharmaceutical composition. The pharmaceutical composition will normally be sterile and may additionally comprise a pharmaceutically acceptable adjuvant and/or carrier.

As the antibodies of the present invention are useful in the treatment, diagnosis and/or prophylaxis of a disorder or condition as described herein, the present invention also provides for a pharmaceutical or diagnostic composition comprising an antibody or antigen-binding fragment thereof according to the present invention in combination with one or more of a pharmaceutically acceptable carrier, excipient or diluent.

In particular the antibody or antigen-binding fragment thereof is provided as a pharmaceutical composition comprising one or more of a pharmaceutically acceptable excipient, diluent or carrier.

These compositions may comprise, in addition to the therapeutically active ingredient(s), a pharmaceutically acceptable excipient, carrier, diluent, buffer, stabilizer or other materials well known to those skilled in the art. Such materials should be non-toxic and should not interfere with the efficacy of the active ingredient.

Also provided are compositions, including pharmaceutical formulations, comprising an anti-TREM1 antibody of the invention, or polynucleotides comprising sequences encoding an antibody of the invention. In certain embodiments, compositions comprise one or more antibodies of the invention, or one or more polynucleotides comprising sequences encoding one or more antibodies of the invention. These compositions may further comprise suitable carriers, such as pharmaceutically acceptable excipients and/or adjuvants including buffers, which are well known in the art.

Pharmaceutical compositions of an antibody of the present invention are prepared by mixing such antibody having the desired degree of purity with one or more optional pharmaceutically acceptable carriers in the form of lyophilized formulations or aqueous solutions.

Examples of the techniques and protocols mentioned above can be found in Remington's Pharmaceutical Sciences, 20th Edition, 2000, pub. Lippincott, Williams & Wilkins.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and 2006/0104968. In one aspect, a sHASEGP is combined with one or more additional glycosaminoglycanases such as chondroitinases.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be also prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

The pharmaceutical compositions of the invention may include one or more pharmaceutically acceptable salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Pharmaceutical compositions typically must be sterile and stable under the conditions of manufacture and storage. The composition can be formulated as a solution, microemulsion, liposome, or other ordered structure suitable to high drug concentration.

In one embodiment, the antibody of the present invention is the sole active ingredient. In another embodiment, an antibody of the present invention is in combination with one or more additional active ingredients. Alternatively, the pharmaceutical compositions comprise the antibody of the present invention which is the sole active ingredient and it may be administered individually to a patient in combination (e.g. simultaneously, sequentially or separately) with other agents, drugs or hormones.

The precise nature of the carrier or other material may depend on the route of administration, e.g. oral, intravenous, cutaneous or subcutaneous, nasal, intramuscular and intraperitoneal routes. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Solutions for intravenous administration or infusion may contain as camer, for example, sterile water or preferably they may be in the form of sterile, aqueous, isotonic saline solutions.

Preferably, the pharmaceutical or diagnostic composition comprises a humanized antibody according to the present invention.

### Therapeutically Effective Amount and Dosage Determination

The antibodies and pharmaceutical compositions according to the present invention may be administered suitably to a patient to identify the therapeutically effective amount required. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Compositions may be conveniently presented in unit dose forms containing a predetermined amount of an active agent of the disclosure per dose. Dose ranges and regimens for any of the embodiments described herein include, but are not limited to, dosages ranging from 1 mg-1000 mg unit doses.

A suitable dosage of an antibody or pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01 μg/kg to about 1000 mg/kg body weight, typically from about 0.1 μg/kg to about 100 mg/kg body weight, of the patient to be treated.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

### Administration of Pharmaceutical Compositions or Formulations

The antibodies described herein or formulations or compositions thereof may be administered for prophylactic and/or therapeutic treatments.

An antibody or pharmaceutical composition of the invention may be administered via one or more routes of administration using one or more of a variety of methods known in the art. As will be appreciated by the skilled person, the route and/or mode of administration will vary depending upon the desired results. Examples of routes of administration for the antibodies or pharmaceutical compositions of the invention include intravenous, intramuscular, intradermal, intraocular, intraperitoneal, subcutaneous, spinal or other parenteral routes of administration, for example by injection or infusion. Alternatively, the antibody or pharmaceutical composition of the invention may be administered via a non-parenteral route, such as a topical, epidermal or mucosal route of administration. The antibody or pharmaceutical composition of the invention may be for oral administration.

Suitable forms for administration include forms suitable for parenteral administration, e.g. by injection or infusion, for example by bolus injection or continuous infusion, in intravenous, inhalable or sub-cutaneous form. Where the product is for injection or infusion, it may take the form of a suspension, solution or emulsion in an oily or aqueous vehicle and it may contain additional agents, such as suspending, preservative, stabilizing and/or dispersing agents. Alternatively, the antibody or antigen-binding fragment thereof according to the invention may be in dry form, for reconstitution before use with an appropriate sterile liquid. Solid forms suitable for solution in, or suspension in, liquid vehicles prior to injection may also be prepared.

Once formulated, the pharmaceutical compositions of the invention can be administered directly to the subject. Accordingly, provided herein is the use of an antibody or an antigen-binding fragment thereof according to the invention for the manufacture of a medicament.

### Articles of Manufacture and Kits

The present disclosure also provides kits comprising the anti-TREM1 antibodies of the present invention and instructions for use. The kit may further contain one or more additional reagents, such as an additional therapeutic or prophylactic agent as discussed above.

The present invention provides use of an antibody according to the invention or pharmaceutical composition thereof for the manufacture of a medicament.

The present invention also provides use of an antibody of the present invention for the manufacture of a medicament for the treatment of a disorder or condition as described herein.

In certain embodiments, the article of manufacture or kit comprises a container containing one or more of the antibodies of the invention, or the compositions described herein. In certain embodiments, the article of manufacture or kit comprises a container containing nucleic acids(s) encoding one (or more) of the antibodies or the compositions described herein. In some embodiments, the kit includes a cell or cell line that produces an antibody as described herein.

In certain embodiments, the article of manufacture or kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treatment, prevention and/or diagnosis and may have a sterile access port. At least one agent in the composition is an antibody of the present invention. The label or package insert indicates that the composition is used for the treatment of a disorder or condition as described herein.

It should be noted that the above-mentioned embodiments illustrate rather than limit the invention, and that those skilled in the art will be able to design many alternative embodiments without departing from the scope of the claims. In the claims, any reference signs placed between parentheses shall not be construed as limiting the claim.

The sequences included in the present invention are shown in Tables 6 and 7:

TABLE 6
Sequences of TREM1

| | | SEQ |
|---|---|---|
| | | ID |
| Name | Sequence | NO |
| Human TREM1 | MRKTRLWGLLWMLFVSELRAATKLTEEKYELKEGQTLDVKCDYTLEKF | 1 |
| isoform 1 | ASSQKAWQIIRDGEMPKTLACTERPSKNSHPVQVGRIILEDYHDHGLL | |
| (Accession No. | RVRMVNLQVEDSGLYQCVIYQPPKEPHMLFDRIRLVVTKGFSGTPGSN | |
| NP_061113.1) | ENSTQNVYKIPPTTTKALCPLYTSPRTVTQAPPKSTADVSTPDSEINL | |
| SwissProt: Q9NP99 | TNVTDIIRVPVFNIVILLAGGFLSKSLVFSVLFAVTLRSFVP | |
| Human TREM1 | ATKLTEEKYELKEGQTLDVKCDYTLEKFASSQKAWQIIRDGEMPKTLA | 2 |
| isoform 1 (21-234) | CTERPSKNSHPVQVGRIILEDYHDHGLLRVRMVNLQVEDSGLYQCVIY | |
| Without signal | QPPKEPHMLFDRIRLVVTKGFSGTPGSNENSTQNVYKIPPTTTKALCP | |
| peptide | LYTSPRTVTQAPPKSTADVSTPDSEINLTNVTDIIRVPVFNIVILLAG | |
| | GFLSKSLVFSVLFAVTLRSFVP | |
| Human TREM1 | MRKTRLWGLLWMLFVSELRAATKLTEEKYELKEGQTLDVKCDYTLEKF | 3 |
| isoform 2 | ASSQKAWQIIRDGEMPKTLACTERPSKNSHPVQVGRIILEDYHDHGLL | |
| (Accession No. | RVRMVNLQVEDSGLYQCVIYQPPKEPHMLFDRIRLVVTKGFSGTPGSN | |
| NP_001229518.1) | ENSTQNVYKIPPTTTKALCPLYTSPRTVTQAPPKSTADVSTPDSEINL | |
| | TNVTDIIRYSFQVPGPLVWTLSPLFPSLCAERM | |
| Human TREM1 | MRKTRLWGLLWMLFVSELRAATKLTEEKYELKEGQTLDVKCDYTLEKF | 4 |
| isoform 3 | ASSQKAWQIIRDGEMPKTLACTERPSKNSHPVQVGRIILEDYHDHGLL | |
| (Accession No. | RVRMVNLQVEDSGLYQCVIYQPPKEPHMLFDRIRLVVTKGFRCSTLSF | |
| NP_001229519) | SWLVDS | |
| Cynomolgus TREM1 | MRKTRLWGLLWMLFVSELRATTELTEEKYEYKEGQTLEVKCDYALEKY | 5 |
| protein | ANSRKAWQKMEGKMPKILAKTERPSENSHPVQVGRITLEDYPDHGLLQ | |
| (XP_001082517) | VQMTNLQVEDSGLYQCVIYQHPKESHVLFNPICLVVTKGSSGTPGSSE | |
| | NSTQNVYRTPSTTAKALGPRYTSPRTVTQAPPESTVVVSTPGSEINLT | |
| | NVTDIIRVPVFNIVIIVAGGFLSKSLVFSVLFAVTLRSFGP | |
| PGLYRP1 | MSRRSMLLAWALPSLLRLGAAQETEDPACCSPIVPRNEWKALASECAQ | 6 |
| (NP_005082.1) | HLSLPLRYVVVSHTAGSSCNTPASCQQQARNVQHYHMKTLGWCDVGYN | |
| | FLIGEDGLVYEGRGWNFTGAHSGHLWNPMSIGISFMGNYMDRVPTPQA | |
| | IRAAQGLLACGVAQGALRSNYVLKGHRDVQRTLSPGNQLYHLIQNWPH | |
| | YRSP | |
| CID101904 | MRKTRLWGLLWMLFVSELRAATKLTEEKYELKEGQTLDVKCDYTLEKF | 7 |
| hTREM1_1-200-Avi- | ASSQKAWQIIRDGEMPKTLACTERPSKNSHPVQVGRIILEDYHDHGLL | |
| Tev-HKH | RVRMVNLQVEDSGLYQCVIYQPPKEPHMLFDRIRLVVTKGFSGTPGSN | |
| | ENSTQNVYKIPPTTTKALCPLYTSPRTVTQAPPKSTADVSTPDSEINL | |
| | TNVTDIIRLEGGGSGGSGGLNDIFEAQKIEWHENLYFQGGSHHHHHHK | |
| | AKGSKGKGSKKAGHHHHHHHHHH | |
| CID101953 | MRKTRLWGLLWMLFVSELRATTELTEEKYEYKEGQTLEVKCDYALEKY | 8 |
| CTREM1_1-201-Avi- | ANSRKAWQKMEGKMPKILAKTERPSENSHPVQVGRITLEDYPDHGLLQ | |
| Tev-HKH | VRMTNLQVEDSGLYQCVIYQHPKESHVLFNPICLVVTKGSSGTPGSSE | |
| | NSTQNVYRTPSTTAKALGPRYTSPRTVTQAPPESTVVVSTPGSEINLT | |
| | NVTDIIRVPLEGGGSGGSGGLNDIFEAQKIEWHENLYFQGGSHHHHHH | |
| | KAKGSKGKGSKKAGHHHHHHHHHH | |
| CID101907 hTREM1 | MGHHHHHHSGEVKPEVKPETHINLKVSDGSSEIFFKIKKTTPLRRLME | 9 |
| IgV (His-Smt- | AFAKRQGKEMDSLRFLYDGIRIQADQTPEDLDMEDNDIIEAHREQIGG | |
| TREM1_21-139) | ATKLTEEKYELKEGQTLDVKCDYTLEKFASSQKAWQIIRDGEMPKTLA | |
| | CTERPSKNSHPVQVGRIILEDYHDHGLLRVRMVNLQVEDSGLYQCVIY | |
| | QPPKEPHMLFDRIRLVVTKGFSG | |
| CID101951 | MSRRSMLLAWALPSLLRLGAAQETEDPACCSPIVPRNEWKALASECAQ | 10 |
| hPGLYRP1_1-196-His | HLSLPLRYVVVSHTAGSSCNTPASCQQQARNVQHYHMKTLGWCDVGYN | |
| | FLIGEDGLVYEGRGWNFTGAHSGHLWNPMSIGISFMGNYMDRVPTPQA | |
| | IRAAQGLLACGVAQGALRSNYVLKGHRDVQRTLSPGNQLYHLIQNWPH | |
| | YRSPHHHHHH | |

TABLE 7
Sequences of the 12172 antibody and related variants

| | | SEQ |
|---|---|---|
| | | ID |
| Name | Sequence | NO |
| CDRL1 | QASQNIGSDLA | 11 |
| CDRL2 | KAATLAS | 12 |
| CDRL3 | QQYYYGSAGADTDT | 13 |
| CDRH1 | GFSLSSYAMT | 14 |
| CDRH2 | IIYAGGSPSYASWAKG | 15 |
| CDRH3 | GTGDTVYTYFNI | 16 |
| Rabbit Ab 12172 VL | AVVLTQTASPVSAPVGGTVTIKCQASQNIGSDLAWYQQEPGQPPKLLI | 17 |
| region | YKAATLASGVPSRFKGSGSGTEFTLTISGVQCEDGATYYCQQYYYGSA | |
| | GADTDTFGGGTEVVVK | |
| Rabbit Ab 12172 VL | gccgtcgtgctgacccagactgcatcccccgtgtctgcacctgtggga | 18 |
| region | ggcacagtcaccatcaagtgccaggccagtcagaacattggtagcgac | |
| | ttagcctggtatcagcaggaaccagggcagccacccaagctcctgatc | |
| | tacaaggcagccactctggcatctggggtcccatcgcggttcaaaggc | |
| | agtggatctgggacagagttcactctcaccatcagtggcgtgcagtgt | |
| | gaagatggtgccacttactactgtcaacagtattattatggtagtgct | |
| | ggtgctgatacggatactttcggcggagggaccgaggtggtggtcaaa | |
| CID102770: Rabbit | AVVLTQTASPVSAPVGGTVTIKCQASQNIGSDLAWYQQEPGQPPKLLI | 19 |
| Ab 12172 light | YKAATLASGVPSRFKGSGSGTEFTLTISGVQCEDGATYYCQQYYYGSA | |
| chain (Fab) | GADTDTFGGGTEVVVKRTPVAPTVLIFPPAADQVATGTVTIVCVANKY | |
| | FPDVTVTWEVDGTTQTTGIENSKTPQNSADCTYNLSSTLTLTSTQYNS | |
| | HKEYTCKVTQGTTSVVQSFNRGDC | |
| Rabbit Ab 12172 | gccgtcgtgctgacccagactgcatcccccgtgtctgcacctgtggga | 20 |
| light chain | ggcacagtcaccatcaagtgccaggccagtcagaacattggtagcgac | |
| | ttagcctggtatcagcaggaaccagggcagccacccaagctcctgatc | |
| | tacaaggcagccactctggcatctggggtcccatcgcggttcaaaggc | |
| | agtggatctgggacagagttcactctcaccatcagtggcgtgcagtgt | |
| | gaagatggtgccacttactactgtcaacagtattattatggtagtgct | |
| | ggtgctgatacggatactttcggcggagggaccgaggtggtggtcaaa | |
| | cgtacgccagttgcacctactgtcctcatcttcccaccagctgctgat | |
| | caggtggcaactggaacagtcaccatcgtgtgtgtggcgaataaatac | |
| | tttcccgatgtcaccgtcacctgggaggtggatggcaccacccaaaca | |
| | actggcatcgagaacagtaaaacaccgcagaattctgcagattgtacc | |
| | tacaacctcagcagcactctgacactgaccagcacacagtacaacagc | |
| | cacaaagagtacacctgcaaggtgacccagggcacgacctcagtcgtc | |
| | cagagcttcaataggggtgactgt | |
| Rabbit Ab 12172 VH | QSLEESGGRLVTPGTPLTLTCTASGFSLSSYAMTWVRQAPGKGLEWIG | 21 |
| region | IIYAGGSPSYASWAKGRFTISKTSTTVDLKMTSPTTEDTATYFCARGT | |
| | GDTVYTYFNIWGPGTLVTVSS | |
| Rabbit Ab 12172 VH | cagtcgctggaggagtccgggggtcgcctggtcacgcctgggacaccc | 22 |
| region | ctgacactcacctgcacagcctctggattctccctcagtagctatgca | |
| | atgacctgggtccgccaggctccagggaaggggctggaatggatcgga | |
| | atcatttatgctggtggtagcccatcctacgcgagctgggcgaaaggc | |
| | cgattcaccatctccaaaacctcgaccacggtggatctgaaaatgacc | |
| | agtccgacaaccgaggacacggccacctatttctgtgccagagggact | |
| | ggtgatactgtttatacctactttaatatttggggcccaggcaccctg | |
| | gtcaccgtctcgagt | |
| CID120769: Rabbit | QSLEESGGRLVTPGTPLTLTCTASGFSLSSYAMTWVRQAPGKGLEWIG | 23 |
| Ab 12172 heavy | IIYAGGSPSYASWAKGRFTISKTSTTVDLKMTSPTTEDTATYFCARGT | |
| chain (Fab) | GDTVYTYFNIWGPGTLVTVSSGQPKAPSVFPLAPCCGDTPSSTVTLGC | |
| | LVKGYLPEPVTVTWNSGTLTNGVRTFPSVRQSSGLYSLSSVVSVTSSS | |
| | QPVTCNVAHPATNTKVDKTVAPSTCSKP | |
| Rabbit Ab 12172 | cagtcgctggaggagtccgggggtcgcctggtcacgcctgggacaccc | 24 |
| heavy chain (Fab) | ctgacactcacctgcacagcctctggattctccctcagtagctatgca | |
| | atgacctgggtccgccaggctccagggaaggggctggaatggatcgga | |
| | atcatttatgctggtggtagcccatcctacgcgagctgggcgaaaggc | |
| | cgattcaccatctccaaaacctcgaccacggtggatctgaaaatgacc | |
| | agtccgacaaccgaggacacggccacctatttctgtgccagagggact | |
| | ggtgatactgtttatacctactttaatatttggggcccaggcaccctg | |
| | gtcaccgtctcgagtgggcaacctaaggctccatcagtcttcccactg | |
| | gccccctgctgcggggacacacccagctccacggtgaccctgggctgc | |
| | ctggtcaaaggctacctcccggagccagtgaccgtgacctggaactcg | |
| | ggcaccctcaccaatggggtacgcaccttcccgtccgtccggcagtcc | |
| | tcaggcctctactcgctgagcagcgtggtgagcgtgacctcaagcagc | |
| | cagcccgtcacctgcaacgtggcccacccagccaccaacaccaaagtg | |
| | gacaagaccgttgcgccctcgacatgcagcaagccc | |
| 12172gL1 V-region | AVVLTQSPSFLSASVGDRVTITCQASQNIGSDLAWYQQKPGKAPKLLI | 25 |
| | YKAATLASGVPSRFSGSGSGTEFTLTISSLQPEDFATYYCQQYYYGSA | |
| | GADTDTFGGGTKVEIK | |
| 12172gL1 V-region | gccgtggtgctgactcagtccccctccttcctgagcgcctcagtggga | 26 |
| | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| 12172gL1 light | AVVLTQSPSFLSASVGDRVTITCQASQNIGSDLAWYQQKPGKAPKLLI | 27 |
| chain | YKAATLASGVPSRFSGSGSGTEFTLTISSLQPEDFATYYCQQYYYGSA | |
| | GADTDTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSFNRGEC | |
| 12172gL1 light | gccgtggtgctgactcagtccccctccttcctgagcgcctcagtggga | 28 |
| chain | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| | cgtacggtagcggccccatctgtcttcatcttcccgccatctgatgag | |
| | cagttgaaatctggaactgcctctgttgtgtgcctgctgaataacttc | |
| | tatcccagagaggccaaagtacagtggaaggtggataacgccctccaa | |
| | tcgggtaactcccaggagagtgtcacagagcaggacagcaaggacagc | |
| | acctacagcctcagcagcaccctgacgctgagcaaagcagactacgag | |
| | aaacacaaagtctacgcctgcgaagtcacccatcagggcctgagctcg | |
| | cccgtcacaaagagcttcaacaggggagagtgt | |
| 12172gL2 V-region | DIQLTQSPSFLSASVGDRVTITCQASQNIGSDLAWYQQKPGKAPKLLI | 29 |
| | YKAATLASGVPSRFSGSGSGTEFTLTISSLQPEDFATYYCQQYYYGSA | |
| | GADTDTFGGGTKVEIK | |
| 12172gL2 V-region | gacatccagctgactcagtccccctccttcctgagcgcctcagtggga | 30 |
| | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| 12172gL2 light | DIQLTQSPSFLSASVGDRVTITCQASQNIGSDLAWYQQKPGKAPKLLI | 31 |
| chain | YKAATLASGVPSRFSGSGSGTEFTLTISSLQPEDFATYYCQQYYYGSA | |
| | GADTDTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSFNRGEC | |
| 12172gL2 light | gacatccagctgactcagtccccctccttcctgagcgcctcagtggga | 32 |
| chain | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| | cgtacggtggccgctccctccgtgttcatcttcccaccctccgacgag | |
| | cagctgaagtccggcaccgcctccgtcgtgtgcctgctgaacaacttc | |
| | tacccccgcgaggccaaggtgcagtggaaggtggacaacgccctgcag | |
| | tccggcaactcccaggaatccgtcaccgagcaggactccaaggacagc | |
| | acctactccctgtcctccaccctgaccctgtccaaggccgactacgag | |
| | aagcacaaggtgtacgcctgcgaagtgacccaccagggcctgtccagc | |
| | cccgtgaccaagtccttcaaccggggcgagtgc | |
| 12172gL6 V-region | DIVLTQSPSFLSASVGDRVTITCQASQNIGSDLAWYQQKPGKAPKLLI | 33 |
| | YKAATLASGVPSRFSGSGSGTEFTLTISSLQPEDFATYYCQQYYYGSA | |
| | GADTDTFGGGTKVEIK | |
| 12172gL6 V-region | gacatcgtgctgactcagtccccctccttcctgagcgcctcagtggga | 34 |
| | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| 12172gL6 light | DIVLTQSPSFLSASVGDRVTITCQASQNIGSDLAWYQQKPGKAPKLLI | 35 |
| chain | YKAATLASGVPSRFSGSGSGTEFTLTISSLQPEDFATYYCQQYYYGSA | |
| | GADTDTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSFNRGEC | |
| 12172gL6 light | gacatcgtgctgactcagtccccctccttcctgagcgcctcagtggga | 36 |
| chain | gatcgcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| | cgtacggtggccgctccctccgtgttcatcttcccaccctccgacgag | |
| | cagctgaagtccggcaccgcctccgtcgtgtgcctgctgaacaacttc | |
| | tacccccgcgaggccaaggtgcagtggaaggtggacaacgccctgcag | |
| | tccggcaactcccaggaatccgtcaccgagcaggactccaaggacagc | |
| | acctactccctgtcctccaccctgaccctgtccaaggccgactacgag | |
| | aagcacaaggtgtacgcctgcgaagtgacccaccagggcctgtccagc | |
| | cccgtgaccaagtccttcaaccggggcgagtgc | |
| 12172gL9 V-region | DIQLTQSPSFLSASVGDSVTITCQASQNIGSDLAWYQQKPGKAPKLLI | 37 |
| | YKAATLASGVPSRFSGSGSGTEFTLTISSLQPEDFATYYCQQYYYGSA | |
| | GADTDTFGGGTKVEIK | |
| 12172gL9 V-region | gacatccagctgactcagtccccctccttcctgagcgcctcagtggga | 38 |
| | gatagcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| 12172gL9 light | DIQLTQSPSFLSASVGDSVTITCQASQNIGSDLAWYQQKPGKAPKLLI | 39 |
| chain | YKAATLASGVPSRFSGSGSGTEFTLTISSLQPEDFATYYCQQYYYGSA | |
| | GADTDTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSFNRGEC | |
| 12172gL9 light | gacatccagctgactcagtccccctccttcctgagcgcctcagtggga | 40 |
| chain | gatagcgtgaccatcacctgtcaagccagccagaacattgggtccgac | |
| | ttggcctggtatcagcagaagcctggcaaagcgccgaagctgctgatc | |
| | tacaaggccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| | cgtacggtagcggccccatctgtcttcatcttcccgccatctgatgag | |
| | cagttgaaatctggaactgcctctgttgtgtgcctgctgaataacttc | |
| | tatcccagagaggccaaagtacagtggaaggtggataacgccctccaa | |
| | tcgggtaactcccaggagagtgtcacagagcaggacagcaaggacagc | |
| | acctacagcctcagcagcaccctgacgctgagcaaagcagactacgag | |
| | aaacacaaagtctacgcctgcgaagtcacccatcagggcctgagctcg | |
| | cccgtcacaaagagcttcaacaggggagagtgt | |
| 12172gL11 V-region | DIQLTQSPSFLSASVGDRVTITCQASQNIGSDLAWYQQKPGKAPKLLI | 41 |
| | YSAATLASGVPSRFSGSGSGTEFTLTISSLQPEDFATYYCQQYYYGSA | |
| | GADTDTFGGGTKVEIK | |
| 12172gL11 V-region | gacatccagctgactcagtccccctccttcctgagcgcctcagtggga | 42 |
| | gatcgcgtgaccatcacctgtcaagccagccagaacattgggtccgac | |
| | ttggcctggtatcagcagaagcctggcaaagcgccgaagctgctgatc | |
| | tacagcgccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| 12172gL11 light | DIQLTQSPSFLSASVGDRVTITCQASQNIGSDLAWYQQKPGKAPKLLI | 43 |
| chain | YSAATLASGVPSRFSGSGSGTEFTLTISSLQPEDFATYYCQQYYYGSA | |
| | GADTDTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSFNRGEC | |
| 12172gL11 light | gacatccagctgactcagtccccctccttcctgagcgcctcagtggga | 44 |
| chain | gatcgcgtgaccatcacctgtcaagccagccagaacattgggtccgac | |
| | ttggcctggtatcagcagaagcctggcaaagcgccgaagctgctgatc | |
| | tacagcgccgctactctcgcgtcgggagtgccatcccggttttccggt | |
| | tcgggaagcgggaccgagttcacccttaccatttcctcgctccaaccg | |
| | gaggacttcgccacgtactactgccagcagtactactacggttcagcc | |
| | ggagcagacactgataccttcggcggaggcaccaaggtcgaaatcaag | |
| | cgtacggtagcggccccatctgtcttcatcttcccgccatctgatgag | |
| | cagttgaaatctggaactgcctctgttgtgtgcctgctgaataacttc | |
| | tatcccagagaggccaaagtacagtggaaggtggataacgccctccaa | |
| | tcgggtaactcccaggagagtgtcacagagcaggacagcaaggacagc | |
| | acctacagcctcagcagcaccctgacgctgagcaaagcagactacgag | |
| | aaacacaaagtctacgcctgcgaagtcacccatcagggcctgagctcg | |
| | cccgtcacaaagagcttcaacaggggagagtgt | |
| 12172gH1 V-region | EVQLVESGGGLVQPGGSLRLSCTASGFSLSSYAMTWVRQAPGKGLEWI | 45 |
| | GIIYAGGSPSYASWAKGRFTISKDSSKNTVYLQMNSLRAEDTAVYYCA | |
| | RGTGDTVYTYFNIWGQGTLVTVSS | |
| 12172gH1 V-region | gaagtgcagctcgtggagtcgggcggaggtctggtgcagcccggagga | 46 |
| | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH1 heavy | EVQLVESGGGLVQPGGSLRLSCTASGFSLSSYAMTWVRQAPGKGLEWI | 47 |
| chain (IgG4P) | GIIYAGGSPSYASWAKGRFTISKDSSKNTVYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH1 heavy | gaagtgcagctcgtggagtcgggcggaggtctggtgcagcccggagga | 48 |
| chain (IgG4P) | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH2 V-region | EVQLVESGGGLVQPGGSLRLSCAASGFSLSSYAMTWVRQAPGKGLEWV | 49 |
| | GIIYAGGSPSYASWAKGRFTISKDSSKNTVYLQMNSLRAEDTAVYYCA | |
| | RGTGDTVYTYFNIWGQGTLVTVSS | |
| 12172gH2 V-region | gaagtgcagctcgtggagtcgggcggaggtctggtgcagcccggagga | 50 |
| | agcctgcgcctgtcatgcgctgccagcgggttctcgctttcctcctac | |
| | gccatgacctgggtcagacaggcaccgggaaagggcctcgaatgggtc | |
| | ggcattatctatgcgggtggcagcccttcctacgcctcctgggctaaa | |
| | gggcggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH2 heavy | EVQLVESGGGLVQPGGSLRLSCAASGFSLSSYAMTWVRQAPGKGLEWV | 51 |
| chain (IgG4P) | GIIYAGGSPSYASWAKGRFTISKDSSKNTVYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH2 heavy | gaagtgcagctcgtggagtcgggcggaggtctggtgcagcccggagga | 52 |
| chain (IgG4P) | agcctgcgcctgtcatgcgctgccagcgggttctcgctttcctcctac | |
| | gccatgacctgggtcagacaggcaccgggaaagggcctcgaatgggtc | |
| | ggcattatctatgcgggtggcagcccttcctacgcctcctgggctaaa | |
| | gggcggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH4 V-region | EVQLVESGGGLVQPGGSLRLSCTASGFSLSSYAMTWVRQAPGKGLEWI | 53 |
| | GIIYAGGSPSYASWAKGRFTISRDSSKNTVYLQMNSLRAEDTAVYYCA | |
| | RGTGDTVYTYFNIWGQGTLVTVSS | |
| 12172gH4 V-region | gaagtgcagctcgtggagtcgggcggaggtctggtgcagcccggagga | 54 |
| | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH4 heavy | EVQLVESGGGLVQPGGSLRLSCTASGFSLSSYAMTWVRQAPGKGLEWI | 55 |
| chain (IgG4P) | GIIYAGGSPSYASWAKGRFTISRDSSKNTVYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH4 heavy | gaagtgcagctcgtggagtcgggcggaggtctggtgcagcccggagga | 56 |
| chain (IgG4P) | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH6 V-region | EVQLVESGGGLVQPGGSLRLSCTASGFSLSSYAMTWVRQAPGKGLEWI | 57 |
| | GIIYAGGSPSYASWAKGRFTISKDSSKNTLYLQMNSLRAEDTAVYYCA | |
| | RGTGDTVYTYFNIWGQGTLVTVSS | |
| 12172gH6 V-region | gaagtgcagctcgtggagtcgggcggaggtctggtgcagcccggagga | 58 |
| | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH6 heavy | EVQLVESGGGLVQPGGSLRLSCTASGFSLSSYAMTWVRQAPGKGLEWI | 59 |
| chain (IgG4P) | GIIYAGGSPSYASWAKGRFTISKDSSKNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH6 heavy | gaagtgcagctcgtggagtcgggcggaggtctggtgcagcccggagga | 60 |
| chain (IgG4P) | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH6 heavy | EVQLVESGGGLVQPGGSLRLSCTASGFSLSSYAMTWVRQAPGKGLEWI | 61 |
| chain (IgG4P F234A | GIIYAGGSPSYASWAKGRFTISKDSSKNTLYLQMNSLRAEDTAVYYCA | |
| L235A) | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEAAGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH6 heavy | gaagtgcagctcgtggagtcgggcggaggtctggtgcagcccggagga | 62 |
| chain (IgG4P F234A | agcctgcgcctgtcatgcaccgccagcgggttctcgctttcctcctac | |
| L235A) | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgaagccgcggggggaccgtcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH6 heavy | EVQLVESGGGLVQPGGSLRLSCTASGFSLSSYAMTWVRQAPGKGLEWI | 63 |
| chain (IgG1) | GIIYAGGSPSYASWAKGRFTISKDSSKNTLYLQMNSLRAEDTAVYYCA | |
| | RGTGDTVYTYFNIWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| 12172gH6 heavy | gaagtgcagctcgtggagtcgggcggaggtctggtgcagcccggagga | 64 |
| chain (IgG1) | agcctgcgcctgtcatgcaccgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatcggtc | |
| | ttccccctggcaccctcctccaagagcacctctgggggcacagcggcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacccagacctacatctgcaacgtgaatcacaag | |
| | cccagcaacaccaaggtggacaagaaagttgagcccaaatcttgtgac | |
| | aaaactcacacatgcccaccgtgcccagcacctgaactcctgggggga | |
| | ccgtcagtcttcctcttccccccaaaacccaaggacaccctcatgatc | |
| | tcccggacccctgaggtcacatgcgtggtggtggacgtgagccacgaa | |
| | gaccctgaggtcaagttcaactggtacgtggacggcgtggaggtgcat | |
| | aatgccaagacaaagccgcgggaggagcagtacaacagcacgtaccgt | |
| | gtggtcagcgtcctcaccgtcctgcaccaggactggctgaatggcaag | |
| | gagtacaagtgcaaggtctccaacaaagccctcccagcccccatcgag | |
| | aaaaccatctccaaagccaaagggcagccccgagaaccacaggtgtac | |
| | accctgcccccatcccgggatgagctgaccaagaaccaggtcagcctg | |
| | acctgcctggtcaaaggcttctatcccagcgacatcgccgtggagtgg | |
| | gagagcaatgggcagccggagaacaactacaagaccacgcctcccgtg | |
| | ctggactccgacggctccttcttcctctacagcaagctcaccgtggac | |
| | aagagcaggtggcagcaggggaacgtcttctcatgctccgtgatgcat | |
| | gaggctctgcacaaccactacacgcagaagagcctctccctgtctccg | |
| | ggtaaa | |
| 12172gH6 heavy | EVQLVESGGGLVQPGGSLRLSCTASGFSLSSYAMTWVRQAPGKGLEWI | 65 |
| chain (IgG1 L234A | GIIYAGGSPSYASWAKGRFTISKDSSKNTLYLQMNSLRAEDTAVYYCA | |
| L235A) | RGTGDTVYTYFNIWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| 12172gH6 heavy | gaagtgcagctcgtggagtcgggcggaggtctggtgcagcccggagga | 66 |
| chain (IgG1 L234A | agcctgcgcctgtcatgcaccgccagcgggttctcgctttcctcctac | |
| L235A) | gccatgacctgggtcagacaggcaccgggaaagggcctcgaatggatt | |
| | ggcattatctatgcgggggcagcccttcctacgcctcctgggctaaa | |
| | gggcggttcaccatctcgaaggactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccctccgtg | |
| | ttcccgctcgctccatcatcgaagtctaccagcggaggcactgcggct | |
| | ctcggttgcctcgtgaaggactacttcccggagccggtgaccgtgtcg | |
| | tggaacagcggagccctgaccagcggggtgcacacctttccggccgtc | |
| | ttgcagtcaagcggcctttactccctgtcatcagtggtgactgtcccg | |
| | tccagctcattgggaacccaaacctacatctgcaatgtgaatcacaaa | |
| | cctagcaacaccaaggttgacaagaaagtcgagcccaaatcgtgtgac | |
| | aagactcacacttgtccgccgtgcccggcacccgaagccgcaggaggt | |
| | cccagcgtctttctgttccctccaaagccgaaagacacgctgatgatc | |
| | tcccgcaccccggaggtcacttgcgtggtcgtggacgtgtcacatgag | |
| | gacccagaggtgaagttcaattggtacgtggatggcgtcgaagtccac | |
| | aatgccaaaactaagcccagagaagaacagtacaattcgacctaccgc | |
| | gtcgtgtccgtgctcacggtgttgcatcaggattggctgaacgggaag | |
| | gaatacaagtgcaaagtgtccaacaaggcgctgccggcaccgatcgag | |
| | aaaactatctccaaagcgaagggacagcctagggaacctcaagtctac | |
| | acgctgccaccatcacgggatgaactgactaagaatcaagtctcactg | |
| | acttgtctggtgaaggggttttaccctagcgacattgccgtggagtgg | |
| | gaatccaacggccagccagagaacaactacaagactacccctccagtg | |
| | ctcgactcggatggatcgttcttcctttactcgaagctcaccgtggat | |
| | aagtcccggtggcagcagggaaacgtgttctcctgctcggtgatgcat | |
| | gaagccctccataaccactatacccaaaagtcgctgtccctgtcgccg | |
| | ggaaag | |
| 12172gH8 V-region | EVQLVESGGGLVQPGGSLRLSCAASGFSLSSYAMTWVRQAPGKGLEWI | 67 |
| | GIIYAGGSPSYASWAKGRFTISKDSSKNTVYLQMNSLRAEDTAVYYCA | |
| | RGTGDTVYTYFNIWGQGTLVTVSS | |
| 12172gH8 V-region | gaagtgcagctcgtggagtcgggcggaggtctggtgcagcccggagga | 68 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH8 heavy | EVQLVESGGGLVQPGGSLRLSCAASGFSLSSYAMTWVRQAPGKGLEWI | 69 |
| chain (IgG4P) | GIIYAGGSPSYASWAKGRFTISKDSSKNTVYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH8 heavy | gaagtgcagctcgtggagtcgggcggaggtctggtgcagcccggagga | 70 |
| chain (IgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggactcctccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH9 V-region | EVQLVESGGGLVQPGGSLRLSCAASGFSLSSYAMTWVRQAPGKGLEWI | 71 |
| | GIIYAGGSPSYASWAKGRFTISRDNSKNTVYLQMNSLRAEDTAVYYCA | |
| | RGTGDTVYTYFNIWGQGTLVTVSS | |
| 12172gH9 V-region | gaagtgcagctcgtggagtcgggcggaggtctggtgcagcccggagga | 72 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgacaattccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH9 heavy | EVQLVESGGGLVQPGGSLRLSCAASGFSLSSYAMTWVRQAPGKGLEWI | 73 |
| chain (IgG4P) | GIIYAGGSPSYASWAKGRFTISRDNSKNTVYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH9 heavy | gaagtgcagctcgtggagtcgggcggaggtctggtgcagcccggagga | 74 |
| chain (IgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgacaattccaagaacacggtgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH10 V-region | EVQLVESGGGLVQPGGSLRLSCAASGFSLSSYAMTWVRQAPGKGLEWI | 75 |
| | GIIYAGGSPSYASWAKGRFTISRDSSKNTLYLQMNSLRAEDTAVYYCA | |
| | RGTGDTVYTYFNIWGQGTLVTVSS | |
| 12172gH10 V-region | gaagtgcagctcgtggagtcgggcggaggtctggtgcagcccggagga | 76 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggggttcaccatctcgcgcgactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH10 heavy | EVQLVESGGGLVQPGGSLRLSCAASGFSLSSYAMTWVRQAPGKGLEWI | 77 |
| chain (IgG4P) | GIIYAGGSPSYASWAKGRFTISRDSSKNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH10 heavy | gaagtgcagctcgtggagtcgggcggaggtctggtgcagcccggagga | 78 |
| chain (IgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgactcctccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH11 V-region | EVQLVESGGGLVQPGGSLRLSCAASGFSLSSYAMTWVRQAPGKGLEWI | 79 |
| | GIIYAGGSPSYASWAKGRFTISKDNSKNTLYLQMNSLRAEDTAVYYCA | |
| | RGTGDTVYTYFNIWGQGTLVTVSS | |
| 12172gH11 V-region | gaagtgcagctcgtggagtcgggcggaggtctggtgcagcccggagga | 80 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH11 heavy | EVQLVESGGGLVQPGGSLRLSCAASGFSLSSYAMTWVRQAPGKGLEWI | 81 |
| chain (IgG4P) | GIIYAGGSPSYASWAKGRFTISKDNSKNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH11 heavy | gaagtgcagctcgtggagtcgggcggaggtctggtgcagcccggagga | 82 |
| chain (IgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccctccgtg | |
| | ttccctctggccccttgctcccggtccacctccgagtctaccgccgct | |
| | ctgggctgcctggtcaaggactacttccccgagcccgtgacagtgtcc | |
| | tggaactctggcgccctgacctccggcgtgcacaccttccctgccgtg | |
| | ctgcagtcctccggcctgtactccctgtcctccgtcgtgaccgtgccc | |
| | tcctccagcctgggcaccaagacctacacctgtaacgtggaccacaag | |
| | ccctccaacaccaaggtggacaagcgggtggaatctaagtacggccct | |
| | ccctgccccccctgccctgcccctgaatttctgggcggaccttccgtg | |
| | ttcctgttccccccaaagcccaaggacaccctgatgatctcccggacc | |
| | cccgaagtgacctgcgtggtggtggacgtgtcccaggaagatcccgag | |
| | gtccagttcaattggtacgtggacggcgtggaagtgcacaatgccaag | |
| | accaagcccagagaggaacagttcaactccacctaccgggtggtgtcc | |
| | gtgctgaccgtgctgcaccaggactggctgaacggcaaagagtacaag | |
| | tgcaaggtgtccaacaagggcctgccctccagcatcgaaaagaccatc | |
| | tccaaggccaagggccagccccgcgagccccaggtgtacaccctgccc | |
| | cctagccaggaagagatgaccaagaaccaggtgtccctgacctgtctg | |
| | gtcaagggcttctacccctccgacattgccgtggaatgggagtccaac | |
| | ggccagcccgagaacaactacaagaccaccccccctgtgctggacagc | |
| | gacggctccttcttcctgtactctcggctgaccgtggacaagtcccgg | |
| | tggcaggaaggcaacgtcttctcctgctccgtgatgcacgaggccctg | |
| | cacaaccactacacccagaagtccctgtccctgagcctgggcaag | |
| 12172gH11 heavy | EVQLVESGGGLVQPGGSLRLSCAASGFSLSSYAMTWVRQAPGKGLEWI | 83 |
| chain (IgG4P F234A | GIIYAGGSPSYASWAKGRFTISKDNSKNTLYLQMNSLRAEDTAVYYCA | |
| L235A) | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEAAGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH11 heavy | gaagtgcagctcgtggagtcgggcggaggtctggtgcagcccggagga | 84 |
| chain (IgG4P F234A | agcctgcgcctgtcatgcgctgccagcgggttctcgctttcctcctac | |
| L235A) | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgaagccgcggggggaccgtcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH11 heavy | EVQLVESGGGLVQPGGSLRLSCAASGFSLSSYAMTWVRQAPGKGLEWI | 85 |
| chain (IgG1) | GIIYAGGSPSYASWAKGRFTISKDNSKNTLYLQMNSLRAEDTAVYYCA | |
| | RGTGDTVYTYFNIWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| 12172gH11 heavy | gaagtgcagctcgtggagtcgggcggaggtctggtgcagcccggagga | 86 |
| chain (IgG1) | agcctgcgcctgtcatgcgctgccagcgggttctcgctttcctcctac | |
| | gccatgacctgggtcagacaggcaccgggaaagggcctcgaatggatt | |
| | ggcattatctatgcgggggcagcccttcctacgcctcctgggctaaa | |
| | gggcggttcaccatctcgaaggacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatcggtc | |
| | ttccccctggcaccctcctccaagagcacctctgggggcacagcggcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacccagacctacatctgcaacgtgaatcacaag | |
| | cccagcaacaccaaggggacaagaaagttgagcccaaatcttgtgac | |
| | aaaactcacacatgcccaccgtgcccagcacctgaactcctgggggga | |
| | ccgtcagtcttcctcttccccccaaaacccaaggacaccctcatgatc | |
| | tcccggacccctgaggtcacatgcgtggtggtggacgtgagccacgaa | |
| | gaccctgaggtcaagttcaactggtacgtggacggcgtggaggtgcat | |
| | aatgccaagacaaagccgcgggaggagcagtacaacagcacgtaccgt | |
| | gtggtcagcgtcctcaccgtcctgcaccaggactggctgaatggcaag | |
| | gagtacaagtgcaaggtctccaacaaagccctcccagcccccatcgag | |
| | aaaaccatctccaaagccaaagggcagccccgagaaccacaggtgtac | |
| | accctgcccccatcccgggatgagctgaccaagaaccaggtcagcctg | |
| | acctgcctggtcaaaggcttctatcccagcgacatcgccgtggagtgg | |
| | gagagcaatgggcagccggagaacaactacaagaccacgcctcccgtg | |
| | ctggactccgacggctccttcttcctctacagcaagctcaccgtggac | |
| | aagagcaggtggcagcaggggaacgtcttctcatgctccgtgatgcat | |
| | gaggctctgcacaaccactacacgcagaagagcctctccctgtctccg | |
| | ggtaaa | |
| 12172gH11 heavy | EVQLVESGGGLVQPGGSLRLSCAASGFSLSSYAMTWVRQAPGKGLEWI | 87 |
| chain (IgG1 L234A | GIIYAGGSPSYASWAKGRFTISKDNSKNTLYLQMNSLRAEDTAVYYCA | |
| L235A) | RGTGDTVYTYFNIWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| 12172gH11 heavy | gaagtgcagctcgtggagtcgggcggaggtctggtgcagcccggagga | 88 |
| chain (IgG1 L234A | agcctgcgcctgtcatgcgctgccagcgggttctcgctttcctcctac | |
| L235A) | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccctccgtg | |
| | ttcccgctcgctccatcatcgaagtctaccagcggaggcactgcggct | |
| | ctcggttgcctcgtgaaggactacttcccggagccggtgaccgtgtcg | |
| | tggaacagcggagccctgaccagcggggtgcacacctttccggccgtc | |
| | ttgcagtcaagcggcctttactccctgtcatcagtggtgactgtcccg | |
| | tccagctcattgggaacccaaacctacatctgcaatgtgaatcacaaa | |
| | cctagcaacaccaaggttgacaagaaagtcgagcccaaatcgtgtgac | |
| | aagactcacacttgtccgccgtgcccggcacccgaagccgcaggaggt | |
| | cccagcgtctttctgttccctccaaagccgaaagacacgctgatgatc | |
| | tcccgcaccccggaggtcacttgcgtggtcgtggacgtgtcacatgag | |
| | gacccagaggtgaagttcaattggtacgtggatggcgtcgaagtccac | |
| | aatgccaaaactaagcccagagaagaacagtacaattcgacctaccgc | |
| | gtcgtgtccgtgctcacggtgttgcatcaggattggctgaacgggaag | |
| | gaatacaagtgcaaagtgtccaacaaggcgctgccggcaccgatcgag | |
| | aaaactatctccaaagcgaagggacagcctagggaacctcaagtctac | |
| | acgctgccaccatcacgggatgaactgactaagaatcaagtctcactg | |
| | acttgtctggtgaaggggttttaccctagcgacattgccgtggagtgg | |
| | gaatccaacggccagccagagaacaactacaagactacccctccagtg | |
| | ctcgactcggatggatcgttcttcctttactcgaagctcaccgtggat | |
| | aagtcccggtggcagcagggaaacgtgttctcctgctcggtgatgcat | |
| | gaagccctccataaccactatacccaaaagtcgctgtccctgtcgccg | |
| | ggaaag | |
| 12172gH12 V-region | EVQLVESGGGLVQPGGSLRLSCAASGFSLSSYAMTWVRQAPGKGLEWI | 89 |
| | GIIYAGGSPSYASWAKGRFTISRDNSKNTLYLQMNSLRAEDTAVYYCA | |
| | RGTGDTVYTYFNIWGQGTLVTVSS | |
| 12172gH12 V-region | gaagtgcagctcgtggagtcgggcggaggtctggtgcagcccggagga | 90 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH12 heavy | EVQLVESGGGLVQPGGSLRLSCAASGFSLSSYAMTWVRQAPGKGLEWI | 91 |
| chain (IgG4P) | GIIYAGGSPSYASWAKGRFTISRDNSKNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH12 heavy | gaagtgcagctcgtggagtcgggcggaggtctggtgcagcccggagga | 92 |
| chain (IgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgcgcgacaattccaagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH26 V-region | EVQLVESGGGLVQPGGSLRLSCAASGFSLSSYAMTWVRQAPGKGLEWI | 93 |
| | GIIYAGGSPSYASWAKGRFTISKDNSSNTLYLQMNSLRAEDTAVYYCA | |
| | RGTGDTVYTYFNIWGQGTLVTVSS | |
| 12172gH26 V-region | gaagtgcagctcgtggagtcgggcggaggtctggtgcagcccggagga | 94 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccagcaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH26 heavy | EVQLVESGGGLVQPGGSLRLSCAASGFSLSSYAMTWVRQAPGKGLEWI | 95 |
| chain (hIgG4P) | GIIYAGGSPSYASWAKGRFTISKDNSSNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH26 heavy | gaagtgcagctcgtggagtcgggcggaggtctggtgcagcccggagga | 96 |
| chain (hIgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggggcagcccttcctacgcctcctgggctaaa | |
| | gggcggttcaccatctcgaaggacaattccagcaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH48 V-region | EVQLVESGGGLVQPGGSLRLSCAASGFSLSSYAMTWVRQAPGKGLEWI | 97 |
| | GIIYAGGSPSYASWAKGRFTISKDNSQNTLYLQMNSLRAEDTAVYYCA | |
| | RGTGDTVYTYFNIWGQGTLVTVSS | |
| 12172gH48 V-region | gaagtgcagctcgtggagtcgggcggaggtctggtgcagcccggagga | 98 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggggcagcccttcctacgcctcctgggctaaa | |
| | gggcggttcaccatctcgaaggacaattcccagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH48 heavy | EVQLVESGGGLVQPGGSLRLSCAASGFSLSSYAMTWVRQAPGKGLEWI | 99 |
| chain (hIgG4P) | GIIYAGGSPSYASWAKGRFTISKDNSQNTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH48 heavy | gaagtgcagctcgtggagtcgggcggaggtctggtgcagcccggagga | 100 |
| chain (hIgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattcccagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| 12172gH49 V-region | EVQLVESGGGLVQPGGSLRLSCAASGFSLSSYAMTWVRQAPGKGLEWI | 101 |
| | GIIYAGGSPSYASWAKGRFTISKDNSENTLYLQMNSLRAEDTAVYYCA | |
| | RGTGDTVYTYFNIWGQGTLVTVSS | |
| 12172gH49 V-region | gaagtgcagctcgtggagtcgggcggaggtctggtgcagcccggagga | 102 |
| | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccgagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagc | |
| 12172gH49 heavy | EVQLVESGGGLVQPGGSLRLSCAASGFSLSSYAMTWVRQAPGKGLEWI | 103 |
| chain (hIgG4P) | GIIYAGGSPSYASWAKGRFTISKDNSENTLYLQMNSLRAEDTAVYYCA | |
| | RGTGDTVYTYFNIWGQGTLVTVSSASTKGPSVFPLAPCSRSTSESTAA | |
| | LGCLVKDYFPEPVTVSWNSGALTSGVHTFPAVLQSSGLYSLSSVVTVP | |
| | SSSLGTKTYTCNVDHKPSNTKVDKRVESKYGPPCPPCPAPEFLGGPSV | |
| | FLFPPKPKDTLMISRTPEVTCVVVDVSQEDPEVQFNWYVDGVEVHNAK | |
| | TKPREEQFNSTYRVVSVLTVLHQDWLNGKEYKCKVSNKGLPSSIEKTI | |
| | SKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEAL | |
| | HNHYTQKSLSLSLGK | |
| 12172gH49 heavy | gaagtgcagctcgtggagtcgggcggaggtctggtgcagcccggagga | 104 |
| chain (hIgG4P) | agcctgcgcctgtcatgcgctgccagcgggttctcgctttcctcctac | |
| | gccatgacctgggtcagacaggcaccgggaaagggcctcgaatggatt | |
| | ggcattatctatgcgggtggcagcccttcctacgcctcctgggctaaa | |
| | gggcggttcaccatctcgaaggacaattccgagaacacgctgtacttg | |
| | caaatgaactcactgagggccgaggacactgcggtgtactactgtgcc | |
| | cggggaactggagataccgtgtacacctactttaacatctggggccag | |
| | ggaaccctggtcactgtctcgagcgcttctacaaagggcccatccgtc | |
| | ttccccctggcgccctgctccaggagcacctccgagagcacagccgcc | |
| | ctgggctgcctggtcaaggactacttccccgaaccggtgacggtgtcg | |
| | tggaactcaggcgccctgaccagcggcgtgcacaccttcccggctgtc | |
| | ctacagtcctcaggactctactccctcagcagcgtggtgaccgtgccc | |
| | tccagcagcttgggcacgaagacctacacctgcaacgtagatcacaag | |
| | cccagcaacaccaaggtggacaagagagttgagtccaaatatggtccc | |
| | ccatgcccaccatgcccagcacctgagttcctggggggaccatcagtc | |
| | ttcctgttccccccaaaacccaaggacactctcatgatctcccggacc | |
| | cctgaggtcacgtgcgtggtggtggacgtgagccaggaagaccccgag | |
| | gtccagttcaactggtacgtggatggcgtggaggtgcataatgccaag | |
| | acaaagccgcgggaggagcagttcaacagcacgtaccgtgtggtcagc | |
| | gtcctcaccgtcctgcaccaggactggctgaacggcaaggagtacaag | |
| | tgcaaggtctccaacaaaggcctcccgtcctccatcgagaaaaccatc | |
| | tccaaagccaaagggcagccccgagagccacaggtgtacaccctgccc | |
| | ccatcccaggaggagatgaccaagaaccaggtcagcctgacctgcctg | |
| | gtcaaaggcttctaccccagcgacatcgccgtggagtgggagagcaat | |
| | gggcagccggagaacaactacaagaccacgcctcccgtgctggactcc | |
| | gacggctccttcttcctctacagcaggctaaccgtggacaagagcagg | |
| | tggcaggaggggaatgtcttctcatgctccgtgatgcatgaggctctg | |
| | cacaaccactacacacagaagagcctctccctgtctctgggtaaa | |
| Human IGKV1-9 | DIQLTQSPSFLSASVGDRVTITCRASQGISSYLAWYQQKPGKAPKLLI | 105 |
| IGKJ4 acceptor | YAASTLQSGVPSRFSGSGSGTEFTLTISSLQPEDFATYYCQQLNSYPL | |
| framework | TFGGGTKVEIK | |
| Human IGKV1-9 | gacatccagttgacccagtctccatccttcctgtctgcatctgtagga | 106 |
| IGKJ4 acceptor | gacagagtcaccatcacttgccgggccagtcagggcattagcagttat | |
| framework | ttagcctggtatcagcaaaaaccagggaaagcccctaagctcctgatc | |
| | tatgctgcatccactttgcaaagtggggtcccatcaaggttcagcggc | |
| | agtggatctgggacagaattcactctcacaatcagcagcctgcagcct | |
| | gaagattttgcaacttattactgtcaacagcttaatagttaccctctc | |
| | actttcggcggagggaccaaggtggagatcaaa | |
| Human IGHV3-66 | EVQLVESGGGLVQPGGSLRLSCAASGFTVSSNYMSWVRQAPGKGLEWV | 107 |
| IGHJ4 acceptor | SVIYSGGSTYYADSVKGRFTISRDNSKNTLYLQMNSLRAEDTAVYYCA | |
| framework | RYFDYWGQGTLVTVSS | |
| Human IGHV3-66 | gaggtgcagctggtggagtctgggggaggcttggtccagcctgggggg | 108 |
| IGHJ4 acceptor | tccctgagactctcctgtgcagcctctggattcaccgtcagtagcaac | |
| framework | tacatgagctgggtccgccaggctccagggaaggggctggagtgggtc | |
| | tcagttatttatagcggtggtagcacatactacgcagactccgtgaag | |
| | ggcagattcaccatctccagagacaattccaagaacacgctgtatctt | |
| | caaatgaacagcctgagagccgaggacacggctgtgtattactgtgcg | |
| | agatactttgactactggggccaaggaaccctggtcaccgtctcctca | |

### EXAMPLES

### Example 1: Generation of TREM1 Proteins

Human TREM1 IgV-like domain fused with an N-terminal hexahistidine SUMO (small ubiquitin-related modifier) solubility tag (CID101907) was expressed in Escherichia coli BL21 (DE3). Bacteria were harvested by centrifugation, resuspended in 100 mM Tris pH 8, 300 mM NaCl, 250U Benzonase, 1 PI tab and lysed by sonication. The lysate was clarified by centrifugation at 42,000 RPM (Ti45), 4° C., 45 minutes and applied to a 5 ml HiTrap Ni Chelating Column. The column was washed with 100 mM Tris, pH 8, 300 mM NaCl for 5CV. The bound TREM1 proteins were eluted with a linear gradient 2-60% 100 mM Tris, pH 8, 300 mM NaCl, 500 mM Imidazole buffer for 15 CV, then 100% buffer B for 4CV. Fractions containing TREM1 were pooled, dialyzed (10 kDa MWCO) into 100 mM Tris pH 8, 300 mM NaCl, and digested with ULP-1 overnight at 4° C. The cleaved protein was applied to a 5 ml HiTrap Ni Chelating Column and the flow-through fractions were collected and concentrated in a Vivaspin PES Turbo, 10 kDa MWCO concentrator. A superdex s75 column (GE Healthcare) was then used to polish and buffer exchange the cleaved IgV protein into 100 mM Tris pH 8.0, 300 mM NaCl, 0.5 mM EDTA. The final protein concentration was determined by measuring 280 nm absorbance with a Nanodrop UV spectrometer. Protein purity was assessed by sodium dodecyl sulfate polyacrylamide gel electrophoresis (SDS-PAGE)

Both human (CID101904) and cyno (CID101953) TREM1 extracellular domain (ECD) containing the native N-terminal signal sequence and a C-terminal avidin affinity tag (AVI), TEV protease cleavage site, and a HKH affinity tag were expressed in mammalian HEK cells. The media was passed through tangential flow filtration (TFF) and applied to a HiTrap Ni Chelating 5 mL column and washed with 100 mM HEPES pH 7.0, 300 mM NaCl. The bound TREM1 proteins were eluted, respectively with 2-60% then 100% 100 mM HEPES pH 7.0, 300 mM NaCl, 500 mM Imidazole linear gradient over 4 CV. Fractions containing TREM1 were pooled and concentrated in a Vivaspin PES Turbo, 10 kDa MWCO concentrator. A sephacryl s300 column was then used to polish and buffer exchange the proteins into 50 mM HEPES pH 7.0, 250 mM NaCl. The final protein concentration and purity was assessed as previously described.

Human PGLYRP1 containing the native N-terminal signal sequence and a non-cleavable C-terminal his tag (CID101951) was expressed in mammalian HEK cells. The media was applied to a HiTrap Ni Excel 2×5 mL column and washed with 100 mM HEPES pH 7.0, 300 mM NaCl. The bound PGLYRP1 proteins were eluted, respectively with 0-60% then 100% 100 mM HEPES pH 7.0, 300 mM NaCl, 500 mM Imidazole linear gradient over 4 CV. Fractions containing PGLYRP1 were pooled and concentrated in a Vivaspin PES Turbo, 10 kDa MWCO concentrator. A superdex S200 column was then used to polish and buffer exchange the proteins into PBS pH 7.0. The final protein concentration and purity was assessed as previously described.

### Example 2. Generation and Selection of Therapeutic Anti-TREM1 Antibody 12172

One female New Zealand White rabbit was immunized sub-cutaneously with 3×10⁷ rabbit fibroblast cells transiently expressing human TREM1 on the cell surface. Cells were transfected via electroporation and expression of TREM1 was verified by flow cytometry using anti-TREM1 antibody (R&D FAB1278P). An equal volume of complete Freunds adjuvant was injected sub-cutaneous into the rabbit at a separate site at the same time as immunization with cells.

The rabbit was given two booster injections at 14 day intervals with the rabbit fibroblast cells transiently expressing human TREM1 on the cell surface. Heparinised bleeds (200 μl) were taken from the ear vein prior to each immunization. Sera was collected from the bleeds after spinning 10,000 rpm for 5 minutes in a bench top centrifuge and frozen down at −20° C. Termination occurred 14 days after the final boost with single cell suspensions of spleen, lymph node, bone marrow and peripheral blood mononuclear cells prepared and frozen in 10% DMSO/FCS at −80° C. until required for B cell discovery purposes. A bleed was also taken at termination and sera prepared as previously described.

Memory B cell cultures were set up using the method described by Tickle et al. (2015) in J Biomol Screen 20(4):492-7 and supernatants were first screened for their ability to bind human and cynomolgus TREM1 in a cell-based assay on the TTP Labtech Mirrorball system. Cell-based assays were a homogeneous multiplex assay using HEK 293 cells transiently transfected with either human TREM1 or cynomolgus TREM1 DNA, and counter screened against HEK 293 cells transiently transfected with irrelevant DNA. Cells were stained with either Vybrant™ DIO or DIL labelling (ThermoFisher) and a goat anti-rabbit Fc-AF647 conjugate as a reveal agent.

Approx. 7000 TREM1-specific positive hits were identified in the primary Mirrorball screens from a total of 20×200-plate B-cell culture experiments. Positive supernatants from this assay were then progressed for further characterization by profiling in BIAcore to estimate off-rate.

Wells with off-rates resulting in less than a 25% loss of binding during interrogation of a 300s disassociation step were progressed for V region gene recovery using the fluorescent foci method and single cell reverse transcription (RT) and PCR (RT-PCR).

Following reverse transcription (RT) and PCR of the picked cells, ‘transcriptionally active PCR’ (TAP) products encoding the antibodies' V regions were generated and used to transiently transfect HEK-293 cells. The resultant TAP supernatants, containing recombinant antibody, were tested for their ability to: bind human (CID101904; SEQ ID NO: 7) and cynomolgus (CID101953; SEQ ID NO: 8) TREM1 extracellular domain (generated as described in Example 1) by ELISA, bind to human sTREMI in the BIAcore with affinity of at least 1000 μM, and block PGLYRP1-mediated signaling in the THP1 monocyte TREM1/DAP12 NF-κB Luciferase reporter cell assay.

Functionality was assessed by the ability of antibodies to inhibit PGLYRP1/PGN mediated NF-κB signaling activation through human TREM1. To do this, THP1 monocyte TREM1/DAP12 NF-κB Luciferase reporter cells were used (generated at UCB). These cells stably express human TREM1, human DAP12 and a NF-κB luciferase reporter gene. PGLYRP1 complexed with soluble peptidoglycan from E. coli (PGN) was used as the TREM1 ligand, which induces NF-κB activation by binding to TREM1. PGN which does not bind to TREM1 also induces NF-κB activation, but to a lesser extent and through an alternative signaling pathway. Inhibition of luciferase activity demonstrates the functional blocking activity of antibodies in this system.

THP1 monocyte TREM1/DAP12 NF-κB Luciferase reporter cells were cultured in complete media containing selection antibiotics (RPMI+10% FBS+50 μM 2-mercaptoethanol+10 μg/ml blasticidin+1 μg/ml puromycin+200 μg/ml geneticin) using standard tissue culture techniques. Three days before assay set up, the cells were seeded at 10×10⁶ cells in 50 ml complete media (200,000 cells/ml) in a T175 flask, placed flat in the incubator. On the day of the assay, the cells were removed from the flask and transferred to a 50 ml falcon and centrifuged at 300×g for five minutes. Media was removed and the cells were resuspended in 5-10 ml of complete media and counted. Cells were then resuspended at 1×10⁶ cells/ml by adding cell suspension to complete media, and 10 μl/well was added to an assay plate (Corning #3570). Antibodies were serially diluted in complete media in a 384-well dilution plate (Greiner #781281). The serial dilution of antibodies was then transferred to the assay plate (10 μl/well) and the assay plate was incubated at 37° C./5% CO₂ for 1 hour. Recombinant human PGLYRP1 (R&D Systems #2590-PGB) was complexed with PGN (Invivogen #tlrl-ksspgn) for one hour at room temperature in sterile DPBS. After one hour, the solution was diluted in complete media, then transferred to the assay plate (10 μl/well) to a final assay concentration of 2.5 pg/ml PGLYRP1 and 10 μg/ml PGN. The plate controls (no antibody added) included PGLYRP1/PGN complex and PGN alone, as assay maximum and minimum values, respectively. The assay plate was then incubated at 37° C./5% CO₂ for 16 hours+2 hours. Following the incubation, luciferase activity was measured using the SteadyGlo Luciferase assay system (Promega #E2520). The Steady-Glo reagent was prepared according to the manufacturer's instructions and 30 μl/well was added to the assay plate. The plate was then centrifuged at 200×g for three minutes and then incubated at room temperature for a further two minutes so that the total incubation time with the SteadyGlo reagent was five minutes. Luminescence was then measured using a Synergy Neo 2 plate reader and the raw luminescence values were used to determine the relative percentage inhibition as compared to the control wells. 4PL curve fitting and the calculation of IC₅₀ values was performed using ActivityBase v9.4.

Heavy and light chain variable region gene pairs from interesting TAP products were then cloned as rabbit IgG antibodies and re-expressed in a HEK-293 transient expression system. In total 144 V regions were cloned. Recombinant cloned antibodies were then retested for their ability to bind human and cynomolgus TREM1 by ELISA, binding in the BIAcore and inhibition of PGLYRP1+ PGN-mediated signaling in the NF-κB luciferase reporter cell assay. Following characterization of the ligand binding site of known TREM1 ligand PGLYRP1 using a human TREM1 Alanine mutant array (the same approach as described further below for the TREM1-inhibiting antibodies), it was postulated that antibodies that bind to the same binding site regulate TREM1 function through direct ligand blocking. To identify alternative antibody binding sites on TREM1 which confer function, antibodies proven to inhibit TREM1 activity in the NF-κB luciferase reporter cell assay were assessed for epitope location using a human TREM1 Alanine mutant array.

Arrays of human TREM1 IgV domain mutant clones were produced. They consisted of either 58 clones each with three surface residues, in close proximity, mutated to alanine; 65 clones each with two surface residues, in close proximity, mutated to alanine; or 63 clones each with a single surface residue mutated to alanine. All arrays included the wild type human TREM1 clone. Sequences of the mutant human TREM1 array clones including the wild type are shown in Tables 8, 9, and 10.

TABLE 8
List of TREM1 protein sequences used to design the three-alanine mutant array

| | SEQ | |
|---|---|---|
| | ID | |
| ID | NO | TREM1 polypeptide sequence |
| 01_WT | 109 | MELRAATKLTEEKYELKEGQTLDVKCDYTLEKFASSQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 02_6 | 110 | MELRAAAKLTEEKYELKEGQTLDVKCDYTLEKFASSQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 03_6_7 | 111 | MELRAAAALTEEKYELKEGQTLDVKCDYTLEKFASSQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 04_6_7_9 | 112 | MELRAAAALAEEKYELKEGQTLDVKCDYTLEKFASSQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 05_7_8_9 | 113 | MELRAATAAAEEKYELKEGQTLDVKCDYTLEKFASSQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 06_9_10_ | 114 | MELRAATKLAAEKYELKEGQTLDVKCDYTLEKFASSQKAWQIIRDGEMPKTLACTERPSKNSHPV |
| 111 | | QVGRIILEDYHDHGLLRVRMVNLQVEDSGLYQCVIYQPPKEPHMLFARIRLVVTLE |
| 07_9_10_ | 115 | MELRAATKLAAAKYELKEGQTLDVKCDYTLEKFASSQKAWQIIRDGEMPKTLACTERPSKNSHPV |
| 11 | | QVGRIILEDYHDHGLLRVRMVNLQVEDSGLYQCVIYQPPKEPHMLFDRIRLVVTLE |
| 08_10_11_ | 116 | MELRAATKLTAAAYELKEGQTLDVKCDYTLEKFASSQKAWQIIRDGEMPKTLACTERPSKNSHPV |
| 12 | | QVGRIILEDYHDHGLLRVRMVNLQVEDSGLYQCVIYQPPKEPHMLFDRIRLVVTLE |
| 09_11_12_ | 117 | MELRAATKLTEAAAELKEGQTLDVKCDYTLEKFASSQKAWQIIRDGEMPKTLACTERPSKNSHPV |
| 13 | | QVGRIILEDYHDHGLLRVRMVNLQVEDSGLYQCVIYQPPKEPHMLFDRIRLVVTLE |
| 10_16_118_ | 118 | MELRAATKLTEEKYELAEGQTLDVKCDYTLEKFASSQKAWQIIRDGEMPKTLACTERPSKNSHPV |
| 119 | | QVGRIILEDYHDHGLLRVRMVNLQVEDSGLYQCVIYQPPKEPHMLFDRIRLVVAAE |
| 11_19_20_ | 119 | MELRAATKLTEEKYELKEGAALDVKCDYTLEKFASSQKAWQIIRDGEMPKTLACTERPSKNSHPV |
| 85 | | QVGRIILEDYHDHGLLRVRMANLQVEDSGLYQCVIYQPPKEPHMLFDRIRLVVTLE |
| 12_22_24_ | 120 | MELRAATKLTEEKYELKEGQTLAVACAYTLEKFASSQKAWQIIRDGEMPKTLACTERPSKNSHPV |
| 26 | | QVGRIILEDYHDHGLLRVRMVNLQVEDSGLYQCVIYQPPKEPHMLFDRIRLVVTLE |
| 13_24_26_ | 121 | MELRAATKLTEEKYELKEGQTLDVACAYALEKFASSQKAWQIIRDGEMPKTLACTERPSKNSHPV |
| 28 | | QVGRIILEDYHDHGLLRVRMVNLQVEDSGLYQCVIYQPPKEPHMLFDRIRLVVTLE |
| 14_26_28_ | 122 | MELRAATKLTEEKYELKEGQTLDVKCAYAAEKFASSQKAWQIIRDGEMPKTLACTERPSKNSHPV |
| 29 | | QVGRIILEDYHDHGLLRVRMVNLQVEDSGLYQCVIYQPPKEPHMLFDRIRLVVTLE |
| 15_28_29_ | 123 | MELRAATKLTEEKYELKEGQTLDVKCDYAAAKFASSQKAWQIIRDGEMPKTLACTERPSKNSHPV |
| 30 | | QVGRIILEDYHDHGLLRVRMVNLQVEDSGLYQCVIYQPPKEPHMLFDRIRLVVTLE |
| 16_29_30_ | 124 | MELRAATKLTEEKYELKEGQTLDVKCDYTAAAFASSQKAWQIIRDGEMPKTLACTERPSKNSHPV |
| 31 | | QVGRIILEDYHDHGLLRVRMVNLQVEDSGLYQCVIYQPPKEPHMLFDRIRLVVTLE |
| 17_31_32_ | 125 | MELRAATKLTEEKYELKEGQTLDVKCDYTLEAAASAQKAWQIIRDGEMPKTLACTERPSKNSHPV |
| 35 | | QVGRIILEDYHDHGLLRVRMVNLQVEDSGLYQCVIYQPPKEPHMLFDRIRLVVTLE |
| 18_30_34_ | 126 | MELRAATKLTEEKYELKEGQTLDVKCDYTLAKFAASQKAWQIIRDGEMPKTLACTERPSKNSHPV |
| | | QVGRIILEDYHDHGLLRVRMVNLQVEDSGLYQCVIYQPPKEPHMLFDRIRLVVTLE |
| 19_34_35_ | 127 | MELRAATKLTEEKYELKEGQTLDVKCDYTLEKFAAAQKAWQIIRDGEMPKTLACTERASKNSHPV |
| 57 | | QVGRIILEDYHDHGLLRVRMVNLQVEDSGLYQCVIYQPPKEPHMLFDRIRLVVTLE |
| 20_35_36_ | 128 | MELRAATKLTEEKYELKEGQTLDVKCDYTLEKFASAAKAWQIIRDGEMPKTLACTEAPSKNSHPV |
| 56 | | QVGRIILEDYHDHGLLRVRMVNLQVEDSGLYQCVIYQPPKEPHMLFDRIRLVVTLE |
| 21_40_49_ | 129 | MELRAATKLTEEKYELKEGQTLDVKCDYTLEKFASSQKAWAIIRDGEMPAALACTERPSKNSHPV |
| 50 | | QVGRIILEDYHDHGLLRVRMVNLQVEDSGLYQCVIYQPPKEPHMLFDRIRLVVTLE |
| 22_43_44_ | 130 | MELRAATKLTEEKYELKEGQTLDVKCDYTLEKFASSQKAWQIIAAGEAPKTLACTERPSKNSHPV |
| 47 | | QVGRIILEDYHDHGLLRVRMVNLQVEDSGLYQCVIYQPPKEPHMLFDRIRLVVTLE |
| 23_43_44_ | 131 | MELRAATKLTEEKYELKEGQTLDVKCDYTLEKFASSQKAWQIIAAAEMPKTLACTERPSKNSHPV |
| 45 | | QVGRIILEDYHDHGLLRVRMVNLQVEDSGLYQCVIYQPPKEPHMLFDRIRLVVTLE |
| 24_44_45_ | 132 | MELRAATKLTEEKYELKEGQTLDVKCDYTLEKFASSQKAWQIIRAAAMPKTLACTERPSKNSHPV |
| 46 | | QVGRIILEDYHDHGLLRVRMVNLQVEDSGLYQCVIYQPPKEPHMLFDRIRLVVTLE |
| 25_45_46_ | 133 | MELRAATKLTEEKYELKEGQTLDVKCDYTLEKFASSQKAWQIIRDAAAPKTLACTERPSKNSHPV |
| 47 | | QVGRIILEDYHDHGLLRVRMVNLQVEDSGLYQCVIYQPPKEPHMLFDRIRLVVTLE |
| 26_43_46_ | 134 | MELRAATKLTEEKYELKEGQTLDVKCDYTLEKFASSQKAWQIIADGAAPKTLACTERPSKNSHPV |
| 47 | | QVGRIILEDYHDHGLLRVRMVNLQVEDSGLYQCVIYQPPKEPHMLFDRIRLVVTLE |
| 27_47_48_ | 135 | MELRAATKLTEEKYELKEGQTLDVKCDYTLEKFASSQKAWQIIRDGEAAATLACTERPSKNSHPV |
| 49 | | QVGRIILEDYHDHGLLRVRMVNLQVEDSGLYQCVIYQPPKEPHMLFDRIRLVVTLE |
| 28_47_49_ | 136 | MELRAATKLTEEKYELKEGQTLDVKCDYTLEKFASSQKAWQIIRDGEAPAALACTERPSKNSHPV |
| 50 | | QVGRIILEDYHDHGLLRVRMVNLQVEDSGLYQCVIYQPPKEPHMLFDRIRLVVTLE |
| 29_49_50_ | 137 | MELRAATKLTEEKYELKEGQTLDVKCDYTLEKFASSQKAWQIIRDGEMPAALAATERPSKNSHPV |
| 53 | | QVGRIILEDYHDHGLLRVRMVNLQVEDSGLYQCVIYQPPKEPHMLFDRIRLVVTLE |
| 30_50_53_ | 138 | MELRAATKLTEEKYELKEGQTLDVKCDYTLEKFASSQKAWQIIRDGEMPKALAATARPSKNSHPV |
| 55 | | QVGRIILEDYHDHGLLRVRMVNLQVEDSGLYQCVIYQPPKEPHMLFDRIRLVVTLE |
| 31_55_56_ | 139 | MELRAATKLTEEKYELKEGQTLDVKCDYTLEKFASSQKAWQIIRDGEMPKTLACTAAASKNSHPV |
| 57 | | QVGRIILEDYHDHGLLRVRMVNLQVEDSGLYQCVIYQPPKEPHMLFDRIRLVVTLE |
| 32_34_56_ | 140 | MELRAATKLTEEKYELKEGQTLDVKCDYTLEKFAASQKAWQIIRDGEMPKTLACTEAASKNSHPV |
| 57 | | QVGRIILEDYHDHGLLRVRMVNLQVEDSGLYQCVIYQPPKEPHMLFDRIRLVVTLE |
| 33_57_59_ | 141 | MELRAATKLTEEKYELKEGQTLDVKCDYTLEKFASSQKAWQIIRDGEMPKTLACTERASAASHPV |
| 60 | | QVGRIILEDYHDHGLLRVRMVNLQVEDSGLYQCVIYQPPKEPHMLFDRIRLVVTLE |
| 34_59_60_ | 142 | MELRAATKLTEEKYELKEGQTLDVKCDYTLEKFASSQKAWQIIRDGEMPKTLACTERPSAAAHPV |
| 61 | | QVGRIILEDYHDHGLLRVRMVNLQVEDSGLYQCVIYQPPKEPHMLFDRIRLVVTLE |
| 35_60_61_ | 143 | MELRAATKLTEEKYELKEGQTLDVKCDYTLEKFASSQKAWQIIRDGEMPKTLACTERPSKAAAPV |
| 62 | | QVGRIILEDYHDHGLLRVRMVNLQVEDSGLYQCVIYQPPKEPHMLFDRIRLVVTLE |
| 36_61_62_ | 144 | MELRAATKLTEEKYELKEGQTLDVKCDYTLEKFASSQKAWQIIRDGEMPKTLACTERPSKNAAAV |
| 63 | | QVGRIILEDYHDHGLLRVRMVNLQVEDSGLYQCVIYQPPKEPHMLFDRIRLVVTLE |
| 37_62_63_ | 145 | MELRAATKLTEEKYELKEGQTLDVKCDYTLEKFASSQKAWQIIRDGEMPKTLACTERPSKNSAAA |
| 64 | | QVGRIILEDYHDHGLLRVRMVNLQVEDSGLYQCVIYQPPKEPHMLFDRIRLVVTLE |
| 38_63_64_ | 146 | MELRAATKLTEEKYELKEGQTLDVKCDYTLEKFASSQKAWQIIRDGEMPKTLACTERPSKNSHAA |
| 65 | | AVGRIILEDYHDHGLLRVRMVNLQVEDSGLYQCVIYQPPKEPHMLFDRIRLVVTLE |
| 39_64_65_ | 147 | MELRAATKLTEEKYELKEGQTLDVKCDYTLEKFASSQKAWQIIRDGEMPKTLACTERPSKNSHPA |
| 66 | | AAGRIILEDYHDHGLLRVRMVNLQVEDSGLYQCVIYQPPKEPHMLFDRIRLVVTLE |
| 40_60_74_ | 148 | MELRAATKLTEEKYELKEGQTLDVKCDYTLEKFASSQKAWQIIRDGEMPKTLACTERPSKASHPV |
| 76 | | QVGRIILEDAHAHGLLRVRMVNLQVEDSGLYQCVIYQPPKEPHMLFDRIRLVVTLE |
| 41_60_75_ | 149 | MELRAATKLTEEKYELKEGQTLDVKCDYTLEKFASSQKAWQIIRDGEMPKTLACTERPSKASHPV |
| 76 | | QVGRIILEDYAAHGLLRVRMVNLQVEDSGLYQCVIYQPPKEPHMLFDRIRLVVTLE |
| 42_60_76_ | 150 | MELRAATKLTEEKYELKEGQTLDVKCDYTLEKFASSQKAWQIIRDGEMPKTLACTERPSKASHPV |
| 77 | | QVGRIILEDYHAAGLLRVRMVNLQVEDSGLYQCVIYQPPKEPHMLFDRIRLVVTLE |
| 43_28_76_ | 151 | MELRAATKLTEEKYELKEGQTLDVKCDYALEKFASSQKAWQIIRDGEMPKTLACTERPSKNSHPV |
| 77 | | QVGRIILEDYHAAGLLRVRMVNLQVEDSGLYQCVIYQPPKEPHMLFDRIRLVVTLE |
| 44_28_77_ | 152 | MELRAATKLTEEKYELKEGQTLDVKCDYALEKFASSQKAWQIIRDGEMPKTLACTERPSKNSHPV |
| 78 | | QVGRIILEDYHDAALLRVRMVNLQVEDSGLYQCVIYQPPKEPHMLFDRIRLVVTLE |
| 45_96_109_ | 153 | MELRAATKLTEEKYELKEGQTLDVKCDYTLEKFASSQKAWQIIRDGEMPKTLACTERPSKNSHPV |
| 111 | | QVGRIILEDYHDHGLLRVRMVNLQVEDSGLYACVIYQPPKEPHMAFARIRLVVTLE |
| 46_98_100_ | 154 | MELRAATKLTEEKYELKEGQTLDVKCDYTLEKFASSQKAWQIIRDGEMPKTLACTERPSKNSHPV |
| 109 | | QVGRIILEDYHDHGLLRVRMVNLQVEDSGLYQCAIAQPPKEPHMAFDRIRLVVTLE |
| 47_35_100_ | 155 | MELRAATKLTEEKYELKEGQTLDVKCDYTLEKFASAQKAWQIIRDGEMPKTLACTERPSKNSHPV |
| 102 | | QVGRIILEDYHDHGLLRVRMVNLQVEDSGLYQCVIAQAPKEPHMLFDRIRLVVTLE |
| 48_102_103_ | 156 | MELRAATKLTEEKYELKEGQTLDVKCDYTLEKFASSQKAWQIIRDGEMPKTLACTERPSKNSHPV |
| 104 | | QVGRIILEDYHDHGLLRVRMVNLQVEDSGLYQCVIYQAAAEPHMLFDRIRLVVTLE |
| 49_103_104_ | 157 | MELRAATKLTEEKYELKEGQTLDVKCDYTLEKFASSQKAWQIIRDGEMPKTLACTERPSKNSHPV |
| 105 | | QVGRIILEDYHDHGLLRVRMVNLQVEDSGLYQCVIYQPAAAPHMLFDRIRLVVTLE |
| 50_104_105_ | 158 | MELRAATKLTEEKYELKEGQTLDVKCDYTLEKFASSQKAWQIIRDGEMPKTLACTERPSKNSHPV |
| 106 | | QVGRIILEDYHDHGLLRVRMVNLQVEDSGLYQCVIYQPPAAAHMLFDRIRLVVTLE |
| 51_105_106_ | 159 | MELRAATKLTEEKYELKEGQTLDVKCDYTLEKFASSQKAWQIIRDGEMPKTLACTERPSKNSHPV |
| 107 | | QVGRIILEDYHDHGLLRVRMVNLQVEDSGLYQCVIYQPPKAAAMLFDRIRLVVTLE |
| 52_106_107_ | 160 | MELRAATKLTEEKYELKEGQTLDVKCDYTLEKFASSQKAWQIIRDGEMPKTLACTERPSKNSHPV |
| 108 | | QVGRIILEDYHDHGLLRVRMVNLQVEDSGLYQCVIYQPPKEAAALFDRIRLVVTLE |
| 53_107_108_ | 161 | MELRAATKLTEEKYELKEGQTLDVKCDYTLEKFASSQKAWQIIRDGEMPKTLACTERPSKNSHPV |
| 109 | | QVGRIILEDYHDHGLLRVRMVNLQVEDSGLYQCVIYQPPKEPAAAFDRIRLVVTLE |
| 54_109_110_ | 162 | MELRAATKLTEEKYELKEGQTLDVKCDYTLEKFASSQKAWQIIRDGEMPKTLACTERPSKNSHPV |
| 111 | | QVGRIILEDYHDHGLLRVRMVNLQVEDSGLYQCVIYQPPKEPHMAAARIRLVVTLE |
| 55_11_113_ | 163 | MELRAATKLTEAKYELKEGQTLDVKCDYTLEKFASSQKAWQIIRDGEMPKTLACTERPSKNSHPV |
| 114 | | QVGRIILEDYHDHGLLRVRMVNLQVEDSGLYQCVIYQPPKEPHMLFDRAALVVTLE |
| 56_10_12_ | 164 | MELRAATKLTAEAYELKEGQTLDVKCDYTLEKFASSQKAWQIIRDGEMPKTLACTERPSKNSHPV |
| 114 | | QVGRIILEDYHDHGLLRVRMVNLQVEDSGLYQCVIYQPPKEPHMLFDRIALVVTLE |
| 57_118_119_ | 165 | MELRAATKLTEEKYELKEGQTLDVKCDYTLEKFASSQKAWQIIRDGEMPKTLACTERPSKNSHPV |
| 120 | | QVGRIILEDYHDHGLLRVRMVNLQVEDSGLYQCVIYQPPKEPHMLFDRIRLVVAAA |
| 58_16_119_ | 166 | MELRAATKLTEEKYELAEGQTLDVKCDYTLEKFASSQKAWQIIRDGEMPKTLACTERPSKNSHPV |
| 120 | | QVGRIILEDYHDHGLLRVRMVNLQVEDSGLYQCVIYQPPKEPHMLFDRIRLVVTAA |

TABLE 9
List of TREM1 protein sequences used to design the two-alanine mutant array

| | SEQ | |
|---|---|---|
| | ID | |
| ID | NO | TREM1 polypeptide sequence |
| 01_WT | 167 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 02_2_3 | 168 | AAAALTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 03_3_4 | 169 | AATAATEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 04_4_5 | 170 | AATKAAEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 05_5_104 | 171 | AATKLAEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHALFDRIRLVVTKG |
| 06_5_106 | 172 | AATKLAEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLADRIRLVVTKG |
| 07_5_107 | 173 | AATKLAEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFARIRLVVTKG |
| 08_7_9 | 174 | AATKLTEAKAELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 09_7_107 | 175 | AATKLTEAKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFARIRLVVTKG |
| 10_8_10 | 176 | AATKLTEEAYALKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 11_11_12 | 177 | AATKLTEEKYEAAEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 12_12_13 | 178 | AATKLTEEKYELAAGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 13_12_15 | 179 | AATKLTEEKYELAEGATLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 14_13_85 | 180 | AATKLTEEKYELKAGQTLDVKCDYTLEKFASSQKAWQIIRDGEMPKTLACTERPSKNSHPVQVGRI |
| | | ILEDYHDHGLLRVRMVNLQAEDSGLYQCVIYQPPKEPHMLFDRIRLVVTKG |
| 15_16_81 | 181 | AATKLTEEKYELKEGQALDVKCDYTLEKFASSQKAWQIIRDGEMPKTLACTERPSKNSHPVQVGRI |
| | | ILEDYHDHGLLRVRMANLQVEDSGLYQCVIYQPPKEPHMLFDRIRLVVTKG |
| 16_20_75 | 182 | AATKLTEEKYELKEGQTLDVACDYTLEKFASSQKAWQIIRDGEMPKTLACTERPSKNSHPVQVGRI |
| | | ILEDYHDHGALRVRMVNLQVEDSGLYQCVIYQPPKEPHMLFDRIRLVVTKG |
| 17_24_25 | 183 | AATKLTEEKYELKEGQTLDVKCDYAAEKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 18_24_26 | 184 | AATKLTEEKYELKEGQTLDVKCDYALAKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 19_24_27 | 185 | AATKLTEEKYELKEGQTLDVKCDYALEAFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 20_25_26 | 186 | AATKLTEEKYELKEGQTLDVKCDYTAAKFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 21_26_27 | 187 | AATKLTEEKYELKEGQTLDVKCDYTLAAFASSQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 22_30_31 | 188 | AATKLTEEKYELKEGQTLDVKCDYTLEKFAAAQKAWQIIRDGEMPKTLACTERPSKNSHPVQVGRI |
| | | ILEDYHDHGLLRVRMVNLQVEDSGLYQCVIYQPPKEPHMLFDRIRLVVTKG |
| 23_30_53 | 189 | AATKLTEEKYELKEGQTLDVKCDYTLEKFAASQKAWQIIRDGEMPKTLACTERASKNSHPVQVGRI |
| | | ILEDYHDHGLLRVRMVNLQVEDSGLYQCVIYQPPKEPHMLFDRIRLVVTKG |
| 24_37_38 | 190 | AATKLTEEKYELKEGQTLDVKCDYTLEKFASSQKAWQAARDGEMPKTLACTERPSKNSHPVQVGRI |
| | | ILEDYHDHGLLRVRMVNLQVEDSGLYQCVIYQPPKEPHMLFDRIRLVVTKG |
| 25_37_39 | 191 | AATKLTEEKYELKEGQTLDVKCDYTLEKFASSQKAWQAIADGEMPKTLACTERPSKNSHPVQVGRI |
| | | ILEDYHDHGLLRVRMVNLQVEDSGLYQCVIYQPPKEPHMLFDRIRLVVTKG |
| 26_37_45 | 192 | AATKLTEEKYELKEGQTLDVKCDYTLEKFASSQKAWQAIRDGEMPATLACTERPSKNSHPVQVGRI |
| | | ILEDYHDHGLLRVRMVNLQVEDSGLYQCVIYQPPKEPHMLFDRIRLVVTKG |
| 27_37_47 | 193 | AATKLTEEKYELKEGQTLDVKCDYTLEKFASSQKAWQAIRDGEMPKTAACTERPSKNSHPVQVGRI |
| | | ILEDYHDHGLLRVRMVNLQVEDSGLYQCVIYQPPKEPHMLFDRIRLVVTKG |
| 28_38_44 | 194 | AATKLTEEKYELKEGQTLDVKCDYTLEKFASSQKAWQIARDGEMAKTLACTERPSKNSHPVQVGRI |
| | | ILEDYHDHGLLRVRMVNLQVEDSGLYQCVIYQPPKEPHMLFDRIRLVVTKG |
| 29_38_90 | 195 | AATKLTEEKYELKEGQTLDVKCDYTLEKFASSQKAWQIARDGEMPKTLACTERPSKNSHPVQVGRI |
| | | ILEDYHDHGLLRVRMVNLQVEDSGAYQCVIYQPPKEPHMLFDRIRLVVTKG |
| 30_42_43 | 196 | AATKLTEEKYELKEGQTLDVKCDYTLEKFASSQKAWQIIRDGAAPKTLACTERPSKNSHPVQVGRI |
| | | ILEDYHDHGLLRVRMVNLQVEDSGLYQCVIYQPPKEPHMLFDRIRLVVTKG |
| 31_42_45 | 197 | AATKLTEEKYELKEGQTLDVKCDYTLEKFASSQKAWQIIRDGAMPATLACTERPSKNSHPVQVGRI |
| | | ILEDYHDHGLLRVRMVNLQVEDSGLYQCVIYQPPKEPHMLFDRIRLVVTKG |
| 32_43_44 | 198 | AATKLTEEKYELKEGQTLDVKCDYTLEKFASSQKAWQIIRDGEAAKTLACTERPSKNSHPVQVGRI |
| | | ILEDYHDHGLLRVRMVNLQVEDSGLYQCVIYQPPKEPHMLFDRIRLVVTKG |
| 33_43_10 | 199 | AATKLTEEKYELKEGQTLDVKCDYTLEKFASSQKAWQIIRDGEAPKTLACTERPSKNSHPVQVGRI |
| | | ILEDYHDHGLLRVRMVNLQVEDSGLYQCVIYQPPKAPHMLFDRIRLVVTKG |
| 34_44_45 | 200 | AATKLTEEKYELKEGQTLDVKCDYTLEKFASSQKAWQIIRDGEMAATLACTERPSKNSHPVQVGRI |
| | | ILEDYHDHGLLRVRMVNLQVEDSGLYQCVIYQPPKEPHMLFDRIRLVVTKG |
| 35_45_47 | 201 | AATKLTEEKYELKEGQTLDVKCDYTLEKFASSQKAWQIIRDGEMPATAACTERPSKNSHPVQVGRI |
| | | ILEDYHDHGLLRVRMVNLQVEDSGLYQCVIYQPPKEPHMLFDRIRLVVTKG |
| 36_47_65 | 202 | AATKLTEEKYELKEGQTLDVKCDYTLEKFASSQKAWQIIRDGEMPKTAACTERPSKNSHPVQVGRA |
| | | ILEDYHDHGLLRVRMVNLQVEDSGLYQCVIYQPPKEPHMLFDRIRLVVTKG |
| 37_51_55 | 203 | AATKLTEEKYELKEGQTLDVKCDYTLEKFASSQKAWQIIRDGEMPKTLACTARPSANSHPVQVGRI |
| | | ILEDYHDHGLLRVRMVNLQVEDSGLYQCVIYQPPKEPHMLFDRIRLVVTKG |
| 38_56_57 | 204 | AATKLTEEKYELKEGQTLDVKCDYTLEKFASSQKAWQIIRDGEMPKTLACTERPSKAAHPVQVGRI |
| | | ILEDYHDHGLLRVRMVNLQVEDSGLYQCVIYQPPKEPHMLFDRIRLVVTKG |
| 39_58_59 | 205 | AATKLTEEKYELKEGQTLDVKCDYTLEKFASSQKAWQIIRDGEMPKTLACTERPSKNSAAVQVGRI |
| | | ILEDYHDHGLLRVRMVNLQVEDSGLYQCVIYQPPKEPHMLFDRIRLVVTKG |
| 40_59_60 | 206 | AATKLTEEKYELKEGQTLDVKCDYTLEKFASSQKAWQIIRDGEMPKTLACTERPSKNSHAAQVGRI |
| | | ILEDYHDHGLLRVRMVNLQVEDSGLYQCVIYQPPKEPHMLFDRIRLVVTKG |
| 41_59_61 | 207 | AATKLTEEKYELKEGQTLDVKCDYTLEKFASSQKAWQIIRDGEMPKTLACTERPSKNSHAVAVGRI |
| | | ILEDYHDHGLLRVRMVNLQVEDSGLYQCVIYQPPKEPHMLFDRIRLVVTKG |
| 42_59_66 | 208 | AATKLTEEKYELKEGQTLDVKCDYTLEKFASSQKAWQIIRDGEMPKTLACTERPSKNSHAVQVGRI |
| | | ALEDYHDHGLLRVRMVNLQVEDSGLYQCVIYQPPKEPHMLFDRIRLVVTKG |
| 43_60_61 | 209 | AATKLTEEKYELKEGQTLDVKCDYTLEKFASSQKAWQIIRDGEMPKTLACTERPSKNSHPAAVGRI |
| | | ILEDYHDHGLLRVRMVNLQVEDSGLYQCVIYQPPKEPHMLFDRIRLVVTKG |
| 44_60_62 | 210 | AATKLTEEKYELKEGQTLDVKCDYTLEKFASSQKAWQIIRDGEMPKTLACTERPSKNSHPAQAGRI |
| | | ILEDYHDHGLLRVRMVNLQVEDSGLYQCVIYQPPKEPHMLFDRIRLVVTKG |
| 45_61_62 | 211 | AATKLTEEKYELKEGQTLDVKCDYTLEKFASSQKAWQIIRDGEMPKTLACTERPSKNSHPVAAGRI |
| | | ILEDYHDHGLLRVRMVNLQVEDSGLYQCVIYQPPKEPHMLFDRIRLVVTKG |
| 46_61_66 | 212 | AATKLTEEKYELKEGQTLDVKCDYTLEKFASSQKAWQIIRDGEMPKTLACTERPSKNSHPVAVGRI |
| | | ALEDYHDHGLLRVRMVNLQVEDSGLYQCVIYQPPKEPHMLFDRIRLVVTKG |
| 47_62_65 | 213 | AATKLTEEKYELKEGQTLDVKCDYTLEKFASSQKAWQIIRDGEMPKTLACTERPSKNSHPVQAGRA |
| | | ILEDYHDHGLLRVRMVNLQVEDSGLYQCVIYQPPKEPHMLFDRIRLVVTKG |
| 48_65_66 | 214 | AATKLTEEKYELKEGQTLDVKCDYTLEKFASSQKAWQIIRDGEMPKTLACTERPSKNSHPVQVGRA |
| | | ALEDYHDHGLLRVRMVNLQVEDSGLYQCVIYQPPKEPHMLFDRIRLVVTKG |
| 49_66_81 | 215 | AATKLTEEKYELKEGQTLDVKCDYTLEKFASSQKAWQIIRDGEMPKTLACTERPSKNSHPVQVGRI |
| | | ALEDYHDHGLLRVRMANLQVEDSGLYQCVIYQPPKEPHMLFDRIRLVVTKG |
| 50_72_73 | 216 | AATKLTEEKYELKEGQTLDVKCDYTLEKFASSQKAWQIIRDGEMPKTLACTERPSKNSHPVQVGRI |
| | | ILEDYHAAGLLRVRMVNLQVEDSGLYQCVIYQPPKEPHMLFDRIRLVVTKG |
| 51_73_75 | 217 | AATKLTEEKYELKEGQTLDVKCDYTLEKFASSQKAWQIIRDGEMPKTLACTERPSKNSHPVQVGRI |
| | | ILEDYHDAGALRVRMVNLQVEDSGLYQCVIYQPPKEPHMLFDRIRLVVTKG |
| 52_81_82 | 218 | AATKLTEEKYELKEGQTLDVKCDYTLEKFASSQKAWQIIRDGEMPKTLACTERPSKNSHPVQVGRI |
| | | ILEDYHDHGLLRVRMAALQVEDSGLYQCVIYQPPKEPHMLFDRIRLVVTKG |
| 53_85_86 | 219 | AATKLTEEKYELKEGQTLDVKCDYTLEKFASSQKAWQIIRDGEMPKTLACTERPSKNSHPVQVGRI |
| | | ILEDYHDHGLLRVRMVNLQAADSGLYQCVIYQPPKEPHMLFDRIRLVVTKG |
| 54_98_99 | 220 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQAAKEPHMLFDRIRLVVTKG |
| 55_99_100 | 221 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPAAEPHMLFDRIRLVVTKG |
| 56_100_101 | 222 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPAAPHMLFDRIRLVVTKG |
| 57_101_102 | 223 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKAAHMLFDRIRLVVTKG |
| 58_102_103 | 224 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEAAMLFDRIRLVVTKG |
| 59_103_104 | 225 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPAALFDRIRLVVTKG |
| 60_104_106 | 226 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHALADRIRLVVTKG |
| 61_106_107 | 227 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLAARIRLVVTKG |
| 62_8_10_ | 228 | AATKLTEEAYALKEGQTLDVKCDYTLEKFASSQKAWQIIRDGEMPKTLACTERPSKNSHPVQVGRI |
| 112 | | ILEDYHDHGLLRVRMVNLQVEDSGLYQCVIYQPPKEPHMLFDRIRLAVTKG |
| 63_37_39_ | 229 | AATKLTEEKYELKEGQTLDVKCDYTLEKFASSQKAWQAIAAGEMPKTLACTERPSKNSHPVQVGRI |
| 40 | | ILEDYHDHGLLRVRMVNLQVEDSGLYQCVIYQPPKEPHMLFDRIRLVVTKG |
| 64_40 | 230 | AATKLTEEKYELKEGQTLDVKCDYTLEKFASSQKAWQIIRAGEMPKTLACTERPSKNSHPVQVGRI |
| | | ILEDYHDHGLLRVRMVNLQVEDSGLYQCVIYQPPKEPHMLFDRIRLVVTKG |
| 112 | 231 | AATKLTEEKYELKEGQTLDVKCDYTLEKFASSQKAWQIIRDGEMPKTLACTERPSKNSHPVQVGRI |
| | | ILEDYHDHGLLRVRMVNLQVEDSGLYQCVIYQPPKEPHMLFDRIRLAVTKG |

TABLE 10
List of TREM1 protein sequences used to design the single-alanine mutant array

| | SEQ | |
|---|---|---|
| | ID | |
| ID | NO | TREM1 polypeptide sequence |
| 01_WT | 232 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 02_1 | 233 | AAKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 03_2 | 234 | ATALTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 04_4 | 235 | ATKLAEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 05_5 | 236 | ATKLTAEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 06_6 | 237 | ATKLTEAKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 07_7 | 238 | ATKLTEEAYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 08_8 | 239 | ATKLTEEKAELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 09_9 | 240 | ATKLTEEKYALKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 10_11 | 241 | ATKLTEEKYELAEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 11_14 | 242 | ATKLTEEKYELKEGATLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 12_17 | 243 | ATKLTEEKYELKEGQTLAVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 13_19 | 244 | ATKLTEEKYELKEGQTLDVACDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 14_21 | 245 | ATKLTEEKYELKEGQTLDVKCAYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 15_23 | 246 | ATKLTEEKYELKEGQTLDVKCDYALEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 16_24 | 247 | ATKLTEEKYELKEGQTLDVKCDYTAEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 17_25 | 248 | ATKLTEEKYELKEGQTLDVKCDYTLAKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 18_26 | 249 | ATKLTEEKYELKEGQTLDVKCDYTLEAFASSQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 19_29 | 250 | ATKLTEEKYELKEGQTLDVKCDYTLEKFAASQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 20_30 | 251 | ATKLTEEKYELKEGQTLDVKCDYTLEKFASAQKAWQIIRDGEMPKTLACTERPSKNSHPVQVGRII |
| | | LEDYHDHGLLRVRMVNLQVEDSGLYQCVIYQPPKEPHMLFDRIRLVVTLE |
| 21_38 | 252 | ATKLTEEKYELKEGQTLDVKCDYTLEKFASSQKAWQIIADGEMPKTLACTERPSKNSHPVQVGRII |
| | | LEDYHDHGLLRVRMVNLQVEDSGLYQCVIYQPPKEPHMLFDRIRLVVTLE |
| 22_39 | 253 | ATKLTEEKYELKEGQTLDVKCDYTLEKFASSQKAWQIIRAGEMPKTLACTERPSKNSHPVQVGRII |
| | | LEDYHDHGLLRVRMVNLQVEDSGLYQCVIYQPPKEPHMLFDRIRLVVTLE |
| 23_41 | 254 | ATKLTEEKYELKEGQTLDVKCDYTLEKFASSQKAWQIIRDGAMPKTLACTERPSKNSHPVQVGRII |
| | | LEDYHDHGLLRVRMVNLQVEDSGLYQCVIYQPPKEPHMLFDRIRLVVTLE |
| 24_42 | 255 | ATKLTEEKYELKEGQTLDVKCDYTLEKFASSQKAWQIIRDGEAPKTLACTERPSKNSHPVQVGRII |
| | | LEDYHDHGLLRVRMVNLQVEDSGLYQCVIYQPPKEPHMLFDRIRLVVTLE |
| 25_44 | 256 | ATKLTEEKYELKEGQTLDVKCDYTLEKFASSQKAWQIIRDGEMPATLACTERPSKNSHPVQVGRII |
| | | LEDYHDHGLLRVRMVNLQVEDSGLYQCVIYQPPKEPHMLFDRIRLVVTLE |
| 26_45 | 257 | ATKLTEEKYELKEGQTLDVKCDYTLEKFASSQKAWQIIRDGEMPKALACTERPSKNSHPVQVGRII |
| | | LEDYHDHGLLRVRMVNLQVEDSGLYQCVIYQPPKEPHMLFDRIRLVVTLE |
| 27_50 | 258 | ATKLTEEKYELKEGQTLDVKCDYTLEKFASSQKAWQIIRDGEMPKTLACTARPSKNSHPVQVGRII |
| | | LEDYHDHGLLRVRMVNLQVEDSGLYQCVIYQPPKEPHMLFDRIRLVVTLE |
| 28_51 | 259 | ATKLTEEKYELKEGQTLDVKCDYTLEKFASSQKAWQIIRDGEMPKTLACTEAPSKNSHPVQVGRII |
| | | LEDYHDHGLLRVRMVNLQVEDSGLYQCVIYQPPKEPHMLFDRIRLVVTLE |
| 29_52 | 260 | ATKLTEEKYELKEGQTLDVKCDYTLEKFASSQKAWQIIRDGEMPKTLACTERASKNSHPVQVGRII |
| | | LEDYHDHGLLRVRMVNLQVEDSGLYQCVIYQPPKEPHMLFDRIRLVVTLE |
| 30_54 | 261 | ATKLTEEKYELKEGQTLDVKCDYTLEKFASSQKAWQIIRDGEMPKTLACTERPSANSHPVQVGRII |
| | | LEDYHDHGLLRVRMVNLQVEDSGLYQCVIYQPPKEPHMLFDRIRLVVTLE |
| 31_55 | 262 | ATKLTEEKYELKEGQTLDVKCDYTLEKFASSQKAWQIIRDGEMPKTLACTERPSKASHPVQVGRII |
| | | LEDYHDHGLLRVRMVNLQVEDSGLYQCVIYQPPKEPHMLFDRIRLVVTLE |
| 32_56 | 263 | ATKLTEEKYELKEGQTLDVKCDYTLEKFASSQKAWQIIRDGEMPKTLACTERPSKNAHPVQVGRII |
| | | LEDYHDHGLLRVRMVNLQVEDSGLYQCVIYQPPKEPHMLFDRIRLVVTLE |
| 33_57 | 264 | ATKLTEEKYELKEGQTLDVKCDYTLEKFASSQKAWQIIRDGEMPKTLACTERPSKNSAPVQVGRII |
| | | LEDYHDHGLLRVRMVNLQVEDSGLYQCVIYQPPKEPHMLFDRIRLVVTLE |
| 34_58 | 265 | ATKLTEEKYELKEGQTLDVKCDYTLEKFASSQKAWQIIRDGEMPKTLACTERPSKNSHAVQVGRII |
| | | LEDYHDHGLLRVRMVNLQVEDSGLYQCVIYQPPKEPHMLFDRIRLVVTLE |
| 35_59 | 266 | ATKLTEEKYELKEGQTLDVKCDYTLEKFASSQKAWQIIRDGEMPKTLACTERPSKNSHPAQVGRII |
| | | LEDYHDHGLLRVRMVNLQVEDSGLYQCVIYQPPKEPHMLFDRIRLVVTLE |
| 36_60 | 267 | ATKLTEEKYELKEGQTLDVKCDYTLEKFASSQKAWQIIRDGEMPKTLACTERPSKNSHPVAVGRII |
| | | LEDYHDHGLLRVRMVNLQVEDSGLYQCVIYQPPKEPHMLFDRIRLVVTLE |
| 37_61 | 268 | ATKLTEEKYELKEGQTLDVKCDYTLEKFASSQKAWQIIRDGEMPKTLACTERPSKNSHPVQAGRII |
| | | LEDYHDHGLLRVRMVNLQVEDSGLYQCVIYQPPKEPHMLFDRIRLVVTLE |
| 38_63 | 269 | ATKLTEEKYELKEGQTLDVKCDYTLEKFASSQKAWQIIRDGEMPKTLACTERPSKNSHPVQVGAII |
| | | LEDYHDHGLLRVRMVNLQVEDSGLYQCVIYQPPKEPHMLFDRIRLVVTLE |
| 39_65 | 270 | ATKLTEEKYELKEGQTLDVKCDYTLEKFASSQKAWQIIRDGEMPKTLACTERPSKNSHPVQVGRIA |
| | | LEDYHDHGLLRVRMVNLQVEDSGLYQCVIYQPPKEPHMLFDRIRLVVTLE |
| 40_71 | 271 | ATKLTEEKYELKEGQTLDVKCDYTLEKFASSQKAWQIIRDGEMPKTLACTERPSKNSHPVQVGRII |
| | | LEDYHAHGLLRVRMVNLQVEDSGLYQCVIYQPPKEPHMLFDRIRLVVTLE |
| 41_72 | 272 | ATKLTEEKYELKEGQTLDVKCDYTLEKFASSQKAWQIIRDGEMPKTLACTERPSKNSHPVQVGRII |
| | | LEDYHDAGLLRVRMVNLQVEDSGLYQCVIYQPPKEPHMLFDRIRLVVTLE |
| 42_78 | 273 | ATKLTEEKYELKEGQTLDVKCDYTLEKFASSQKAWQIIRDGEMPKTLACTERPSKNSHPVQVGRII |
| | | LEDYHDHGLLRVAMVNLQVEDSGLYQCVIYQPPKEPHMLFDRIRLVVTLE |
| 43_80 | 274 | ATKLTEEKYELKEGQTLDVKCDYTLEKFASSQKAWQIIRDGEMPKTLACTERPSKNSHPVQVGRII |
| | | LEDYHDHGLLRVRMANLQVEDSGLYQCVIYQPPKEPHMLFDRIRLVVTLE |
| 44_81 | 275 | ATKLTEEKYELKEGQTLDVKCDYTLEKFASSQKAWQIIRDGEMPKTLACTERPSKNSHPVQVGRII |
| | | LEDYHDHGLLRVRMVALQVEDSGLYQCVIYQPPKEPHMLFDRIRLVVTLE |
| 45_83 | 276 | ATKLTEEKYELKEGQTLDVKCDYTLEKFASSQKAWQIIRDGEMPKTLACTERPSKNSHPVQVGRII |
| | | LEDYHDHGLLRVRMVNLAVEDSGLYQCVIYQPPKEPHMLFDRIRLVVTLE |
| 46_84 | 277 | ATKLTEEKYELKEGQTLDVKCDYTLEKFASSQKAWQIIRDGEMPKTLACTERPSKNSHPVQVGRII |
| | | LEDYHDHGLLRVRMVNLQAEDSGLYQCVIYQPPKEPHMLFDRIRLVVTLE |
| 47_85 | 278 | ATKLTEEKYELKEGQTLDVKCDYTLEKFASSQKAWQIIRDGEMPKTLACTERPSKNSHPVQVGRII |
| | | LEDYHDHGLLRVRMVNLQVADSGLYQCVIYQPPKEPHMLFDRIRLVVTLE |
| 48_87 | 279 | ATKLTEEKYELKEGQTLDVKCDYTLEKFASSQKAWQIIRDGEMPKTLACTERPSKNSHPVQVGRII |
| | | LEDYHDHGLLRVRMVNLQVEDAGLYQCVIYQPPKEPHMLFDRIRLVVTLE |
| 49_95 | 280 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIAQPPKEPHMLFDRIRLVVTLE |
| 50_97 | 281 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQAPKEPHMLFDRIRLVVTLE |
| 51_98 | 282 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPAKEPHMLFDRIRLVVTLE |
| 52_99 | 283 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPAEPHMLFDRIRLVVTLE |
| 53_100 | 284 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKAPHMLFDRIRLVVTLE |
| 54_101 | 285 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEAHMLFDRIRLVVTLE |
| 55_102 | 286 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPAMLFDRIRLVVTLE |
| 56_103 | 287 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHALFDRIRLVVTLE |
| 57_104 | 288 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMAFDRIRLVVTLE |
| 58_106 | 289 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFARIRLVVTLE |
| 59_109 | 290 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIALVVTLE |
| 60_111 | 291 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLAVTLE |
| 61_113 | 292 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVALE |
| 62_114 | 293 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTAE |
| 63_115 | 294 | ATKLTEEKYELKEGQTLDVKCDYTLEKFASSQKAWQIIRDGEMPKTLACTERPSKNSHPVQVGRII |
| | | LEDYHDHGLLRVRMVNLQVEDSGLYQCVIYQPPKEPHMLFDRIRLVVTLA |

Each of the above clones were expressed as fusion proteins consisting of the TREM1 IgV domain followed by a triple alanine linker fused to a human Fc domain. Each clone was captured onto a sensor coated with an anti-human Fc antibody. The sensors were subsequently dipped into a solution containing an antibody of interest. Binding kinetics were monitored using a Bio-Layer Interferometry (BLI) instrument (Octet RED384 or Octet HTX, ForteBio).

By monitoring the binding kinetics of the antibody to each mutant TREM1 clone and comparing them to the kinetics against the wild type protein, the epitope could be deduced. An increase in the antibody dissociation rate constant or loss of antibody binding to the protein indicated that the mutated residues in that clone were important for antibody binding, and hence part of its epitope.

12172 antibody was selected as a potent inhibitor of TREM1 activity. Interestingly, the alanine scanning approach demonstrated that this molecule possessed an epitope distant to the identified PGLYRP1 ligand binding site. This was subsequently selected as the lead molecule.

Using the above method, and taking into consideration all three arrays, the key epitope residues of 12172 antibody were determined to be residues E26, E27, K28, Y29, E30, L31, K32 and Q35 (where the numbering is according to SEQ ID NO: 1).

### Example 3: Antibody Humanization

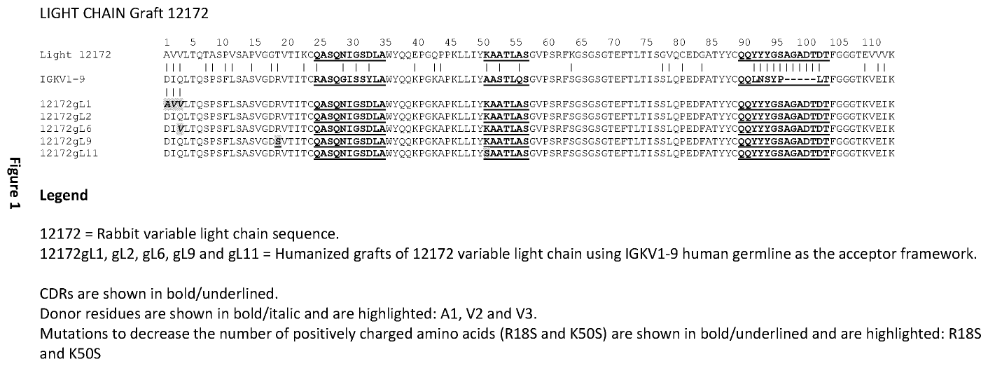

Antibody 12172 was humanized by grafting the CDRs from the rabbit V-region onto human germline antibody V-region frameworks. In order to recover the activity of the antibody, a number of framework residues from the rabbit V-region were also retained in the humanized sequence. These residues were selected using the protocol outlined by Adair et al. (1991) (WO91/09967). Alignments of the rabbit antibody (donor) V-region sequences with the human germline (acceptor) V-region sequences are shown in FIGS. 1 and 2, together with the designed humanized sequences. The CDRs grafted from the donor to the acceptor sequence are as defined by Kabat (Kabat et al., 1987), with the exception of CDR-H1 where the combined Chothia/Kabat definition is used (see Adair et al., WO91/09967).

Human V-region IGKV1-9 plus IGKJ4 J-region (IMGT, http://www.imgt.org/) was chosen as the acceptor for antibody 12172 light chain CDRs. The light chain framework residues in the humanized graft variants are all from the human germline gene, with the exception of none, one, two or three residues from the group comprising residues 1, 2 and 3 (with reference to SEQ ID NO:25), where the donor residues Alanine (A1), Valine (V2) and Valine (V3) were retained, respectively (FIG. 1 and Table 11).

Human V-region IGHV3-66 plus IGHJ4 J-region (IMGT, http://www.imgt.org/) was chosen as the acceptor for the heavy chain CDRs of antibody 12172. In common with many rabbit antibodies, the VH gene of antibody 12172 is shorter than the selected human acceptor. When aligned with the human acceptor sequence, framework 1 of the VH region of antibody 12172 lacks the N-terminal residue, which is retained in the humanized antibody (FIG. 2). Framework 3 of the 12172 rabbit VH region also lacks two residues (75 and 76, with reference to SEQ ID NO:45) in the loop between beta sheet strands D and E: in the humanized graft variants the gap is filled with the corresponding residues (Lysine 75, K75; Asparagine 76, N76) from the selected human acceptor sequence (FIG. 2). The heavy chain framework residues in the humanized graft variants are all from the human germline gene, with the exception of one or more residues from the group comprising residues 23, 48, 49, 71, 73 and 78 (with reference to SEQ ID NO: 45), where the donor residues Threonine (T23), Isoleucine (I48), Glycine (G49), Lysine (K71), Serine (S73) and Valine (V78) were retained, respectively.

Genes encoding a number of variant heavy and light chain V-region sequences were designed and constructed by an automated synthesis approach by ATUM (CA, USA). Further variants of heavy and light chain V-regions were created by modifying the VH and VK genes by oligonucleotide-directed mutagenesis. For transient expression in mammalian cells, the humanized light chain V-region genes were cloned into the UCB light chain expression vector pMhCK, which contains DNA encoding the human Kappa chain constant region (Km3 allotype). The humanized heavy chain V-region genes were cloned into the UCB human gamma-4 heavy chain expression vector pMhγ4PFL, which contains DNA encoding the human gamma-4 heavy chain constant region with the hinge stabilising mutation S228P (Angal S., King D. J., Bodmer M. W., Turner A., Lawson A. D. G., Roberts G., Pedley B. and Adair J. R. A single amino acid substitution abolishes the heterogeneity of chimeric mouse/human (IgG4) antibody. Mol. Immunol. 1993, 30 (1):105-8), or into the UCB gamma-1 LALA heavy chain expression vector pMhγ1 L234A L235A, which contains DNA encoding the human gamma-1 heavy chain constant region with mutations L234A and L235A to reduce binding to Fc gamma receptors (FcγR) (Canfield S. M. and Morrison S. L. The Binding affinity of Human IgG for its High Affinity Fc Receptor Is Determined by Multiple Amino Acids in the CH2 Domain and Is Modulated by the Hinge Region. J. Exp. Med. 1991, 173:1483-1491). Co-transfection of the resulting heavy and light chain vectors into Expi293 ™ suspension cells was achieved using ExpiFectamine™ 293 transfection reagent (A14525, ThermoFisher Scientific), and gave expression of the humanized, recombinant IgG4P and IgG1 LALA antibodies.

The variant humanized antibody chains, and combinations thereof, were expressed and assessed for their binding affinity for human TREM1 relative to the parent antibody, their thermal stability by fluorescence based thermal shift assay (as described in Example 13) and propensity to self-interact by AC-SINS (Affinity Capture Self-Interaction Nanoparticle Spectroscopy, as described in Example 17). Retention of VH framework donor residues 148, G49 and K71 in graft gH1 1 was essential for the highest affinity binding to human TREM1, as measured by surface plasmon resonance (Table 11). The light chain framework residues in graft gL2 were all from the human germline gene. Retention of VL donor residue V3 in graft gL6 reduced the propensity for self-interaction as measured by AC-SINS assay (Table 22).

Resistance to thermal unfolding (denaturation) is an indicator of conformational stability and long-term storage stability. The humanized IgG4P antibodies have good thermal stability, with the midpoint of unfolding (Tm) for the Fab domains in the range of 73.5-74.6° C. (Table 11).

Molecular self-interaction can lead to native state aggregation and poor solubility, particularly at high protein concentrations used for the sub-cutaneous administration of therapeutic mAbs. The net charge of an antibody Fv domain has been shown to influence native state aggregation of human IgGs at pH 7.4 and pH 5.0 in an isotype specific manner (Heads J T, Lamb R, Kelm S, Adams R, Elliott P, Tyson K, Topia S, West S, Nan R, Turner A, Lawson A D G. Electrostatic interactions modulate the differential aggregation propensities of IgG1 and IgG4P antibodies and inform charged residue substitutions for improved developability. Protein Eng Des Sel. 2019 Dec. 31; 32(6):277-288. doi: 10.1093/protein/gzz046. PMID: 31868219; PMCID: PMC7036597). In order to reduce the propensity for self-interaction of humanized 12172 hIgG4P antibodies, as indicated by a high Δλmax value measured by AC-SINS assay (Table 12 in this Example and Table 22 in Example 17), the net positive charge of the Fv domain was decreased by the mutation of positively charged residues to either neutral or negatively charged residues. These residues were selected using the rationale outlined by Heads et al (2019) (WO2019/234094). Residues 18 and 50 of the humanized light chain graft 12712gL2 (SEQ ID NO: 29) were mutated from Arginine (R18) to Serine (S18) and Lysine (K50) to Serine (S50) in grafts gL9 and gL11, respectively. Residue 75 in the humanized heavy chain graft 12172gH1 1 (SEQ ID NO: 79) was mutated from Lysine (K75) to either Serine (S75), Glutamine (Q75) or Glutamic acid (E75) in grafts gH26, gH48 and gH49, respectively. The modified heavy and light chain genes were transiently expressed in Expi293™ suspension cells in combination, and the recombinant IgG4P antibodies assessed for their binding affinity to human TREM1, thermal stability and propensity to self-interact (Table 12). The humanized 12172 charge mutants retained affinity to human TREM1, and demonstrated a decreased propensity for self-interaction as indicated by a decrease in the AXmax measured by AC-SINS assay.

Biophysical characterization of humanized 12172 gL2gH11 and 12172 gL6gH6, (both hIgG4P and hIgG1 LALA formats) was performed using different stress conditions to assess developability as described in examples 12-20. Additionally, all molecules were analysed by liquid chromatography mass spectrometry (LC-MS) to confirm that the predicted sequence molecular weight (MW) was consistent with experimental data.

The humanized 12172gL2gH11 IgG4P antibody showed similar inhibition of NF-κB in the THP1 monocyte TREM1/DAP12 NF-κB Luciferase reporter cell assay (described in Example 2) to the rabbit parental 12172 antibody (see Table 13).

TABLE 11
Affinity and Tm of various 12172 antibody variants.

| | Light | | | |
|---|---|---|---|---|
| | chain | | Affinity | |
| Antibody | Donor | Heavy chain Donor | (KD) | Tm |
| 12172 variant | residues | residues | pM | (deg. C.) |
| 12172 | — | — | 519/461 | |
| 12172gL1gH1 | A1, V2, | T23, I48, G49, K71, | 437 | 73.5 |
| | V3 | S73, V78 | | |
| 12172gL2gH1 | — | T23, I48, G49, K71, | 442 | 74.1 |
| | | S73, V78 | | |
| 12172gL2gH4 | — | T23, I48, G49, S73, V78 | 693 | 74.3 |
| 12172gL2gH6 | — | T23, I48, G49, K71, S73 | 581 | 73.9 |
| 12172gL2gH8 | — | I48, G49, K71, S73, V78 | 476 | 74.2 |
| 12172gL6gH4 | V3 | T23, I48, G49, S73, V78 | 619 | 74.1 |
| 12172gL6gH6 | V3 | T23, I48, G49, K71, S73 | 463 | 74.5 |
| 12172gL6gH11 | V3 | I48, G49, K71 | 336 | 74.5 |
| 12172gL2gH2 | — | G49, K71, S73, V78 | 989 | 73.6 |
| 12172gL2gH9 | — | I48, G49, V78 | 362 | 74.6 |
| 12172gL2gH10 | — | I48, G49, S73 | 1970 | 74.1 |
| 12172gL2gH11 | — | I48, G49, K71 | 453 | 74.4 |
| 12172gL2gH12 | — | I48, G49 | 718 | 74.1 |

TABLE 12
Affinity, Tm and AC-SINS of various 12172 antibody variants
(replicate values are provided for some of the variants)

| Antibody | Light chain | Light chain | Heavy chain | Heavy chain | Affinity | Fab | |
|---|---|---|---|---|---|---|---|
| 12172 variant | Donor residues | Mutation | Donor residues | Mutation | (KD) pM | Tm | AC-SINS |
| 12172 | — | — | — | — | 570 | | |
| 12172gL2gH11 | — | — | I48, G49, K71 | — | 445 | 74.4 | 10.2 |
| 12172gL2gH26 | — | — | I48, G49, K71 | K75S | 624/672 | 74.1 | 1.98/1.17 |
| 12172gL9gH11 | — | R18S | I48, G49, K71 | — | 550 | ND | 9.38 |
| 12172gL11gH11 | — | K50S | I48, G49, K71 | — | 550 | 73.8 | 7.02/10.56 |
| 12172gL9gH26 | — | R18S | I48, G49, K71 | K75S | 559/616 | 73.8 | 1.73/0.68 |
| 12172gL10gH26 | — | K42Q | I48, G49, K71 | K75S | 578/659 | 73.8 | 2.06/1.33 |
| 12172gL11gH26 | — | K50S | I48, G49, K71 | K75S | 532/633 | 74 | 2.03/1.36 |
| 12172gL9gH48 | — | R18S | I48, G49, K71 | K75Q | 370 | 73.6 | 10.46 |
| 12172gL9gH49 | — | R18S | I48, G49, K71 | K75E | 394 | 73.6 | 6.45 |

TABLE 13
Summary of potency, efficacy and hill slope values for 12172gL2gH11
IgG4P and 12172 Rabbit IgG1

| | Geomean | Average | | |
|---|---|---|---|---|
| | IC50 | Emax | Average | |
| Antibody | (pM) ± SD | (%) ± SD | Slope ± SD | N* |
| 12172gL2gH11 IgG4P | 64 ± 36 | 72 ± 4 | 2.1 ± 0.9 | 3 |
| 12172 Rabbit IgG1 | 84 ± 40 | 89 ± 6 | 2.7 ± 1.2 | 3 |

*Values for each molecule were calculated from three independent experiments.

### Example 4. Expression and Purification of Rabbit 12172 Fab

Co-transfection of heavy and light chain vectors CID 102769 and CID 102770 into Exp1293™ suspension cells was achieved using ExpiFectaminer™ 293 transfection reagent (A 14525, ThermoFisher Scientific), and gave expression of the rabbit recombinant 12172 Fab. The media was filtered through a PALL 0.4/0.2 μm capsule filter and applied to Protein G GammaBind Plus resin (7 mL) settled in XK16/60 column and washed with 10CV 1× PBS pH 7.4. The bound Fab complex proteins were eluted, with 50 mL 100 mM glycine, pH 2.7 and immediately neutralized with 10% 1M Tris, pH 8. Fractions containing 12172 Fab complex were pooled and concentrated in a Vivaspin PES 20, 10 kDa MWCO concentrator. A superdex s200 16/60 column (GE Healthcare) was then used to polish and buffer exchange into 1× PBS pH 7.4. The final protein concentration and purity was assessed as previously described in Example 1.

### Example 5. Expression and Purification of 12172 gL2gH11/gL6gH6 hIgG4P and hIgG1 LALA

### Transient Mammalian Expression, CHOSXE Cultivation

Suspension CHOSXE cells were pre-adapted to CDCHO serum-free media (Invitrogen) supplemented with 2 mM (100×) Glutamax.

Cells were maintained in their logarithmic growth phase, agitated at 190 rpm in a shaking incubator (Kuhner A G, Birsfelden, Switzerland) and cultured at 37° C. supplemented with 8% CO₂.

### Electroporation Transfection

Prior to transfection, cell numbers and viability were determined using a Vi-Cell™ XR Cell Viability Analyser (Beckman Coulter) and the required number of cells (2.3×10⁸ cells/ml) at 99% viability were transferred into centrifuge conical tubes and spun at 1500 rpm for 15 minutes. The pelleted cells were washed in Hyclone™ MaxCyte® buffer (Thermo Scientific) and centrifuged for a further 15 minutes. Pellets were resuspended at 2.3×10⁸ cells/ml in fresh buffer.

Plasmid DNA, purified using a Qiagen Plasmid Plus Giga Kit®, was added at 400 μg/ml. Following electroporation using a MaxCyte STx® flow electroporation instrument, the cells were transferred to ProCHO™ 5 Protein-free CHO medium (Lonza) containing 2 mM Glutamax, 0.75 mM Sodium Butyrate (n-Butyric Acid Sodium Salt, Sigma B-5887), antibiotic antimitotic 100× solutions (1 in 500) and a bolus feed added at day 0.

Transfected cells were then transferred directly into vented flasks and cultured in a Kuhner Shaker Incubator set at 37° C., 8% CO₂ and 190 rpm shaking. Temperature was dropped to 32° C. 24 hrs post transfection and cells were cultured for a further 11-13 days.

On day 12-14, cultures were transferred to centrifuge tubes and supernatant separated from cells after spinning for 30 minutes at 4000 rpm. Retained supernatants were further clarified by filtering through a 0.22 μm Sartobran® P Millipore cartridge, followed by 0.22 μm Millipak® Gamma Gold filters. Final expression titres were determined by Protein G Quantification HPLC assay using A33 hIgG1 at 1 mg/ml as the standard, a 0.8 ml POROS™ G 20 μm column and an Agilent 1100 Series HPLC System. The clarified cell culture harvest was stored at 4° C. prior to purification.

### Antibody Purification and Analysis

Clarified cell culture harvest was allowed to warm to room temperature before loading onto a 215 ml MabSelect™ SuRe™ column (Cytiva) pre-equilibrated into Hyclone™ Phosphate Buffered Saline (PBS) pH7.4, using an ÅKTA Pure 25L chromatography system (Cytiva). After washing in PBS pH7.4, bound material was eluted by reducing the pH to 3.4 (0.1M Sodium Citrate buffer) for human IgG1 isotypes or pH3.7 (30 mM Sodium Acetate) followed by pH3.6 (0.1M Sodium Citrate buffer) for human IgG4P isotypes. Post elution, the column was stripped with 0.1M Citric Acid pH2.0 to remove any strongly bound aggregates. Affinity capture elution peak fractions were pooled and neutralized to pH5.5-7.5 by the addition of 2M Tris-HCl pH8.5. Protein concentration was determined by reading absorbance at 280 nm using a nanodrop and purity was determined by analytical size exclusion HPLC (method below).

Affinity pools were concentrated, using centrifugal filtration devices (Centricon® Plus-70 or Amicon® Ultra-15) or pressurized stirred cell chambers (Amicon®) with a 10KDa or 30KDa MWCO membrane depending on volume, for loading onto a HiLoad Superdex 200 26/60 (Cytiva) or 50/60 prep. grade column (custom packed by Cytiva). The HiLoad Superdex 200 26/60 or 50/60 column was equilibrated into 50 mM Sodium Acetate, 125 mM Sodium Chloride buffer pH5.0 prior to sample loading using an ÅKTA chromatography system (Cytiva). An isocratic elution was run, and fractions were collected after 0.3 CV's. Fractions containing monomer were identified by running fractions or mock pools on analytical size exclusion HPLC. Fractions were pooled to obtain >98% monomer content. Pools were concentrated to 10-15 mg/ml, using centrifugal filtration devices (Centricon® Plus-70 or Amicon® Ultra-15) or pressurized stirred cell chambers (Amicon®) with a 10KDa or 30KDa MWCO membrane depending on volume, then recovered and 0.22 μm sterile filtered using Stericup® filtration units or Millex GV syringe filters.

Final protein concentration was determined by reading absorbance at 280 nm using a nanodrop. Monomer content was determined by analytical size exclusion HPLC. Correct banding pattern was determined by SDS-PAGE using the Invitrogen Novex™ WedgeWell™ 4-20% Tris-Glycine and XCell SureLock™ Mini-Cell Electrophoresis system and Coomassie stain. Endotoxin level was determined using the Charles River Endosafe® LAL Reagent Cartridge Technology and Endosafe® nexgen-PTS reader, with a level of <1EU/mg being of acceptable quality. Samples were analyzed by intact mass spectrometry to confirm heavy and light chain masses, expected modifications and identity.

### Analytical Size Exclusion HPLC

TSKgel G3000SWXL HPLC column (Tosoh) was equilibrated into Hyclone Phosphate Buffered Saline (PBS) pH7.4 using an Agilent 1100 or 1200 series HPLC. 20-50 μg of sample was injected and run in isocratic elution conditions (PBS pH7.4) at 1 ml/min for 16 minutes. Data was compared to BioRad Molecular Weight marker standards. Retention times and percentages were reported for monomer and high and low molecular weight product related impurities.

### Example 6. Binding Kinetics of 12172gL2gH11 hIgG4P to Human and Cynomolgus TREM1

The kinetics of 12172gL2gH11 hIgG4P binding to human and cynomolgus TREM1 were measured at 25° C. by surface plasmon resonance on a Biacore T200 instrument and a Biacore 8k instrument.

A goat anti human IgG, Fc fragment specific antibody (F(ab′)2 fragment, Jackson ImmunoResearch 109-006-098) was immobilized on a CM5 Sensor Chip via amine coupling chemistry to a level of approximately 5000 RU. A reference cell was treated in the same manner. After amine coupling was complete, all subsequent solutions were flowed over the reference cell and the sample cell in series, except for the capture solution, and the response of the reference cell was subtracted from the sample cell throughout the run.

Each analysis cycle consisted of capture of approximately 250 RU of 12172gL2gH1 1 hIgG4P to the anti Fc surface, injection of analyte for 180 s (at 25° C. at a flow rate of 30 μl per minute), dissociation of analyte for 600 s, followed by surface regeneration (with a 60 s injection of 50 mM HCl, a 30 s injection of 5 mM NaOH, and a further 60 s injection of 50 mM HCl). Human TREM1 ECD analyte (in house, His tagged) was injected at 3-fold serial dilutions in HBS-EP+ running buffer (GE Healthcare) at concentrations of 200 nM to 2.5 nM on the T200, and concentrations of 500 nM to 2 nM on the 8k. Cyno TREM1 ECD analyte (in house, His tagged) was injected at 3-fold serial dilutions in HBS-EP+ running buffer (GE Healthcare) at concentrations of 4100 nM to 17 nM—this was run on the T200 only. Buffer blank injections were included to subtract instrument noise and drift.

Kinetic parameters were determined using a 1:1 binding model using Biacore T200 Evaluation software (version 3.0) or Biacore Insight Evaluation software (version 3.0), as appropriate. 12172gL2gH1 1 hIgG4P was shown to have an affinity of 0.52 nM for human TREM1 and 870 nM for cyno TREM1. The kinetic parameters are summarized in Table 14.

TABLE 14
Kinetic parameters of 12172gL2gH11 hIgG4P binding to human
and cynomolgus TREM1

| Species | kₐ (1/Ms) | k_{d} (1/s) | K_{D} (nM) | n= |
|---|---|---|---|---|
| Human | 1.8E+05 | 9.2E−05 | 0.52 | 2 |
| Cynomolgus | 1.2E+05 | 1.0E−01 | 870 | 1 |

### Example 7. Binding of 12172gL2gH11 hIgG4P to Different Species of TREM1

The kinetics of 12172gL2gH11 hIgG4P binding to various species of TREM1 were measured at 25° C. by surface plasmon resonance on a Biacore T200 instrument. The species tested were human, cynomolgus, rhesus, marmoset, rat, mouse, dog and pig.

A goat anti human IgG, Fc fragment specific antibody (F(ab′)2 fragment, Jackson ImmunoResearch 109-006-098) was immobilized on a CM5 Sensor Chip via amine coupling chemistry to a level of approximately 5000 RU. A reference cell was treated in the same manner. After amine coupling was complete, all subsequent solutions were flowed over the reference cell and the sample cell in series, excepting the capture solution, and the response of the reference cell was subtracted from the sample cell throughout the run.

Each analysis cycle consisted of capture of approximately 250 RU of 12172gL2gH1 1 hIgG4P to the anti Fc surface, injection of analyte for 180 s (at 25° C. at a flow rate of 30 μl per minute), dissociation of analyte for 600 s, followed by surface regeneration (with a 60 s injection of 50 mM HCl, a 30 s injection of 5 mM NaOH, and a further 60 s injection of 50 mM HCl). TREM1 ECD analyte (in house, His tagged) was injected at 3-fold serial dilutions in HBS-EP+ running buffer (GE Healthcare), top concentrations are shown in Table 15, and three-fold serial dilutions were performed to a bottom concentration of 2 nM. Buffer blank injections were included to subtract instrument noise and drift.

Kinetic parameters were determined using a 1:1 binding model using Biacore Insight Evaluation software (version 3.0). 12172 gL2gH11 hIgG4P was shown to have an affinity of 0.54 nM for human TREM1 in this experiment (kinetic parameters summarised in Table 15). A minimal binding response was observed for cynomolgus and rhesus monkey, though insufficient to determine binding kinetics. No binding of 12172gL2gH11 hIgG4P to TREM1 of any other species was detected (as summarised in Table 16).

TABLE 15
Kinetic parameters of 12172gL2gH11 hIgG4P binding to human TREM1

| Species | kₐ (1/Ms) | k_{d} (1/s) | K_{D} (nM) | n= |
|---|---|---|---|---|
| Human | 1.7E+05 | 9.1E−05 | 0.54 | 1 |

TABLE 16
Report point data showing binding of TREM1 species to 12172gL2gH11 hIgG4P. BL denotes binding
late: the average signal 7.5-12.5 s before the end of the TREM1 injection, subtracted from the
average signal 7.5-12.5 s before the start of this injection. SE denotes stability early: the
average signal 7.5-12.5 s after the end of the TREM1 injection is subtracted from the average signal 7.5-12.5
s before the start of this injection. The theoretical Rmax is the signal that would be produced if all captured
antibodies (150 kDa) were fully bound to 2 molecules of TREM1. SR_BL is BL divided by the theoretical
Rmax, SR_SE is SE divided by the theoretical Rmax.

| | Concentration | | | Capture | TREM1 | Theoretical | | |
|---|---|---|---|---|---|---|---|---|
| Species | (nM) | BL (RU) | SE (RU) | level (RU) | MW (kDa) | Rmax (RU) | SR_BL | SR_SE |
| human | 490 | 78 | 78 | 223 | 21 | 62 | 1.3 | 1.3 |
| cynomolgus | 460 | 26 | 16 | 231 | 21 | 65 | 0.4 | 0.3 |
| rhesus | 450 | 12 | 5 | 229 | 21 | 64 | 0.2 | 0.1 |
| marmoset | 510 | 0 | 0 | 224 | 22 | 66 | 0.0 | 0.0 |
| mouse | 520 | 0 | 0 | 228 | 21 | 64 | 0.0 | 0.0 |
| rat | 470 | 0 | 0 | 233 | 21 | 65 | 0.0 | 0.0 |
| pig | 490 | 0 | 0 | 233 | 22 | 68 | 0.0 | 0.0 |
| dog | 460 | 0 | 0 | 226 | 22 | 66 | 0.0 | 0.0 |

### Example 8. Blocking of the TREM1/PGLYRP1 Interaction by 12172gL2gH11 hIgG4P

12172gL2gH1 1 hIgG4P was demonstrated to block the interaction between human TREM1 and human PGLYRP1 at 25° C. by surface plasmon resonance on a Biacore T200 instrument.

A goat anti human IgG, Fc fragment specific antibody F(ab′)₂ fragment, Jackson ImmunoResearch 109-006-098) was immobilized on all four flow cells of an HC30M Sensor Chip (XanTec Bioanalytics) via amine coupling chemistry to a level of approximately 4000 RU. The response of flow cell 1 was subtracted from the response of flow cell 2 throughout the run, similarly the response of flow cell 3 was subtracted from the response of flow cell 4 throughout.

Each analysis cycle consisted of capture of approximately 100 RU of TREM1-Fc (R&D 1278-TR Lot GZF0220071) to the surface of flow cell 2, capture of approximately 150 RU 12172gL2gH1 1 hIgG4P to the surface of flow cell 4, and TREM1 ECD analyte (in house, His tagged) was flowed over the surface of flow cells 3 and 4 for 180 s. A mixture of PGLYRP1 (R&D 2590-PGB, NLC1520031) and PGN (Invivogen tlrl-ksspgn lot KSS-41-01) was flowed over all surfaces for 180 s and the binding monitored, followed by a 300 s dissociation period. The surfaces were then regenerated (with a 60 s injection of 50 mM HCl, a 30 s injection of 5 mM NaOH, and a further 60 s injection of 50 mM HCl).

12172gL2gH1 1 hIgG4P blocked the interaction of PGLYRP1 and TREM1 in the presence and absence of PGN (see Table 17).

TABLE 17
Relative response at the binding late report point. This is calculated during the injection of
PGLYRP1 and PGN: the average signal 7.5-12.5 s before the end of the injection is subtracted
from the average signal 7.5-12.5 s before the start of this injection. This value is then subtracted
from the value in an equivalent cycle with no captured ligand. This shows that TREM1-Fc binds
5 RU of PGLYRP1 alone, and it binds 12 RU of a mixture of PGLYPR1 and PGN, however PGLYRP1 does
not bind TREM1 which has been captured to a surface coated in 12172gL2gH11 hIgG4P.

| All flow cells | | Flow cell 2 | | Flow cell 4 | | |
|---|---|---|---|---|---|---|
| Concentration | Concentration | | Binding | | Concentration | Binding |
| PGLYRP1 (nM) | PGN (μg/ml) | Ligand | late (RU) | Ligand | of TREM1 (nM) | late (RU) |
| 0 | 0 | TREM1-Fc | 0 | 12172 gL2gH11 hIgG4P | 552 | −1.8 |
| 30 | 0 | TREM1-Fc | 4.5 | 12172 gL2gH11 hIgG4P | 552 | −1.8 |
| 0 | 0.55 | TREM1-Fc | −0.5 | 12172 gL2gH11 hIgG4P | 552 | −1.9 |
| 30 | 0.55 | TREM1-Fc | 11.8 | 12172 gL2gH11 hIgG4P | 552 | −1.7 |

### Example 9: Determination of the Binding Interface of Human PGLYRP1 with Human TREM1 by Crystallography

### Crystallography

The human TREM1 IgV domain was complexed with full-length human PGLYRP1 (1:1 molar ratio) in PBS, pH 7.0 at 15 mg/ml and incubated for an hour at 4° C. The proteins were co-crystallized in a hanging drop, vapor diffusion consisting of 0.2ul protein and 0.2 ul reservoir Molecular Dimensions ProPlex screen D7 (15% (w/V) PEG 6000, 100 mM sodium citrate tribasic/sodium hydroxide pH 5.5). 20% ethylene glycol was used for cryo protection.

### X-Ray Diffraction, Data Collection, Structure Determination and Refinement

The cryogenic (100K) X-ray diffraction data were collected remotely at APS 21-ID-F. Raw data frames were indexed, integrated, and scaled using XDS. The Space Group of the crystal was P21, with unit cell parameters, a=58.68 Å, b=98.73 Å, c=60.43 Å, α=90°, β=109.145°, γ=90° to a resolution of 2.55 Å. The quality parameters of the structure were good with overall R-factor of the structure=17.9% and the free R-Factor=22.9%. The protein complex structure was modelled in COOT and refined using PHENIX, including TLS protocol. Water molecules were added and checked by COOT.

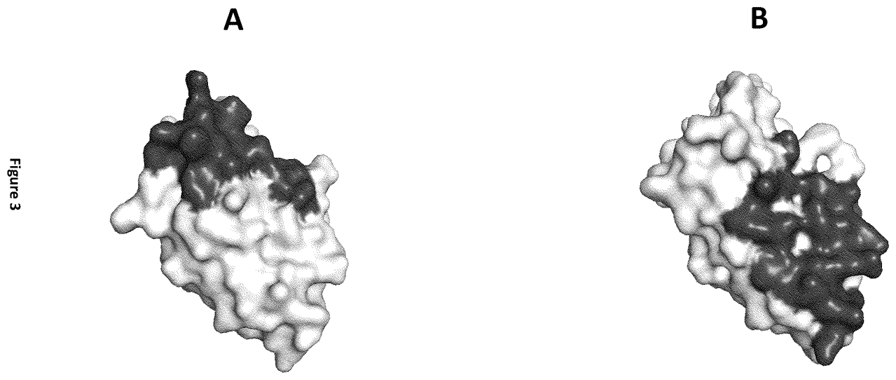

Human TREM1 IgV domain (positions 21-139) (SEQ ID NO: 9) complexed with full-length PGLYRP1 (SEQ ID NO: 10).). The stoichiometry of the crystal complex is 1:2, with a single hTREM1 molecule bound to a PGLYRP1 molecule that is dimerized, despite equimolar mixing. The analysis of intermolecular distances of less than or equal to 4 Å between the IgV-like domain of human TREM1 and hPGLYRP1 was carried out using the program NCONT in the CCP4 program suite. The epitope has been determined crystallographically as follows: residues E27, D42-E46, A49, Y90-L95, and F126 (the positions correspond to SEQ ID NO: 1). FIG. 3A shows structural mapping of the PGLYRP1 ligand binding site on hTREM1: PDBPDB 1SMO.

### Example 10: Determination of the 12172 Rabbit Parental Fab Human TREM1 Epitope by X-Ray Crystallography

### Crystallography

To identify the precise epitope of the 12172 antibody, X-ray crystallography was used. The human TREM1 IgV domain (SEQ ID NO: 9) was complexed with rabbit parental Fab (1:1 molar ratio) in PBS, pH 7.4 and applied to size exclusion. Chaperone Fab 11994 was mixed with the hTREM1: Fab complex at 15 mg/ml and incubated for an hour at 4° C. The proteins were co-crystallized in a hanging drop, vapor diffusion consisting of 0.2 ul protein and 0.1 ul reservoir Molecular Dimensions ProPlex screen A6 (25% (w/V) PEG 1000, 200 mM Sodium chloride, 100m potassium phosphate dibasic/sodium phosphate monobasic pH 6.5). 20% ethylene glycol was used for cryo protection.

### X-Ray Diffraction, Data Collection, Structure Determination and Refinement

The cryogenic (100K) X-ray diffraction data were collected remotely at APS 21-ID-F. Raw data frames were indexed, integrated, and scaled using XDS. The Space Group of the crystal was 1222, with unit cell parameters, a=91.98 Å, b=139.10 Å, c=224.81 Å, α=90°, β=90°, γ=90° to a resolution of 2.60 Å. The quality parameters of the structure were good with overall R-factor of the structure=19.3% and the free R-Factor=22.6% The protein complex structure was modelled in COOT and refined using PHENIX, including TLS protocol. Water molecules were added and checked by COOT.

Human TREM1 IgV domain (positions 21-139) (SEQ ID NO: 9) complexed with rabbit parental Fab (12172) (SEQ ID NO: 19 and 23) was obtained in the presence of a chaperone Fab molecule (11994). Using Hydrogen Deuterium Exchange Mass Spectrometry (HDX-MS), it has been confirmed that the 11994 Fab chaperone used for crystallography of 12172 rabbit parental Fab does not influence 12172 binding to TREM1. The stoichiometry of the crystal complex is 1:1:1, with the Fab epitopes binding to opposite sides on the hTREM1 molecule. The analysis of intermolecular distances of less than or equal to 4 Å between the IgV-like domain of human TREM1 and Fab 12172 was carried out using the program NCONT in the CCP4 program suite. The epitope was determined crystallographically as follows: residues E26-K32, Q35, T36, D38, K40, D42, R97, D127, T134 and G136 (the positions correspond to SEQ ID NO: 1). FIG. 3B shows structural mapping of the Fab 12172 epitope on hTREM1: PDBPDB 1SMO. The epitope was confirmed to be different from the epitope of PGLYRP1 (see Example 9).

### Example 11. Full Length Antibody 12172gL2gH11 hIgG4P—Mammalian Cell Line Development

To demonstrate stable expression of 12172gL2gH1 1 hIgG4P, a stably expressing mammalian cell line was created. A CHO cell line was transfected with the plasmid vector 12172_gL2_ckappa_gH11_IgG4(p). The cell lines were cloned and evaluated for fit to a suitable manufacturing process. To assess the quality and quantity of the protein of interest and to ensure the optimal cell line was selected, the cell line was evaluated in a small-scale model of a manufacturing fed-batch bioreactor. Clonal CHO cell lines were selected that express12172gL2gH1 1 hIgG4P at acceptable levels and containing more than 95% of monomer..

### Example 12. Characterization of Antibody Molecules by Liquid Chromatography-Mass Spectrometry (LC-MS)

The molecular weight (MW) of produced 12172 gL2gH11 (hIgG4P and hIGI LALA) and 12172 gL6gH6 (hIgG4P and hIgG1 LALA) antibody molecules was measured both on the intact molecules (non-reduced) and the separate heavy and light chains (reduced) by LC-MS using a Waters ACQUITY UPLC System with a Xevo G2 Q-ToF mass spectrometer. Samples (˜5g) were reduced with 5 mM tris(2-carboxyethyl) phosphine (TCEP) in 150 mM ammonium acetate at 37° C. for 40 minutes. For non-reduced (intact) measurement, the samples were diluted with PBS pH 7.4 to the same concentration and incubated as above prior to analysis The LC column was a Waters BioResolve™ RP mAb Polyphenyl, 450 Å, 2.7 μm held at 80° C., equilibrated with 95% solvent A (water/0.02% trifluoroacetic acid (TFA)/0.08% formic acid) and 5% Solvent B (95% acetonitrile/5% water/0.02% TFA/0.08% formic acid) at a flow rate of 0.6 mL/minute. Proteins were eluted with a gradient from 5% to 50% solvent B over 8.8 minutes followed by a 95% solvent B wash and re-equilibration. UV data were acquired at 280 nm. MS conditions were as follows: Ion mode: ESI positive ion, resolution mode, mass range: 400-5000m/z and external calibration with NaI.

Data were analyzed using Waters MassLynx™ and MaxEnt Software.

As shown in Tables 18 and 19 the predicted MW from the sequences of 12172 gL2gH11 (hIgG4P and hIgG1 LALA) and 12172 gL6gH6 (hIgG4P and hIgG1 LALA) antibody molecules were consistent with the measured MW for the intact molecules and the heavy and light chains by LC-MS.

TABLE 18
Intact (non-reduced) LC-MS data of 12172 gL2gH11 and 12172 gL6gH6
(hIgG4P and hIgG1 LALA) antibody molecules.

| | | Intact Antibody MW (Da) | | |
|---|---|---|---|---|
| Graft | Format | Expected | Observed | Δm |
| 12172 | hIgG4P | 147482.6 | 147485.0 | 2.4 |
| gL2gH11 | hIgG1 LALA | 147624.9 | 147628.0 | 3.1 |
| 12172 | hIgG4P | 147430.6 | 147433.0 | 2.4 |
| gL6gH6 | hIgG1 LALA | 147572.9 | 147578.5 | 5.6 |

TABLE 19
Reduced LC-MS data of 12172 gL2gH11 and 12172 gL6gH6
(hIgG4P and hIgG1 LALA) antibody molecules.

| | | Light Chain MW (Da) | | | Heavy Chain MW (Da) | | |
|---|---|---|---|---|---|---|---|
| Graft | Format | Expected | Observed | Δm | Expected | Observed | Δm |
| 12172 | hIgG4P | 23651.2 | 23650.8 | −0.4 | 50094.1 | 50095.4 | 1.3 |
| gL2gH11 | hIgG1 | 23651.2 | 23650.6 | −0.6 | 50165.3 | 50166.4 | 1.1 |
| | LALA | | | | | | |
| 12172 | hIgG4P | 23622.2 | 23621.6 | −0.6 | 50097.1 | 50098.2 | 1.1 |
| gL6gH6 | hIgG1 | 23622.21 | 23621.8 | −0.4 | 50168.3 | 50169.4 | 1.1 |
| | LALA | | | | | | |

### Example 13. Thermal Stability (Tm) Measurements

The melting temperature (Tm) or temperature at the midpoint of unfolding was determined using the (i) thermal shift assay or (ii) Differential Scanning Calorimetry (DSC) to assess the conformational stability of the molecules and hence robustness to manufacture and long term stability.

### Thermal Shift Assay

The thermal shift assay was performed on early graft screening and selection, see Example 3.

The fluorescent dye SYPRO® orange was used to monitor the protein unfolding process by binding to hydrophobic regions that become exposed as the temperature increases. The reaction mix contained 5 μL of 30× SYPRO® Orange Protein Gel Stain (Thermofisher scientific, S6651), diluted from 5000× concentrate with test buffer. 45 μL of sample at 0.2 mg/mL, in a common pre-formulation storage buffer, pH 7.4, was added to the dye and mixed. 10 μL of this solution was dispensed in quadruplicate into a 384 PCR optical well plate and was run on a QuantStudio 7 Real-Time PCR System (Thermofisher™). The PCR system heating device was set at 20° C. and increased to 99° C. at a rate of 1.1° C./min. A charge-coupled device monitored fluorescence changes in the wells. Fluorescence intensity increases were plotted, the inflection point of the slope(s) was used to generate apparent midpoint temperatures (Tm). The data is shown in Table 11 and 12 (see Example 3).

### Differential Scanning Calorimetry (DSC)

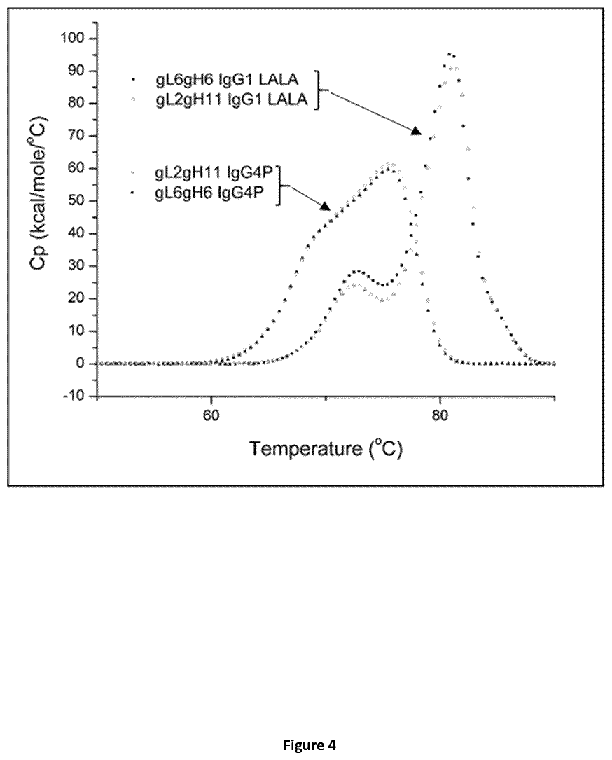

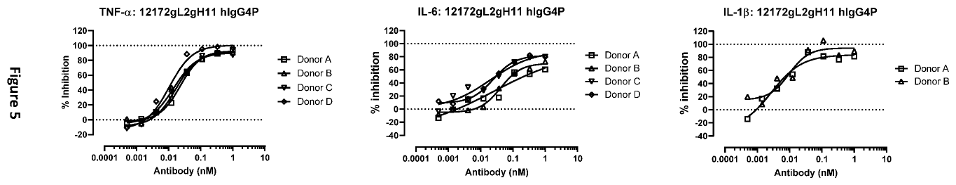

Differential Scanning calorimetry was used to assess thermal stability. All samples were diluted to 10 μM in a common pre-formulation storage buffer, pH7.4 or pH5.0 in a total volume of 400 μL, added to a 96-well plate and centrifuged at 4,000×g for 5 min to remove air bubbles. The plate was run on an automated MicroCal VP DSC (Malvern Panalytical), from 10-100° C., at a rate of 1° C./min, with a pre-scan thermostat of 15 min, filtering period of 8s and in passive feedback mode. Data was buffer subtracted, with manual baseline correction and data fitted to a non-2-state model in Origin 7.0. Transition midpoints (Tm) and onset of unfolding are shown below Table 20 and FIG. 4.

The IgG4P isotypes were fitted to three transitions whilst the IgG1 LALA's were fitted to two transitions, where the Fab and CH3 unfolding were unable to be differentiated. Thermal stability was within the expected ranges for each isotype.

TABLE 20
Summary of Thermal Stability data for 12172 gL2gH11 and 12172
gL6gH6 (hIgG4P and hIgG1 LALA) in a common pre-formulation
storage buffer pH 7.4. Tm1 = CH2 unfolding, Tm2 = Fab unfolding,
Tm3 = CH3 unfolding

| | | Thermal Stability PH 7.4 (° C.) | | | |
|---|---|---|---|---|---|
| Graft | Format | Tm1 | Tm2 | Tm3 | onset |
| gL2gH11 | hIgG4P | 69.94 | 74.30 | 76.84 | 60.93 |
| | hIgG1 LALA | 72.86 | 80.81 | | 65.76 |
| gL6gH6 | hIgG4P | 69.72 | 74.08 | 76.67 | 61.20 |

| | | | |
|---|---|---|---|
| hIgG1 LALA | 73.12 | 80.65 | 66.05 |

### Example 14. Experimental Isoelectric Point (pI) Measurement

The experimental pI was found to be similar for the 12172 gL2gH11 and 12172 gL6gH6 as hIgG4P formats. This was also observed for the hIgG1 LALA molecules. The pI was in a range that was expected to be good for manufacturing steps and formulation buffers. The presence of different charged species was consistent with observations of other therapeutic molecules and attributed to common post-translation modifications, such as C terminal heavy chain removal of lysine.

### Example 15. Hydrophobic Interaction Chromatography (HIC)

Hydrophobic Interaction chromatography (HIC) was used to measure hydrophobicity of 12172 gL2gH11 and 12172 gL6gH6 as hIgG4P formats. HIC separates molecules in order of increasing hydrophobicity. Molecules bind to the hydrophobic stationary phase in the presence of high concentrations of polar salts and desorb into the mobile phase as the concentration of salt decreases. A longer retention time equates to a greater hydrophobicity.

All molecules showed low apparent hydrophobicity (less than 15 minutes retention time). There was no meaningful difference between 12172 gL2gH11 and 12172 gL6gH6 hIgG4P molecules. Similarly, there was no meaningful difference in hydrophobic retention times for the hIgG1 LALA samples. The hIgG4P molecules showed slightly later retention times compared with the corresponding hIgG1 LALA molecules.

### Example 16. Solubility Measurement Using Polyethylene Glycol (PEG) Aggregation Assay

The PEG aggregation assay was used as a mimic of high concentration solubility. PEG is a nonadsorbing, nondenaturing polymer and due to its inert nature, has been used to promote protein precipitation primarily via an excluded volume effect. Samples were exposed to increasing concentrations of PEG 3350; the amount of sample remaining in solution was determined by plotting absorbance at A280 nm. The determination of % PEG concentration at which half the sample had precipitated generated a PEG midpoint (PEGmdpnt) score. This score permitted test molecules to be ranked on apparent native state aggregation propensity, a low PEGmdpnt score (for example ≤10) indicates a greater propensity for native state aggregation.

Stock 40% PEG 3350 (Merck, 202444) solutions (w/v) were prepared in common pre-formulation storage buffers pH 7.4 and 5.0 and a common pre-formulation buffer pH 5.5). A serial titration was performed by an ASSIST PLUS liquid handling robot (INTEGRA 4505), resulting in a range of 40% to 15.4% PEG 3350. To minimize non-equilibrium precipitation, sample preparation consisted of mixing antibody and PEG solutions at a 1:1 volume ratio. 35 μL of the PEG 3350 stock solutions was added to a 96 well v bottom PCR plate (A1 to H1) by a liquid handling robot. 35 μL of a 2 mg/mL sample solution was added to the PEG stock solutions resulting in a 1 mg/mL test concentration and a final PEG 3350 concentration of 20% to 7.7%. This solution was mixed by automated slow repeat pipetting and incubated at 37° C. for 0.5 h to re-dissolve any non-equilibrium aggregates. Samples were then incubated at 20° C. for 24 h. The sample plate was subsequently centrifuged at 4000×g for 1 h at 20° C. 50 μL of supernatant was dispensed into a UV-Star®, half area, 96 well, Clear®, microplate (Greiner, 675801). Protein concentrations were determined by UV spectrophotometry at 280 nm using a FLUOstar® Omega multi-detection microplate reader (BMG LABTECH). The resulting values were plotted using Graphpad prism (version 7.04); the PEG midpoint (PEG_{mdpnt}) score was derived from the midpoint of the sigmoidal dose-response (variable slope) fit.

The data is shown in Table 21 where the higher PEG mid-point (%) equates to greater solubility.

Buffer dependent solubility was observed for the molecules tested. In a common pre-formulation storage buffer pH 7.4, both isotypes (hIgG4P and hIgG1 LALA) of 12172 gL2gH11 and 12172 gL6gH6 exhibited low PEG midpoints scores, indicating low solubility at high concentration. Increased PEG midpoint scores were observed in the common pre-formulation storage buffer pH 5 buffer. Notably all the samples showed substantially improved PEG midpoint scores when formulated in the common pre-formulation buffer pH 5.5. The hIgG1 LALA samples did not precipitate at the highest test concentration of PEG 3350 in this buffer.

TABLE 21
PEG aggregation assay data for 12172 gL2gH11 and 12172 gL6gH6
(hIgG4P and hIgG1 LALA) in the common pre-formulation storage
buffers pH 7.4 and 5.5, and the common pre-formulation buffer pH 5.5.
Higher PEG % midpoint = greater high concentration solubility. NB
*samples showed signs of aggregation at the lowest test concentration
of PEG 3350 (7.7%) therefore accurate PEG midpoints could not
be generated.

| | | % midpoint | | |
|---|---|---|---|---|
| | | common pre- | common | common pre- |
| | | formulation | pre- | formulation |
| | | storage | formulation | storage |
| Graft | Format | buffer pH7.4 | buffer pH 5.5 | buffer pH 5 |
| gL2gH11 | hIgG4P | *<7.7 | 13.0 | 10.0 |
| | hIgG1 | *<7.7 | >20 | 12.2 |
| | LALA | | | |
| gL6gH6 | hIgG4P | *<7.7 | 14.4 | 10.7 |
| | hIgG1 | *<7.7 | >20 | 12.9 |
| | LALA | | | |

### Example 17. Assessment of Protein-Protein Self-Interaction Using AC-SINS (Affinity Capture Self-Interaction Nanoparticle Spectroscopy)

The AC-SINS assay was used to screen the developability of humanized molecules including 12172 gL2gH 11 and 12172 gL6gH6 (as hIgG4P and hIgG1 LALA; also see Example 3) by determining protein-protein self-interaction propensity, hence informing on potential aggregation stability. This was performed in a common pre-formulation storage buffer pH 7.4.

Goat anti human-Fcγ specific capture antibody (Jackson ImmunoResearch) was buffer exchanged into 20 mM sodium acetate, pH4.3, diluted to 0.4 mg/mL and 50 μL added to 450 μL citrate-stabilized 20 nm gold nanoparticles (TedPella, USA) and left overnight at room temperature. The conjugated nanoparticles were blocked with 55 μL PEG methyl ether thiol (average Mn=2,000 (Sigma #729140) for 1 hour, centrifuged at 21,000×g for 6 min, the supernatant removed and resuspended in 20 mM sodium acetate, pH4.3 to a final volume of 150 μL.

The antibody samples were diluted to 22 pg/mL in a common pre-formulation storage buffer, pH7.4 (180 μL) and added to 20 μL of mock supernatant and 200 μL non-specific whole IgG at 222 pg/mL (Jackson ImmunoResearch), vortexed briefly and 72 μL added to a 96-well plate. 8 μL of nanoparticles were added to each well (n=4). Absorbance were read on a BMG plate reader from 500-600 nm, fitted to Lorenzian curves (RShiny) and a common pre-formulation storage buffer-only subtracted from the samples to give Δλmax.

The data is summarized in Table 22 where the higher AX max (nm) value equates to a higher protein-protein self-interaction propensity. The hIgG1 LALA molecules for both 12172 gL2gH11 and 12172 gL6gH6 were found to show less self-interaction than the corresponding hIgG4P molecules as shown by a lower Δλ max (nm). Additionally, 12172 gL6gH6 (hIgG4P and hIgG1 LALA) molecules showed slightly lower Δλ max (nm) values than the 12172 gL2gH11 (hIgG4P and hIgG1 LALA) molecules.

TABLE 22
Self-interaction measurement (AC SINS) for 12172 gL2gH11 and 12172
gL6gH6 (hIgG4P and hIgG1). Low value = less protein-protein
self-interaction.

| Graft | Format | λΔ max (nm) |
|---|---|---|
| gL2gH11 | hIgG4P | 13.15 |
| | hIgG1 LALA | 5.970 |
| gL6gH6 | hIgG4P | 8.58 |
| | hIgG1 LALA | 3.93 |

### Example 18. kD Interaction Parameter Measurement (Colloidal Stability)

The kD interaction parameter was used to assess colloidal stability, where positive and negative values relate to repulsive and attractive intermolecular forces respectively.

Dynamic light scattering (DLS) was performed on a DynaPro III plate reader (Wyatt Technology Corp, Santa Barbara, CA, USA). Samples were diluted in a common pre-formulation storage buffer, pH7.4 or buffer exchanged into a common pre-formulation storage buffer, pH5.0 and diluted from 7 mg/mL to 1 mg/mL in increments of 1 mg/mL. Wells containing buffer were selected as solvent offsets and the measurements performed at 25° C., with the laser power set to 20% and auto-attenuation enabled. Each measurement was the average of five, 5s scans in triplicate (5×3). The Diffusion co-efficient was measured (Dm) and the interaction parameter (kD) calculated according to the equation below, where D₀ represents the diffusion coefficient at infinite dilution.

$$D m = {D}_{0} ( 1 + {K}_{D} C )$$

Equation: D₀ given by Debye plot at Y-intercept. The slope=kD*D₀.

The Diffusion coefficient was measured as a function of protein concentration and the kD used to assess colloidal stability, where positive and negative values suggest repulsive and attractive intermolecular forces respectively. For samples that show attractive forces/self-association, the diffusion coefficient gets larger as a function of protein concentration and this is reflected in a negative kD value. The data is shown in Table 23.

The kD interaction parameter was shown to be less negative (more colloidally stable) for both the hIgG4P and hIgG1 LALA molecules in the common pre-formulation storage buffer pH 5 compared with the data obtained in the common pre-formulation storage buffer pH 7.4. The hIgG1 LALA molecules were shown to be more stable than the corresponding hIgG4P molecules. Additionally, 12172 gL6gH6 (hIgG4P and hIgG1 LALA) molecules exhibited slightly greater colloidal stability than 12172 gL2gH11 (hIgG4P and hIgG1 LALA). This data confirmed the data generated from the AC-SINS assay (see Example 17).

TABLE 23
kD interaction parameter data for 12172 gL2gH11 and 12172 gH6gL6
(hIgG4P and hIgG1 LALA). The more negative value = greater
attraction (higher protein-protein self-interaction).

| | | ml/g | |
|---|---|---|---|
| Graft | Format | PBS | Ac pH 5 |
| gL2gH11 | hIgG4P | −14.7 | −7.6 |
| | hIgG1 LALA | −10.8 | −5.1 |
| gL6gH6 | hIgG4P | −12.4 | −5.5 |
| | hIgG1 LALA | −9.8 | −4.1 |

### Example 19. Effect of Mechanical Stress on Aggregation Stability (Aggregation Assay)

Proteins tend to unfold when exposed to an air-liquid interface, where hydrophobic surfaces are presented to the hydrophobic environment (air) and hydrophilic surfaces to the hydrophilic environment (water). Agitation of protein solutions achieves a large air-liquid interface that can drive aggregation. This assay serves to mimic stresses that the molecule would be subjected to during manufacture (for example ultra-filtration) and to provide stringent conditions in order to try to discriminate between different antibody molecules.

Samples in a common pre-formulation storage buffer pH 7.4 or pH 5 were stressed by vortexing using an Eppendorf Thermomixer Comfort™. Prior to vortexing the concentration was adjusted to 1 mg/mL using the appropriate extinction coefficients (1.42 and 1.43 Abs 280 nm, 1 mg/mL, 1 cm path length for hIgG1 LALA and hIgG4P respectively) and the absorbance at 595 nm obtained using a Varian Cary® 50-Bio spectrophotometer to establish the time zero reading. Each sample was sub-aliquoted into 1.5 mL conical Eppendorf®-style capped tubes (3×250 μL) and subjected to vortexing at 1400 rpm at 25° C. for 24 hours. Aggregation (turbidity) was monitored by measurement of the samples at 595 nm at 3 hours and 24 hours post vortexing using a Varian Cary® 50-Bio spectrophotometer. The data is summarized in Table 24.

Both 12172 gL2gH11 and 12172 gL6gH6 (hIgG4P and hIgG1 LALA) showed good aggregation stability in both buffers (a common pre-formulation storage buffer pH 7.4 and pH 5) at 3 hours post vortexing, that is, no turbidity was observed at 595 nm. At 24 hours it was possible to discriminate between the molecules where 12172 gL2gH11 and 12172 gL6gH6 (hIgG1 LALA) showed greater aggregation stability than the corresponding hIgG4P molecules in both buffers. For the hIgG4P molecules, greater aggregation stability was observed in a common pre-formulation storage buffer at pH 7.4 compared with pH 5. It would be envisaged that 12172 gL2gH11 and 12172 gL6gH6, (as hIgG4P and hIgG1 LALA) would be aggregation stable to shear stress conditions during manufacture, for example ultra-filtration.

TABLE 24
Effect of Stress at an air-liquid interface (turbidity at 595 nm) on 12172
gL2gH11 and 12172 gL6gH6 (hIgG4P and hIgG1) in a common
pre-formulation storage buffer pH 7.4 and pH 5.

| | | OD 595 nm | | | |
|---|---|---|---|---|---|
| | | pH 7.4 | | pH 5 | |
| Graft | Format | 3 h | 24 h | 3 h | 24 h |
| gL2gH11 | hIgG4P | 0.06 | 0.87 | 0.21 | 1.48 |
| | hIgG1 | 0.00 | 0.06 | 0.00 | 0.06 |
| | LALA | | | | |
| gL6gH6 | hIgG4P | 0.01 | 0.67 | 0.20 | 1.68 |
| | hIgG1 | 0.00 | 0.13 | 0.01 | 0.03 |
| | LALA | | | | |

### Example 20. Viscosity Assessment at Different Concentrations for 12172 gL2gH11 (hIgG4P and hIgG1 LALA)

Low viscosity at high antibody concentration is important for sub cutaneous administration of the therapeutic molecule, therefore viscosity at increasing concentrations in a common pre-formulation buffer, pH 5 was obtained to assess suitability for sub cutaneous administration. This was determined for 12172 gL2gH 11 (hIgG4P and hIgG1 LALA).

The study was performed by (i) initial concentration of the samples and (ii) viscosity measurement as detailed below.

Concentration of 12172 gL2gH11 (hIgG4P and hIgG1 LALA).

12 mL of 12172 gL2gH11 hIgG4P (15.2 mg/mL) and 11 mL of 12172 gL2 gH11 IgG1 LALA (15.5 mg/mL) in a common pre-formulation storage buffer pH 5.0 were concentrated using Vivaspin 20 MWCO 30 kDa centrifugal filters (Z14637, Sigma-Aldrich) at 4000×g at 20° C. The samples were centrifuged until a volume of −750 μL was obtained. The retentate solution was recovered and the resulting antibody concentrations were determined using UV absorbance measurements (NanoDrop™1000) at 280 nm. Extinction coefficients of 1.43 mL/(mg cm) for 12172gL2gH11 hIgG4P and 1.42 mL/(mg cm) for 12172 gL2gH11 IgG1 LALA were used.

The antibody samples were then diluted using a common pre-formulation storage buffer pH 5.0 to give a range of concentrations suitable for viscosity testing. The concentration of the diluted antibodies was confirmed by remeasurement of UV absorbance at 280 nm. Concentrations were found to be 158 mg/mL, 94 mg/mL and 52 mg/mL for 12172 gL2gH11 hIgG4P and 144 mg/mL 100 mg/mL, and 45 mg/mL for 12172 gL2gH11 hIgG1 LALA.

Viscosity Measurements of 12172 gL2gH11 (hIgG4P and hIgG1 LALA).

The viscosity at each concentration was measured using Discovery Hybrid Rheometer-1 (DHR-1, TA Instruments) with Peltier plate and liquid cooling system for temperature control, and 20 mm stainless steel parallel plate geometry for measurement. The sample (80 μL) was placed on the center of the Peltier plate, and the viscosity (in mPa·s, or cP) was measured with steady state flow sweep procedure setting at 20° C. with varying shear rates, from 2.87918 to 287.918 s⁻¹. The measured viscosity was averaged when the values at each shear rate points are constant (SD±5%). Both 12172 gL2gH11 hIgG4P and 12172 hIgG1 LALA molecules at different concentration were measured using the instrument, to observe the changes in viscosity regarding the sample concentration. The results are summarized in Table 25.

Both 12172 gL2gH11 hIgG4P and 12172 hIgG1 LALA molecules showed an increasing trend between the concentration and the viscosity coefficient. The viscosity increased from 1.2 to 4.1 cP with the concentration from 52 to 158 mg/ml for 12172 hIgG4P. Similarly, the viscosity for IgG1 LALA molecule increased from 1.4 to 5.4 cP with the concentration from 45 to 144 mg/ml. All these samples showed a constant viscosity coefficient (variability less than 5%) at different shear rates. This results showed that 12172 hIgG4P and 12172 hIgG1 LALA exhibited low viscosity levels at a higher concentrations and therefore could be envisaged to be suitable for subcutaneous administration.

TABLE 25
Average viscosity ((centipoise (cP)) at different concentrations of
12172 gL2gH11 (hIgG4P and hIgG1 LALA) at 20° C. in a
common pre-formulation storage buffer pH 5.

| gL2gH11 IgG4P | | gL2gH11 IgG1 LALA | |
|---|---|---|---|
| Concentration | Viscosity | Concentration | Viscosity |
| (mg/mL) | (cP) | (mg/mL) | (cP) |
| 52 | 1.2 | 45 | 1.4 |
| 94 | 2.1 | 100 | 2.4 |
| 158 | 4.1 | 144 | 5.4 |

### Example 21. Assessment of the Functional Activity of 12172 gL2gH11 hIgG4P Using a Human TREM1 THP1 NF-κB Reporter Cell Line

The purpose of this study was to assess the ability of 12172 gL2gH11 hIgG4P to inhibit PGLYRP1/PGN mediated NF-κB signalling activation through human TREM1. To do this, THP1 monocyte TREM1/DAP12 NF-κB Luciferase reporter cells were used These cells stably express human TREM1, human DAP12 and a NF-κB luciferase reporter gene. PGLYRP1 complexed with soluble peptidoglycan from E. coli (PGN) was used as the TREM1 ligand, which induces NF-κB activation by binding to TREM1. PGN which does not bind to TREM1 also induces NF-κB activation, but to a lesser extent and through an alternative signalling pathway. Inhibition of luciferase activity demonstrates the functional blocking activity of 12172 gL2gH11 hIgG4P in this system.

THP1 monocyte TREM1/DAP12 NF-κB Luciferase reporter cells were cultured in complete media containing selection antibiotics (RPMI+10% FBS+50 μM 2-mercaptoethanol+10 μg/ml blasticidin+1 μg/ml puromycin+200 μg/ml geneticin) using standard tissue culture techniques. Three days before assay set up, the cells were seeded at 10×10⁶ cells in 50 ml complete media (200,000 cells/ml) in a T175 flask, placed flat in the incubator. On the day of the assay, the cells were removed from the flask and transferred to a 50 ml falcon and centrifuged at 300×g for five minutes. Media was removed and the cells were resuspended in 5-10 ml of complete media and counted. Cells were then resuspended at 1×10⁶ cells/ml by adding cell suspension to complete media, and 10 μl/well was added to an assay plate (Corning #3570). 12172 gL2gH11 hIgG4P was serially diluted in complete media in a 384-well dilution plate (Greiner #781281) to a final assay concentration range of 33.3 nM to 1.69 μM. The serial dilution of 12172 gL2gH11 hIgG4P was then transferred to the assay plate (10p1/well) and the assay plate was incubated at 37° C./5% CO₂ for 1 hour. Recombinant human PGLYRP1 (R&D Systems #2590-PGB) was complexed with PGN (Invivogen #tlrl-ksspgn) for one hour at room temperature in sterile DPBS. After one hour, the solution was diluted in complete media, then transferred to the assay plate (10 μl/well) to a final assay concentration 2.5 pg/ml PGLYRP1 and 10 pg/ml PGN. The plate controls (no antibody added) included PGLYRP1/PGN complex and PGN alone, as assay maximum and minimum values, respectively. The assay plate was then incubated at 37° C. 5% CO₂ for 16±2 hours. Following the incubation, luciferase activity was measured using the SteadyGlo Luciferase assay system (Promega #E2520). The Steady-Glo reagent was prepared according to the manufacturer's instructions and 30 μl/well was added to the assay plate. The plate was then centrifuged at 200×g for three minutes and then incubated at room temperature for a further two minutes so that the total incubation time with the SteadyGlo reagent was five minutes. Luminescence was then measured using a Synergy Neo 2 plate reader and the raw luminescence values were used to determine the relative percentage inhibition as compared to the control wells. 4PL curve fitting and the calculation of IC₅₀ values was performed using ActivityBase v9.4.

TABLE 26
Summary of potency, efficacy, and hill slope
values for 12172 gL2gH11 hIgG4P

| Geomean IC₅₀ | Average Emax | Average | |
|---|---|---|---|
| (pM) ± SD | (%) ± SD | Slope ± SD | N* |
| 64 ± 36 | 72 ± 4 | 2.1 ± 0.9 | 3 |

*Values were calculated from three independent experiments.

### Example 22. Efficacy and Potency of 12172 Antibodies in Blocking TREM1-Mediated Pro-Inflammatory Cytokine and Chemokine Release from Primary Human Monocytes

To evaluate the ability of anti-TREM1 12172 variant antibodies to block TREM1 signaling, the release of pro-inflammatory cytokines and chemokines from activated primary human monocytes was measured following 12172 antibody treatment. Monocytes were isolated from cryopreserved peripheral blood mononuclear cells (PBMCs) of healthy human donors by negative selection (Miltenyi, 130-117-337). Monocyte viability and purity was assessed by flow cytometry and exceeded ≥90%. Monocytes were seeded at a density of 5×10⁴ cells per well in 96-well plates (Falcon) and stimulated with pre-complexed peptidoglycan from Bacillus subtilis (PGN-BS; 3 pig/ml; Invivogen, tlrl-pgnb3) and recombinant human peptidoglycan recognition protein 1 (PGLYRP1; 1 pig/ml; R&D Systems, 2590-PGB) to activate TREM1. Cell supernatants were collected after 24 hours for measurement of pro-inflammatory cytokine release (TNF-α, IL-6, IL 1P) by homogeneous time resolved fluorescence© technology (HTRF*; Cisbio).

As shown in Table 27, the 12172 gL2gH11 hIgG4P variant was the most potent in inhibiting TREM1-mediated release of TNF-α (Geomean IC₅₀=15 μM), IL-6 (Geomean IC₅₀=27 μM) and IL-1β (Geomean IC₅₀=5 μM) from primary human monocytes. As shown in FIG. 5, the potency of 12172 gL2gH11 hIgG4P in primary human monocytes was observed across donors.

To further evaluate the ability of anti-TREM1 12172 variant antibodies in blocking TREM1-mediated pro-inflammatory cytokine and chemokine release, supernatants from primary human monocytes treated with anti-TREM1 antibodies (1 nM) and activated with pre-complexed PGN-BS/PGLYRP1 were quantitatively analyzed using two multiplex immunoassays: the MILLIPLEX© Human Cytokine/Chemokine/Growth Factor Panel A (Merck Millipore, HCYTA-60K-PX48) and a custom LegendPlex panel (Biolegend).

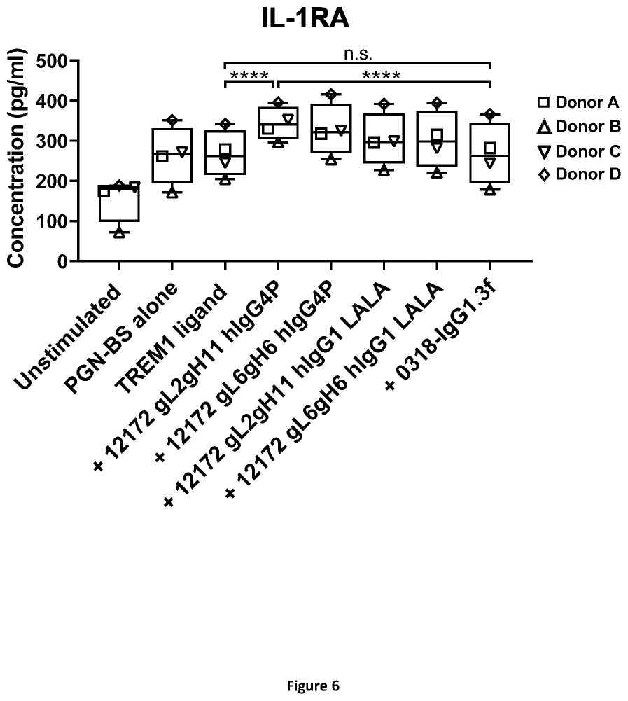

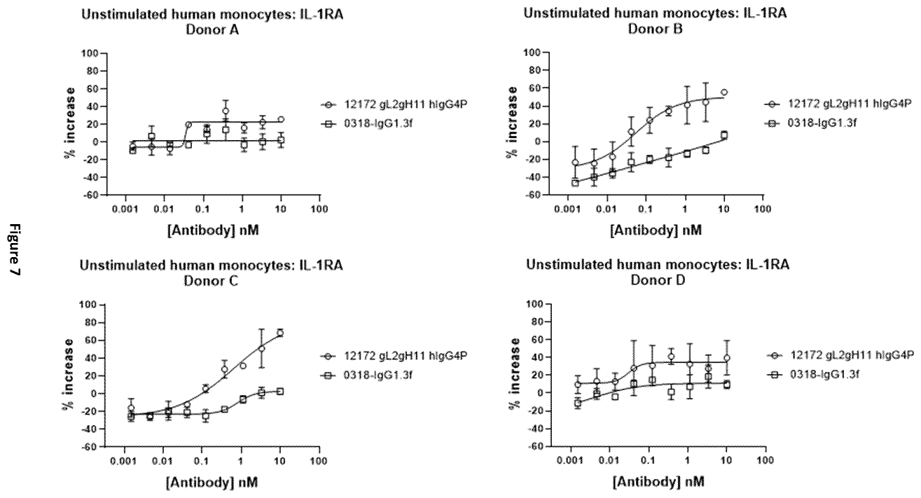

As shown in Table 28, inhibition of TREM1 with different 12172 antibody variants strongly decreased the release of multiple cytokines and chemokines (CCL-3, CCL-20, CXCL-9, G-CSF, GM-CSF, IFN-γ, IL-1α, IL-1μ, IL-6, IL-10, IL-12p40, IL-15, IL-18, IL-27, TNF-α, TNF-β) from activated primary human monocytes (n=4 donors). The 12172 gL2gH11 hIgG4P variant was the most efficacious 12172 variant with percent inhibition values ranging between 57-110%. As shown in FIG. 6, 12172 gL2gH11 hIgG4P significantly increasedthe release of7IL-Rantagonist (IL-1RA), anegative regulator of the IL-i pathway, from primary monocytes across donors. IL-1RA is monogenically (mutations causing low levels of IL-1RA) linked to severe systemic autoimmune disease. Single nucleotide polymorphisms in IL-1RN (encoding for IL-1RA) have been identified in ALS patients. Higher circulating IL 1RA levels are significantly associated with lower risk of ALS (Yuan et al. 2020 Eur J Neurol). IL 1RA levels are also significantly decreased in the cerebrospinal fluid of AD patients compared to healthy controls (Tarkowski et al. 2001 Dement Geriatr Cogn Disord). In contrast to 12172 gL2gH11 hIgG4P, a prior art ani-TREM2 antibody (0318-IgG8.3f) had no effect on IL-1RA release from primary monocytes.

TABLE 27
Potency of different 12172 variants on TNF-α,
IL-6 and IL-1β release

| | TNF-α | IL-6 | IL-1β |
|---|---|---|---|
| | (n = 4) | (n = 4) | (n = 2) |
| | Geo IC50 | Geo IC50 | Geo IC50 |
| Description | (pM) | (pM) | (pM) |
| 12172 gL2gH11 hIgG4P | 15 | 27 | 5 |
| 12172 gL6gH6 hIgG4P | 26 | 36 | 28 |
| 12172 gL2gH11 hIgG1 LALA | 43 | 112 | 32 |
| 12172 gL6gH6 hIgG1 LALA | 54 | 178 | 25 |

TABLE 28
Efficacy (percentage inhibition) of different 12172 antibody
variants on the release of cytokines and chemokines

| | 12172 | 12172 | 12172 | 12172 |
|---|---|---|---|---|
| | gL2gH11 | gL6gH6 | gL2gH11 | gL6gH6 |
| | hIgG4P | hIgG4P | hIgG1 LALA | hIgG1 LALA |
| CCL-3 | 57 | 38 | 48 | 26 |
| CCL-20 | 69 | 56 | 65 | 66 |
| CXCL-9 | 77 | 73 | 72 | 68 |
| G-CSF | 92 | 88 | 89 | 86 |
| GM-CSF | 91 | 89 | 91 | 89 |
| IFN-γ | 59 | 57 | 60 | 53 |
| IL-1α | 110 | 107 | 112 | 110 |
| IL-1β | 98 | 96 | 96 | 97 |
| IL-6 | 64 | 57 | 58 | 55 |
| IL-10 | 86 | 83 | 77 | 73 |
| IL-12p40 | 96 | 96 | 96 | 95 |
| IL-15 | 78 | 69 | 86 | 92 |
| IL-18 | 87 | 78 | 85 | 81 |
| IL-27 | 81 | 57 | 88 | 74 |
| TNF-α | 94 | 93 | 94 | 94 |
| TNF-β | 65 | 63 | 62 | 60 |

### Example 23: Efficacy and potency of 12172 gL2gH11hIgG4P in increasing IL-1RA release from unstimulated primary human monocytes

Having observed that 12172 gL2gH11 hIgG4P significantly increased the release of IL-1RA from TREM1 ligand-stimulated human monocytes, its effects on IL-1RA release from unstimulated human monocytes was also assessed. Human monocytes were isolated and seeded as described previously in Example 22 and antibodies added for 24 hours prior to collection of supernatants for IL-1RA measurement using the IL-1RA Quantikine ELISA kit (R&D Systems).

As shown in Table 29 and FIG. 7, 12172 gL2gH1 1 hIgG4P dose-dependently increased the release of IL-1RA in unstimulated primary human monocytes. In contrast to 12172 gL2gH11 hIgG4P, another prior art anti-TREM1 antibody (0318-IgG1.3f) had no effect on IL-1RA release from unstimulated primary human monocytes.

TABLE 29
Efficacy and potency of 12172 gL2gH11 hIgG4P and 0318-IgG1.3f
in increasing IL-1RA release from unstimulated
primary human monocytes.

| 12172 gL2gH11 | | | |
|---|---|---|---|
| hIgG4P | | 0318-IgG1.3f | |
| % | IC50 | % | IC50 |
| increase | (pM) | increase | (pM) |

| Donor A | n.d. | | n.d. |
|---|---|---|---|
| Donor B | 50 | 54 | n.d. |
| Donor C | 83 | 624 | n.d. |
| Donor D | 35 | 30 | n.d. |

* n.d. = non-detectable

### Example 24. Efficacy of 12172 gL2gH11 hIgG4P in Blocking TREM1-Mediated Pro-Inflammatory Cytokine and Chemokine Release from PBMCs of Alzheimer's Disease (AD) and Amyotrophic Lateral Sclerosis (ALS) Patients

Neurodegeneration and neural inflammation in AD and ALS is associated with elevated levels of multiple pro-inflammatory cytokines and chemokines in the CSF and blood of patients. For example, levels of TNF-α, IL-6 and IL-1β are significantly increased in the blood of ALS patients (Hu et al. 2017 Sci Rep) while CCL-3, G-CSF and TNF-α are elevated in the CSF of ALS patients (Chen et al. 2018 Front Immunol), all factors we observed to be decreased by TREM1 inhibition in human monocytes.

To evaluate the efficacy of anti-TREM1 12172 gL2gH 11 hIgG4P to block TREM1 signaling in patient-derived cells, the release of pro-inflammatory cytokines and chemokines was measured in PBMCs from AD and ALS patients following TREM1 activation. PBMCs were isolated by density gradient centrifugation from whole blood of AD and ALS patients and corresponding matched healthy controls. PBMCs were seeded at a density of 1×10⁵ cells per well in 96-well plates (Falcon), pre-treated for 1 hour with 12172 gL2gH11 hIgG4P (1 nM) and stimulated with pre-complexed peptidoglycan from Bacillus subtilis (PGN-BS; 3 μg/ml; Invivogen, tlrl-pgnb3) and recombinant human peptidoglycan recognition protein 1 (PGLYRP1; 1 μg/ml; R&D Systems, 2590-PGB) to activate TREM1. Cell supernatants were collected after 24 hours for measurement of pro-inflammatory cytokine and chemokine release using homogeneous time resolved fluorescence® technology (HTRF®; Cisbio) and the MILLIPLEX® Human Cytokine/Chemokine/Growth Factor Panel A (Merck Millipore, HCYTA-60K-PX48).

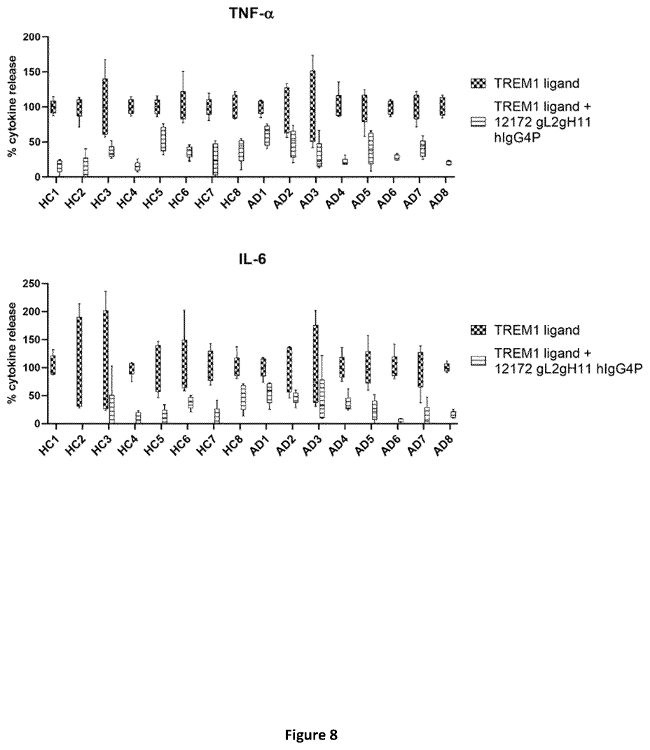

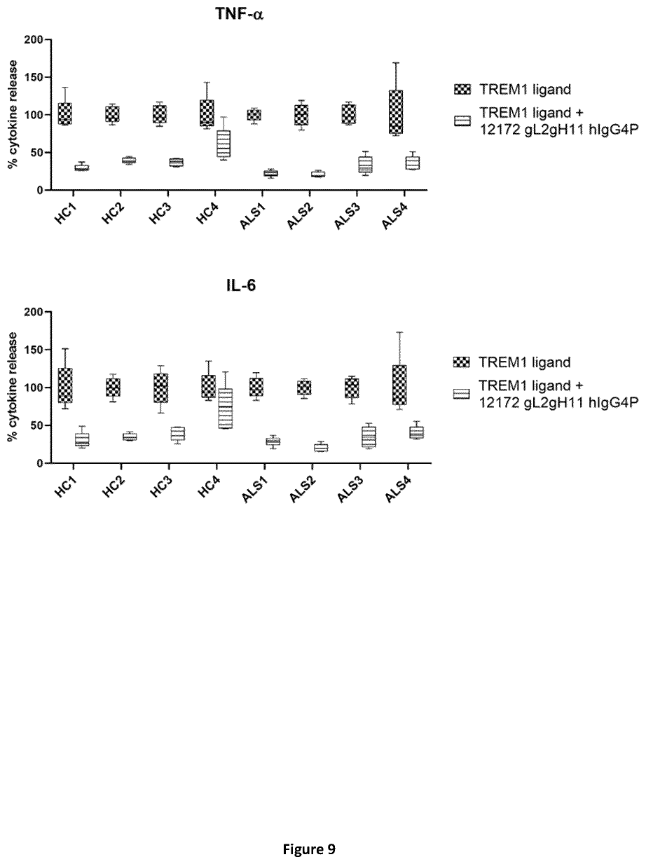

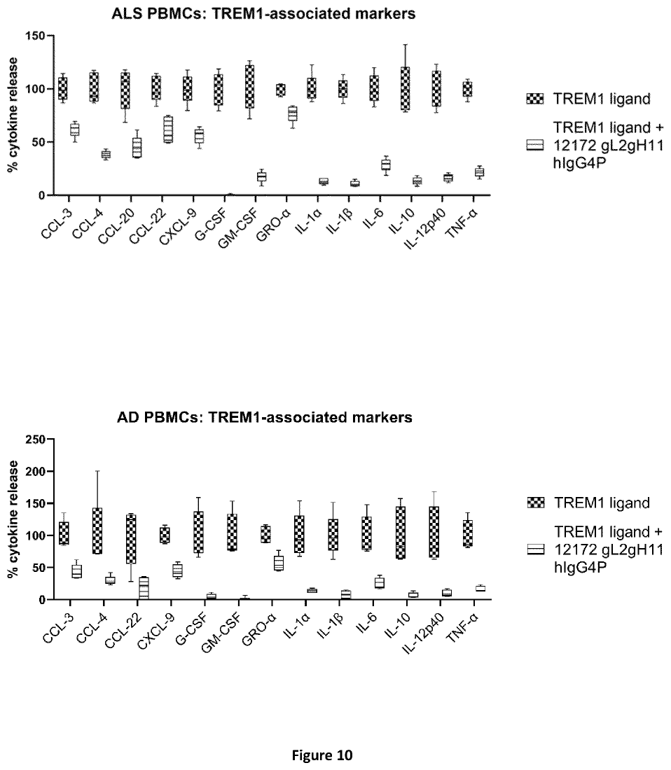

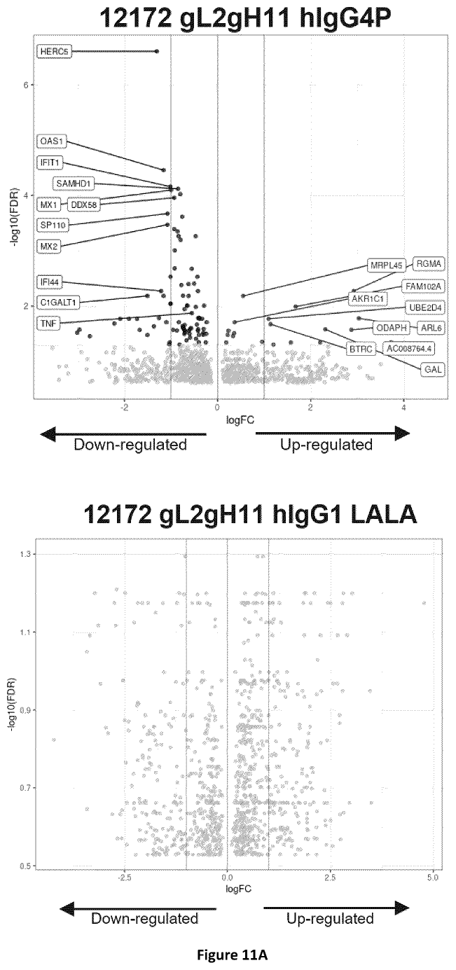

As shown in FIG. 8 and Table 30, 12172 gL2gH11 hIgG4P was efficacious in inhibiting TREM1-mediated TNF-α release (inhibition of 66%±15) and IL-6 release (70%±17) from PBMCs of AD patients. As shown in FIG. 9 and Table 31 12172 gL2gH11 hIgG4P was also efficacious in inhibiting TREM1-mediated TNF-α release (inhibition of 72%±7) and IL-6 release (69%±9) from PBMCs of ALS patients. As shown in FIG. 10, inhibition of TREM1 with 12172 gL2gH11 hIgG4P also strongly decreased the release of multiple cytokines and chemokines (CCL-3, CCL-4, CCL-20, CCL-22, CXCL-9, G-CSF, GM-CSF, GRO-a, IL-1α, IL-1μ, IL-6, IL-10, IL-12p40, TNF-α) from ALS PBMCs (representative of n=4 donors) and AD PBMCs (representative of n=5 donors).

TABLE 30
Efficacy of 12172 gL2gH11 hIgG4P on TNF-α and IL-6
release from healthy control and AD PBMCs.

| | TNF-α | | IL-6 | |
|---|---|---|---|---|
| | % inhibition | S.D. | % inhibition | S.D. |
| Healthy control (HC) n = 8 | 79 | 16 | 85 | 18 |
| Alzheimer's disease (AD) | 66 | 15 | 70 | 17 |
| n = 8 | | | | |

TABLE 31
Efficacy of 12172 gL2gH11 hIgG4P on TNF-α and IL-6 release
from healthy control and ALS PBMCs.

| | TNF-α | | IL-6 | |
|---|---|---|---|---|
| | % inhibition | S.D. | % inhibition | S.D. |
| Healthy control (HC) n = 4 | 59 | 13 | 56 | 19 |
| Amyotrophic lateral sclerosis | 72 | 7 | 69 | 9 |
| (ALS) n = 4 | | | | |

### Example 25. Transcriptomic Profiles of Human Monocytes Following Stimulation with TREM1 Ligand Complex or Apoptotic iPSC-Derived Human Motor Neurons and Treatment with 12172 Antibody Variants

To further characterize the cellular profiles of anti-TREM1 12172 antibody variants, transcriptomic analysis was performed on human monocytes stimulated with TREM1 ligand complex or apoptotic induced pluripotent stem cell (iPSC)-derived human motor neurons, an ALS disease-relevant ligand. Monocytes were isolated from cryopreserved peripheral blood mononuclear cells (PBMCs) of healthy human donors (n=8) by negative selection (Miltenyi, 130-117-337). Monocyte viability and purity was assessed by flow cytometry and exceeded ≥90%. Monocytes were seeded at a density of 2×10⁶ cells per well in 6-well plates (Falcon) and pre-treated for 1 hour with 12172 antibody variants (1 nM). Monocytes were then stimulated for 4 hours with (i) pre-complexed peptidoglycan from Bacillus subtilis (PGN-BS; 3 μg/ml; Invivogen, tlrl-pgnb3) and recombinant human peptidoglycan recognition protein 1 (PGLYRP1; 1 μg/ml; R&D Systems, 2590-PGB) to activate TREM1 or (ii) ultraviolet light-induced apoptotic iPSC-derived human motor neurons. RNA was isolated using the RNeasy Plus Mini Kit (Qiagen) and RNA quality assessed using Experion™ RNA analysis kits (Bio-Rad). Sequencing libraries were prepared using NEBNext Ultra II Directional RNA Library Prep Kit (New England BioLabs) and samples sequenced using Illumina NovaSeq6000.

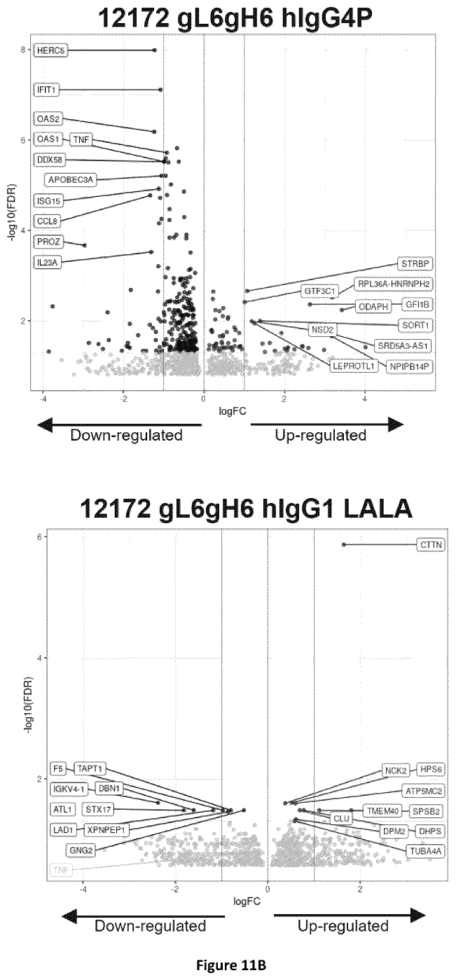

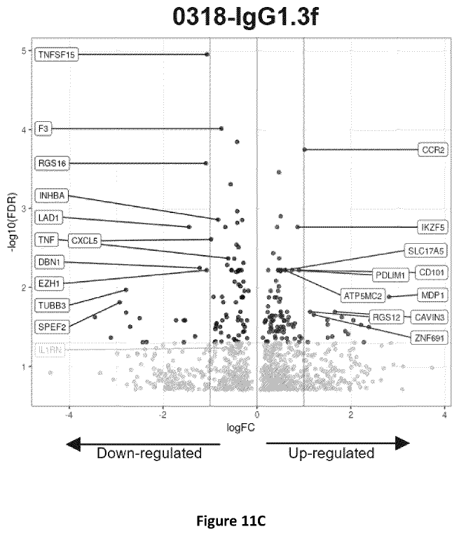

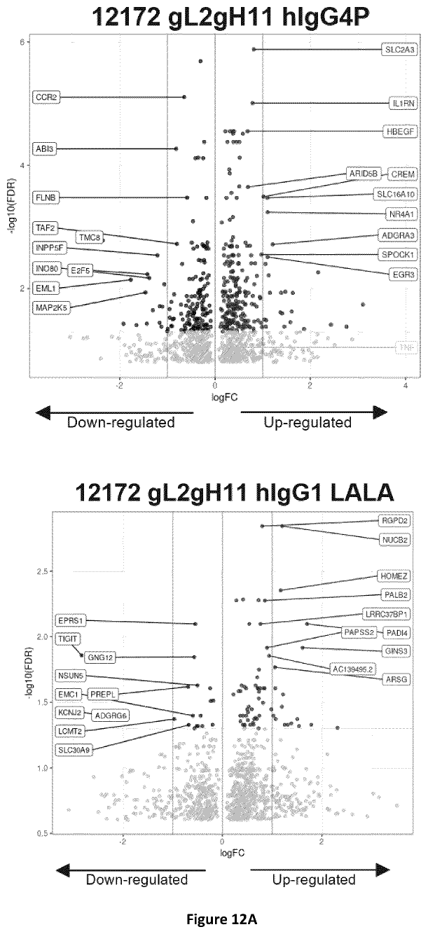

As shown in FIG. 11 (A and B) and Table 32, following TREM1 ligand complex stimulation, the highest number of significant differentially expressed genes (DEGs) was observed with hIgG4P formats of 12172 gL2gH11 and 12172 gL6gH6. Among the top DEGs, the two 12172 hIgG4P antibodies also showed a similar transcriptome profile (e.g. down-regulation of HERC5, OAS], DDX58, TNF-α). These profiles were in contrast to a prior art anti-TREM1 antibody (0318-IgG1.3f) (FIG. 11C). In addition, as shown in Table 33, there were 111 genes significantly up-regulated and 121 genes significantly down-regulated in 12172 gL2gH11 hIgG4P-treated monocytes when compared to 0318-IgG1.3f-treated monocytes.

TABLE 32
Number of differentially expressed genes (DEGs), considering a false
discovery rate (FDR) of 0.05, following treatment of human
monocytes with 12172 antibody variants and stimulation with TREM1
ligand complex (compared to ligand or isotype control).

| | | Up-regulated genes | | Down-regulated genes | |
|---|---|---|---|---|---|
| | | Ab/ligand | Ab/isotype | Ab/ligand | Ab/isotype |
| 12172 gL2gH11 | hIgG4P | 6 | 19 | 4 | 86 |
| | hIgG1 | 0 | 0 | 0 | 0 |
| | LALA | | | | |
| 12172 gL6gH6 | hIgG4P | 20 | 71 | 63 | 255 |
| | hIgG1 | 9 | 10 | 47 | 9 |
| | LALA | | | | |

| | | | | |
|---|---|---|---|---|
| 0318-IgG1.3f | 9 | 99 | 38 | 82 |

TABLE 33
Number of DEGs, considering a FDR of 0.05, between 12172
gL2gH11 hIgG4P and 0318-IgG1.3f following stimulation
of human monocytes with TREM1 ligand complex.
12172 gL2gH11 hIgG4P:0318-IgG1.3f

| Up-regulated genes | Down-regulated genes |
|---|---|
| 111 | 121 |

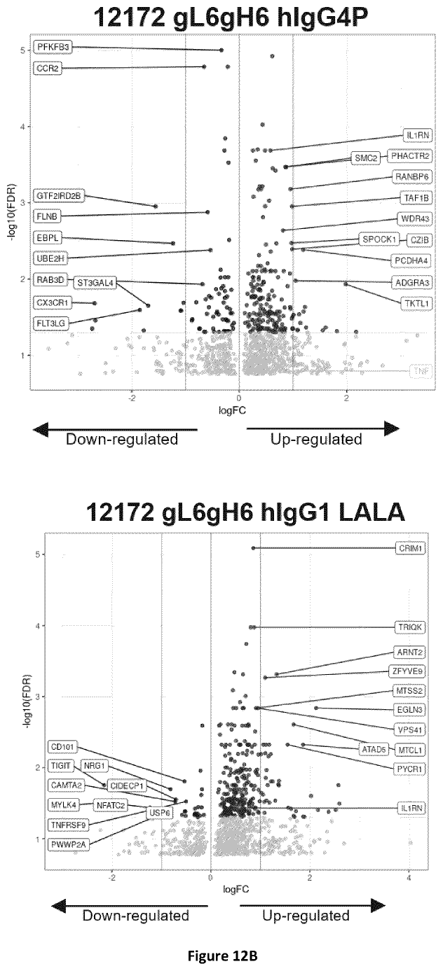

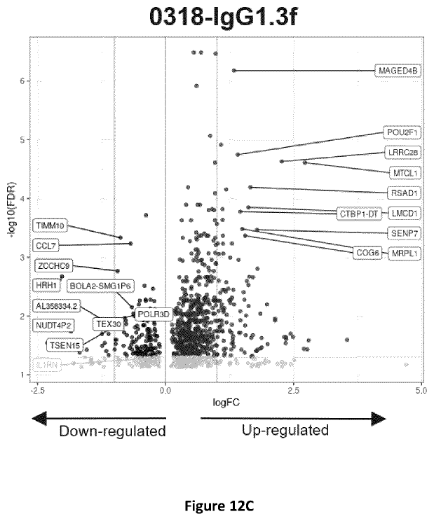

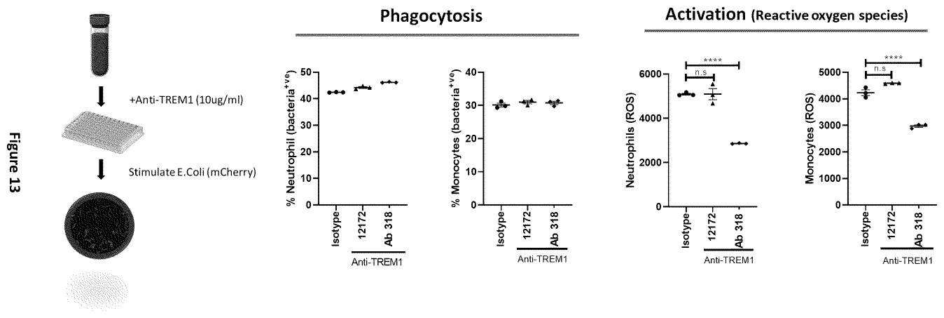

As shown in FIG. 12 (A and B) and Table 34, there were a higher number of DEGs following stimulation with apoptotic iPSC-derived human motor neurons in comparison to TREM1 ligand complex stimulation. Among the top DEGs, the two 12172 hIgG4P antibodies showed some overlap including for example down-regulation of CCR2 and up-regulation of IL-1RN, the gene encoding IL-1RA. In contrast, the top DEGs were different with a prior art anti-TREM1 antibody (0318-IgG1.3f) (FIG. 12C) and no significant up-regulation of IL-1RN was observed with this antibody. In addition, as shown in Table 35, there were 598 genes significantly up-regulated and 808 genes significantly down-regulated in 12172 gL2gH11 hIgG4P-treated monocytes when compared to 0318-IgG1.3f-treated monocytes.

TABLE 34
Number of DEGs, considering an FDR of 0.05, following treatment
of human monocytes with 12172 antibody variants and stimulation
with apoptotic iPSC-derived human motor neurons
(compared to ligand or isotype control).

| | | Up-regulated | | Down-regulated | |
|---|---|---|---|---|---|
| | | genes | | genes | |
| | | Ab/ligand | Ab/isotype | Ab/ligand | Ab/isotype |
| 12172 | hIgG4P | 420 | 221 | 453 | 151 |
| gL2gH11 | hIgG1 LALA | 563 | 64 | 728 | 19 |
| 12172 | hIgG4P | 484 | 183 | 253 | 69 |
| gL6gH6 | hIgG1 LALA | 836 | 280 | 725 | 29 |

| | | | | |
|---|---|---|---|---|
| 0318-IgG1.3f | 674 | 623 | 545 | 139 |

TABLE 35
Number of DEGs, considering a FDR of 0.05, between 12172 gL2gH11
hIgG4P and 0318-IgG1.3f following stimulation of human monocytes
with apoptotic iPSC-derived human motor neurons.
12172 gL2gH11 hIgG4P:0318-IgG1.3f

| Up-regulated genes | Down-regulated genes |
|---|---|
| 598 | 808 |

### Example 26. Efficacy of 12172 Antibody in Blocking TREM1-Mediated Phagocytosis and Production of Reactive Oxygen Species (ROS) by Primary Human Monocytes and Neutrophils

To assess the impact of blocking TREM1 signaling on anti-microbial immune responses, both phagocytosis and ROS production from activated primary human monocytes and neutrophils in whole blood was evaluated by flow cytometry. To examine ROS production, dihydrorhodamine-123 (5 μg/ml) was added to blood (25 μl) from healthy human donors for 5 minutes prior to being preincubated with 12172 gL2gH11 hIgG4P or 0318-IgG1.3f antibodies (10 μg/ml) for an additional 30 minutes. Whole blood samples were then cultured with 1×10⁶ mCherry expressing bacteria for 1 h. Samples were washed, stained with surface antibodies for CD45 and CD14 to discriminate neutrophils and monocytes by flow cytometry.

As shown in FIG. 13, the 12172 gL2gH 11 hIgG4P variant did not impair bacterial clearance by neutrophils or monocytes, nor impact their ability to produce reactive oxygen species. In contrast, analysis of the 0318-IgG1.3f revealed a significant reduction in E. coli induced ROS production by both neutrophils and monocytes. The phagocytic capacity of both immune cell types was not significantly influenced by 0318-IgG1.3f.

### Example 27: Efficacy and Potency of 12172 Antibodies in Blocking TREM1-Mediated Activation Of Spleen Tyrosine Kinase (SYK)

Association of TREM1 with its adaptor protein DAP-12 leads to phosphorylation of DAP-12 and subsequent recruitment and phosphorylation of spleen tyrosine kinase (SYK; Carrasco et al. 2018 Cell Mol Immunol). SYK has previously been implicated in driving TREM1-mediated neuroinflammatory injury (Xu et al. 2019 Cell Death Dis) and is known to be activated following amyloid-P deposition and formation of pathological tau species (Schweig et al. 2017 Acta Neuropathol Commun). To evaluate the ability of anti-TREM1 12172 variant antibodies to block TREM1-mediated SYK activation, phosphorylated SYK (pSYK) levels were measured in Flp-In™ 293 cells stably expressing human TREM1 and human DAP-12. Cells were seeded at a density of 25,000 cells per well in 384-well plates (Greiner), pre-treated for 1 hour with 12172 variant antibodies or isotype antibodies and stimulated with pre-complexed peptidoglycan from Escherichia coli (PGN-EC; 5 μg/ml; Invivogen, tlrl-pgnb3) and recombinant human peptidoglycan recognition protein 1 (PGLYRP1; 2.5 μg/ml; R&D Systems, 2590-PGB) to activate TREM1. Protein lysates were collected after 30 mins for measurement of pSYK levels using the AlphaLISA SureFire Ultra p-SYK (Tyr525/526) Assay Kit® (PerkinElmer).

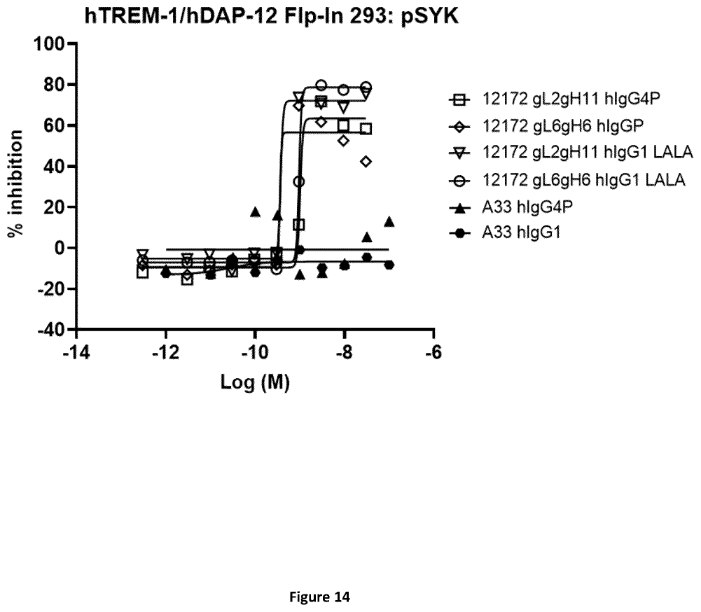

As shown in Table 36 and Figure. 14, all four 12172 variant antibodies were efficient (Emax =57-72%) and potent (357-1015 μM) in blocking SYK activation following TREM1 activation whereas A33 isotype antibodies showed no activity.

TABLE 36
Efficacy and potency of 12172 variant anti-TREM1 antibodies
in blocking SYK activation in hTREM1/hDAP-12
Flp-In 293 cells (ND—non-detectable)

| | hTREM1/hDAP-12 Flp-In 293: | |
|---|---|---|
| | pSYK inhibition | |
| Description | Emax (%) | IC50 (pM) |
| 12172 gL2gH11 hIgG4P | 63 | 1015 |
| 12172 gL6gH6 hIgG4P | 57 | 357 |
| 12172 gL2gH11 hIgG1 LALA | 72 | 372 |
| 12172 gL6gH6 hIgG1 LALA | 79 | 960 |
| A33 hIgG1 | ND | ND |
| A33 hIgG4P | ND | ND |

### Example 28: 12172 gL2gH11 hIgG4P Cell Surface TREM1 Affinity Measurement

The kinetics of 12172 gL2gH 11 hIgG4P binding to human or cynomolgus TREM1 expressed on live cells was measured at 25° C. using LigandTracer. Two HEK293 polyclonal cell lines were developed in-house to express either human or cynomolgus TREM1, and the parental normal adherent HEK293 were used as control cells. All three cell types were maintained in growth medium DMEM (Gibco, 21969-035) supplemented with Foetal Calf Serum (Invitrogen, 10082), GlutaMAX (Gibco, 35050061), and to maintain selection in the TREM1 polyclonal cell lines, 0.5 mg/ml Geneticin (Gibco, 10131-027) was additionally included. The day before an experiment 1.4×10⁶ cells were seeded into each quarter of a LigandTracer MultiDish 2×2 (Ridgeview, 1-04-204-5) previously coated according to manufacturer's instructions with poly-D-lysine (Gibco, A38904-01), and incubated overnight at 37° C./5% CO₂. TREM1 expressing cells were seeded in one quarter of each dish compartment, and negative expressing control cells in the other. The next morning, the medium was exchanged for exactly 1.8 ml fresh growth medium (without geneticin) in each dish compartment (half), and placed in the LigandTracer instrument. Rotation was started to record baseline readings for approximately 20 minutes or until stable. Rotation was halted and AlexaFluor647-labelled 12172 gL2gH11 hIgG4P (in house) was added at a concentration of 0.5 nM, a concentration close to the expected 12172 gL2gH11 hIgG4P K_{D}. Rotation was restarted, and fluorescent measurements representing the real-time binding of the antibody to the cells were recorded until curvature indicating a degree of equilibrium was observed (taking approximately 2 hrs). Two further additions of antibody were made in this manner at 1.5 nM and 5 nM, each ˜3 times higher than the last. Finally, all medium containing the antibody was removed, and replaced with fresh medium. Rotation and measurements were continued until the dissociation signal had dropped by at least 10% or continued overnight if the dissociation was slow. Affinity measurements were analyzed and calculated within the LigandTracer “TraceDrawer” software (version 1.9.2). Raw data readings for binding of 12172 gL2gH 11 hIgG4P to TREM1-expressing cells were first normalized by subtracting the equivalent reading from binding to the control cells. The subtracted traces were evaluated using the software's 1:1 binding model. Alternative models were considered if the 1:1 model was not appropriate for the data traces. 12172 gL2gH 11 hIgG4P was shown to have an affinity of 16.5 μM for human TREM1 and a weaker affinity, around 300 times weaker, for cyno TREM1. The kinetic parameters are summarized in Table 37 and 38. 12172 gL2gH 11 hIgG4P showed binding that was well represented by the 1:1 model. Slow dissociation rates are difficult for the LigandTracer instrument to measure, being towards the limit of the accurate range, but the five replicate experiments gave similar data. The binding of 12172 gL2gH11 hIgG4P to cynomolgus TREM1 was noticeably more complex and did not fit a 1:1 binding model. A 1:2 model, or 1:1-Two State model better represented the data and gave similar affinity values (not all data shown), although further experiments would be required to determine which of these alternative fits correctly describes the binding. However, in general the affinity of 12172 gL2gH 11 hIgG4P for cynomolgus TREM1 compared to human TREM1 was clearly reduced, by approximately 300 times. In conclusion, 12172gL2gH1 1 hIgG4P displayed stronger affinity to cell surface human TREM1 compared to the soluble human TREM1 ECD (Example 6) due to binding avidity on cells, with both methods (Biacore and LigandTracer) showing considerably weaker affinity of 12172gL2gH1 1 hIgG4P to cynomolgus TREM1 compared to human TREM1.

TABLE 37
Kinetic parameters of 12172 gL2gH11 hIgG4P binding to human
TREM1. U-values represent the quality of the fit model to the
data. A low U-value of less than 10% is considered a
good fit, values >20% are considered poor.

| 1:1 fit model | ka (1/(M*s)) | kd (1/s) | K_{D} (pM) | U-value: kd (%) |
|---|---|---|---|---|
| N = 1 | 3.50E+05 | 6.80E−06 | 19.4 | 3.6 |
| N = 2 | 3.56E+05 | 8.89E−06 | 25.0 | 4.3 |
| N = 3 | 2.44E+05 | 3.71E−06 | 15.2 | 10.4 |
| N = 4 | 2.38E+05 | 2.97E−06 | 12.5 | 3.8 |
| N = 5 | 4.43E+05 | 5.88E−06 | 13.3 | 5.7 |
| Average | 3.26E+05 | 5.65E−06 | 17.1 | |
| SD | 8.61E+04 | 2.39E−06 | 5.2 | |
| Geometric mean | 3.17E+05 | 5.23E−06 | 16.5 | |

TABLE 38
Kinetic parameters of 12172 gL2gH11 hIgG4P binding to cyno TREM1. U-values represent
the quality of the fit model to the data. A low U-value of less than 10% is considered
a good fit, values >20% are considered poor. The 1:2 model generates two sets
of affinity values, describing the two contributing binding events.

| | ka 1 | | K_{D} 1 | ka 2 | | K_{D} 2 | U-value: |
|---|---|---|---|---|---|---|---|
| 1:2 fit model | (1/(M*s)) | kd 1 (1/s) | (pM) | (1/(M*s)) | kd 2 (1/s) | (pM) | kd (%) |
| N = 1 | 8.87E+05 | 7.58E−03 | 8550 | 1.08E+05 | 8.69E−06 | 80.8 | 17.7 |
| N = 2 | 3.12E+05 | 1.84E−03 | 5910 | 7.27E+04 | 3.08E−05 | 423 | 4 |
| N = 3 | 4.69E+05 | 1.92E−03 | 4090 | 5.97E+04 | 3.13E−05 | 524 | 3.6 |
| N = 4 | 3.00E+05 | 1.78E−03 | 5940 | 5.97E+04 | 2.36E−06 | 39.5 | 11.8 |
| N = 5 | 7.06E+05 | 1.90E−03 | 2690 | 3.27E+04 | 4.03E−06 | 124 | 6.5 |
| Average | 6.78E+05 | 4.75E−03 | 5436 | 8.39E+04 | 2.00E−05 | 238 | |
| SD | 2.96E+05 | 4.00E−03 | 2210 | 3.42E+04 | 1.60E−05 | 220 | |
| Geometric mean | 6.45E+05 | 3.81E−03 | 5055 | 8.03E+04 | 1.65E−05 | 154 | |

All references cited herein, including patents, patent applications, papers, textbooks and the like, and the references cited therein, to the extent that they are not already, are hereby incorporated herein by reference in their entirety.

What is claimed is:

1. An antibody that binds to human TREM1, comprising:
a light chain variable region comprising:
a CDR-L1 comprising SEQ ID NO:11,
a CDR-L2 comprising SEQ ID NO: 12, and
a CDR-L3 comprising SEQ ID NO:13:
and a heavy chain variable region comprising:
a CDR-H1 comprising SEQ ID NO: 14,
a CDR-H2 comprising SEQ ID NO:15, and
a CDR-H3 comprising SEQ ID NO:16.

2. The antibody according to claim 1, wherein said antibody inhibits or attenuates TREM1 binding to one or more of its natural ligands.

3. The antibody according to claim 1 or claim 2, wherein said antibody inhibits or attenuates TREM1 binding to PGLYRP1.

4. The antibody according to any one of claims 1-3, wherein said antibody has a dissociation equilibrium constant (KD) of less than 600 μM for human TREM1.

5. The antibody according to any one of claims 1-4, wherein said antibody binds to a different site on TREM1 than PGLYRP1.

6. The antibody according to any one of claims 1-5, wherein said antibody binds to an epitope of human TREM1, the epitope comprising residues E26, E27, K28, Y29, E30, L31, K32 of human TREM1 (SEQ ID NO: 1).

7. The antibody according to any one of claims 1-5, wherein the antibody binds to an epitope of human TREM1, the epitope comprising five or more residues selected from E26, E27, K28, Y29, E30, L31, K32, Q35, T36, D38, K40, D42, R97, D127, T134 and G136 of human TREM1 (SEQ ID NO: 1) as determined at the distance of less than 4 Å contact distance between the antibody and TREM1.

8. The antibody according to claim 7, wherein said binding is determined using X-ray crystallography.

9. The antibody according to any one of claims 1-8, wherein the light chain variable region comprises the sequence given in SEQ ID NO:29.

10. The antibody according to any one of claims 1-9, wherein the heavy chain variable region comprises the sequence given in SEQ ID NO:79.

11. The antibody according to any one of claims 1-8, wherein the light chain variable region comprises the sequence given in SEQ ID NO: 29, or a sequence which is at least 90% identical thereto; and the heavy chain variable region comprises the sequence given in SEQ ID NO: 79, or a sequence which is at least 90% identical thereto.

12. The antibody according to claim 1, wherein each CDR either contains up to three amino acid substitutions, and wherein such amino-acid substitutions are conservative.

13. The antibody according to claim 1, wherein the remainder of the light chain and heavy chain variable regions have at least 90% identity to SEQ ID Nos: 29 and 79 respectively.

14. The antibody according to any one of claims 1-13, wherein said antibody is an antibody fragment.

15. The antibody according to claim 14, wherein said antibody fragment is Fab, Fab′, F(ab)₂, Fv, dsFv, scFv, or dsscFv.

16. The antibody according to any one of claims 1-13, wherein said antibody is a full length antibody.

17. The antibody according to claim 16, wherein said antibody is an IgG1, IgG1 LALA, IgG4, IgG4P, or IgG4P FALA.

18. The antibody according to claim 16, wherein the antibody is an IgG4P comprising a light chain comprising the sequence given in SEQ ID NO: 31 and a heavy chain comprising the sequence given in SEQ ID NO: 81.

19. The antibody according to any one of claims 1-8, wherein the antibody is an IgG4P and wherein the remainder of the of the light chain and heavy chain has at least 90% identity or similarity to SEQ ID NOs:31 and 81 respectively.

20. An antibody that cross-competes with the antibody of claim 1 for binding to a TREM1 epitope comprising residues E26, E27, K28, Y29, E30, L31, K32, and Q35 of human TREM1 (SEQ ID NO: 1.

21. An IgG4P antibody that binds to an epitope of human TREM1, the epitope comprising residues E26, E27, K28, Y29, E30, L31, K32, and Q35 of human TREM1 (SEQ ID NO: 1).

22. An isolated polynucleotide encoding the antibody according to any one of claims 1 to 21.

23. An expression vector carrying the polynucleotide of claim 22.

24. A host cell comprising the vector as defined in claim 23.

25. A method of producing the antibody of any one of claims 1 to 21, comprising culturing the host cell of claim 24 under conditions permitting production of the antibody, and recovering the antibody produced.

26. A pharmaceutical composition comprising the antibody of any one of claims 1 to 22, and a pharmaceutically acceptable adjuvant or carrier.

27. The antibody of any one of claims 1 to 22, or the pharmaceutical composition as defined in claim 29, for use in a method of treatment of the human or animal body by therapy.

28. The antibody of any one of claims 1 to 22, or the pharmaceutical composition as defined in claim 23, for use as a medicament.

29. Use of the antibody according to any one of claims 1-22 or the pharmaceutical composition according to claim 26 for the manufacture of a medicament.

30. The antibody as defined in any one of claims 1-22 or the pharmaceutical composition according to claim 26, for use in the treatment of a neurological disorder.

31. A method of treating or preventing a neurological disorder comprising administering a therapeutically effective amount of the antibody as defined in any one of claims 1-22, or a pharmaceutical composition as defined in claim 26, to a patient in need thereof.

32. Use of the antibody according to any one of claims 1-22 or the pharmaceutical composition according to claim 24 for the manufacture of a medicament for the treatment of a neurological disorder.

33. The antibody or pharmaceutical composition according to claim 30, the method of claim 31, or the use according to claim 32, wherein said neurological disorder is amyotrophic lateral sclerosis (ALS) or Alzheimer's disease.
